SEQUENCE LISTING (66 sequences)

SEQ ID NO: 1 - DNA, 20 bp, Artificial Sequence
  Collagen Type I forward primer
gccaagaagaaggccaagaa

SEQ ID NO: 2 - DNA, 18 bp, Artificial Sequence
  Collagen Type I reverse primer
tgaggccgtcctgtatgc

SEQ ID NO: 3 - DNA, 18 bp, Artificial Sequence
  Collagen Type I probe
acatcccagcagtcacct

SEQ ID NO: 4 - DNA, 23 bp, Artificial Sequence
  Collagen Type III forward primer
ctgcttcatcccactcttattct

SEQ ID NO: 5 - DNA, 20 bp, Artificial Sequence
  Collagen Type III reverse primer
atccgcataggactgaccaa

SEQ ID NO: 6 - DNA, 18 bp, Artificial Sequence
  Collagen Type III probe
aacaggaagttgctgaag

SEQ ID NO: 7 - DNA, 21 bp, Artificial Sequence
  COMP forward primer
gagatcgtgcaaacaatgaac

SEQ ID NO: 8 - DNA, 20 bp, Artificial Sequence
  COMP reverse primer
gaccgtattcacgtggaacg

SEQ ID NO: 9 - DNA, 16 bp, Artificial Sequence
  COMP probe
ctggctgtgggttaca

SEQ ID NO: 10 - DNA, 21 bp, Artificial Sequence
  Decorin forward primer
aagttgatgcagctagcctga

SEQ ID NO: 11 - DNA, 19 bp, Artificial Sequence
  Decorin reverse primer
ggccagagagccattgtca

SEQ ID NO: 12 - DNA, 18 bp, Artificial Sequence
  Decorin probe
atttggctaaattgggac

SEQ ID NO: 13 - DNA, 21 bp, Artificial Sequence
  18s RNA forward primer
gaggccctgtaattggaatga

SEQ ID NO: 14 - DNA, 20 bp, Artificial Sequence
  18s RNA reverse primer
cgctattggagctggaatta

SEQ ID NO: 15 - DNA, 17 bp, Artificial Sequence
  18s RNA probe
caagtctggtgccagca

SEQ ID NO: 16 - DNA, 18 bp, Artificial Sequence
  partial GAPDH forward primer
caagttccatggcacagt

SEQ ID NO: 17 - DNA, 18 bp, Artificial Sequence
  partial GAPDH reverse primer
ggcctttccgttgatgac

SEQ ID NO: 18 - DNA, 14 bp, Artificial Sequence
  GAPDH probe
ccgagcacgggaag

SEQ ID NO: 19 - DNA, 16 bp, Artificial Sequence
  SCX forward primer
cgcccagcccaaacag

SEQ ID NO: 20 - DNA, 19 bp, Artificial Sequence
  partial SCX reverse primer
ttgctcaactttctctggt

SEQ ID NO: 21 - DNA, 15 bp, Artificial Sequence
  SCX probe
tctgcaccttctgcc

SEQ ID NO: 22 - DNA, 20 bp, Artificial Sequence
  partial COL-I forward primer
gccaagaagaaggccaagaa

SEQ ID NO: 23 - DNA, 18 bp, Artificial Sequence
  COL-I reverse primer
tgaggccgtcctgtatgc

SEQ ID NO: 24 - DNA, 15 bp, Artificial Sequence
  partial COL-I probe
acatcccagcagtca

SEQ ID NO: 25 - DNA, 17 bp, Artificial Sequence
  partial COL-III forward primer
ctgcttcatcccactct

SEQ ID NO: 26 - DNA, 18 bp, Artificial Sequence
  partial COL-III reverse primer
atccgcataggactgacc

SEQ ID NO: 27 - DNA, 15 bp, Artificial Sequence
  partial COL-III probe
aacaggaagttgctg

SEQ ID NO: 28 - DNA, 18 bp, Artificial Sequence
  partial DCN forward primer
aagttgatgcagctagcc

SEQ ID NO: 29 - DNA, 18 bp, Artificial Sequence
  partial DCN reverse primer
ggccagagagccattgtc

SEQ ID NO: 30 - DNA, 15 bp, Artificial Sequence
  partial DCN probe
atttggctaaattgg

SEQ ID NO: 31 - DNA, 18 bp, Artificial Sequence
  partial BGN forward primer
tggacctgcagaacaatg

SEQ ID NO: 32 - DNA, 18 bp, Artificial Sequence
  partial BGN reverse primer
agagatgctggaggcctt

SEQ ID NO: 33 - DNA, 15 bp, Artificial Sequence
  partial BGN reverse primer
tctgagctccgaaag

SEQ ID NO: 34 - DNA, 22 bp, Artificial Sequence
  GAPDH forward primer
caagttccatggcacagtcaag

SEQ ID NO: 35 - DNA, 20 bp, Artificial Sequence
  GAPDH reverse primer
ggcctttccgttgatgacaa

SEQ ID NO: 36 - DNA, 14 bp, Artificial Sequence
  GAPDH probe
ccgagcacgggaag

SEQ ID NO: 37 - DNA, 16 bp, Artificial Sequence
  SCX forward primer
cgcccagcccaaacag

SEQ ID NO: 38 - DNA, 23 bp, Artificial Sequence
  SCX reverse primer
ttgctcaactttctctggttgct

SEQ ID NO: 39 - DNA, 15 bp, Artificial Sequence
  SCX probe
tctgcaccttctgcc

SEQ ID NO: 40 - DNA, 19 bp, Artificial Sequence
  TNMD forward primer
gccgcgtctgtgaaccttt

SEQ ID NO: 41 - DNA, 20 bp, Artificial Sequence
  TNMD reverse primer
cgccctccttggtagcagta

SEQ ID NO: 42 - DNA, 20 bp, Artificial Sequence
  TNMD probe
ctaggttactacccgtatcc

SEQ ID NO: 43 - DNA, 20 bp, Artificial Sequence
  COL-I forward primer
gccaagaagaaggccaagaa

SEQ ID NO: 44 - DNA, 18 bp, Artificial Sequence
  COL-I reverse primer
tgaggccgtcctgtatgc

SEQ ID NO: 45 - DNA, 18 bp, Artificial Sequence
  COL-I probe
acatcccagcagtcacct

SEQ ID NO: 46 - DNA, 20 bp, Artificial Sequence
  COL-III forward primer
ctgcttcatcccactcttat

SEQ ID NO: 47 - DNA, 19 bp, Artificial Sequence
  COL-III reverse primer
atccgcataggactgacca

SEQ ID NO: 48 - DNA, 19 bp, Artificial Sequence
  COL-III probe
aacaggaagttgctgaagg

SEQ ID NO: 49 - DNA, 20 bp, Artificial Sequence
  DCN forward primer
aagttgatgcagctagcctg

SEQ ID NO: 50 - DNA, 19 bp, Artificial Sequence
  DCN reverse primer
ggccagagagccattgtca

SEQ ID NO: 51 - DNA, 20 bp, Artificial Sequence
  DCN probe
atttggctaaattgggactg

SEQ ID NO: 52 - DNA, 22 bp, Artificial Sequence
  BGN forward primer
tggacctgcagaacaatgagat

SEQ ID NO: 53 - DNA, 20 bp, Artificial Sequence
  BGN reverse primer
agagatgctggaggcctttg

SEQ ID NO: 54 - DNA, 16 bp, Artificial Sequence
  BGN probe
tctgagctccgaaagg

SEQ ID NO: 55 - DNA, 17 bp, Artificial Sequence
  ELN forward primer
tggctgcggtccagttg

SEQ ID NO: 56 - DNA, 20 bp, Artificial Sequence
  ELN reverse primer
tgtgaccgaatccagcttga

SEQ ID NO: 57 - DNA, 17 bp, Artificial Sequence
  ELN reverse probe
tacccaagcatcgaaag

SEQ ID NO: 58 - DNA, 20 bp, Artificial Sequence
  COMP forward primer
gagatcgtgcaaacaatgaa

SEQ ID NO: 59 - DNA, 19 bp, Artificial Sequence
  COMP reverse primer
gaccgtattcacgtggaac

SEQ ID NO: 60 - DNA, 16 bp, Artificial Sequence
  COMP probe
ctggctgtgggttaca

SEQ ID NO: 61 - DNA, 18 bp, Artificial Sequence
  MHC-I forward primer
ggaagcgctcaggtggaa

SEQ ID NO: 62 - DNA, 19 bp, Artificial Sequence
  MHC-I reverse primer
ggcactgtcactgcttgca

SEQ ID NO: 63 - DNA, 15 bp, Artificial Sequence
  MHC-I probe
aggagggagctacgc

SEQ ID NO: 64 - DNA, 21 bp, Artificial Sequence
  MHC-II forward primer
aaccctcaccctgatcaccat

SEQ ID NO: 65 - DNA, 21 bp, Artificial Sequence
  MHC-II reverse primer
ggtggtcagccacaatgtctt

SEQ ID NO: 66 - DNA, 17 bp, Artificial Sequence
  MHC-II probe
tgagcacctgtggaggt

(19) United States
(12) Patent Application Publication
(10) Pub. No.: US 2017/0296700 A1
(43) Pub. Date: Oct. 19, 2017

(54) STEM CELL COMPOSITIONS, SYSTEMS AND USES THEREOF

(71) Applicant: Virginia Tech Intellectual Properties, Inc., Blacksburg, VA (US)

(72) Inventor: JENNIFER G. BARRETT, LEESBURG, VA (US)

(73) Assignee: Virginia Tech Intellectual Properties, Inc., Blacksburg, VA (US)

(21) Appl. No.: 15/511,871

(22) PCT Filed: Sep. 16, 2015
(86) PCT No.: PCT/US15/50493

§ 371 Date: Mar. 16, 2017

Related U.S. Application Data
(60) Provisional application No. 62/050,792, filed on Sep. 16, 2014.

(51) Int. Cl. A61L 27/38; C12N 5/0775; C12N 5/077; A61K 35/19; A61L 27/24; A61K 35/28; A61K 35/12

(52) U.S. Cl. CPC A61L 27/3834; A61K 35/28; C12N 5/0667; C12N 5/0663; C12N 5/066; A61K 35/19; A61L 27/24; A61K 2035/124; C12N 2533/90; A61L 2430/10

(57) ABSTRACT

Described herein are stem cells and stem cell compositions that can be used to treat soft tissue injuries, including tendon and ligament injuries. Also described herein are cellular scaffolds that can contain a stem cell or stem cell compositions described herein. Also described herein are soft tissue bioreactor devices. Also described herein are methods of using the stem cells, stem cell compositions, and soft tissue bioreactors and methods of treating tendon and ligament injuries.

### CROSS-REFERENCE TO RELATED APPLICATIONS

This application claims the benefit of U.S. Provisional Application Ser. No. 62/050,792, filed on Sep. 16, 2014, having the title “Methods For Stem Cell Therapy with Improved Efficacy” , the entirety of which is incorporated herein by reference.

### BACKGROUND

Soft tissue injuries, such as tendon and ligament injuries, are commonplace in all species including humans, dogs, and horses. Traditional therapies fail to provide adequate healing in a large percentage of cases, which result in chronic pain and loss of use or activity. As such, there exists a need for improved therapies for treatment of soft tissue injuries.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 shows an overhead view one embodiment of a soft tissue bioreactor.

FIG. 2 shows a close-up view of one embodiment of an actuator, an actuator base, and fasteners of the soft tissue bioreactor of FIG. 1.

FIG. 3 shows a close-up view of one embodiment of a load cell, linker, and load cell brace of the soft tissue bioreactor of FIG. 1.

FIG. 4 shows an embodiment of a soft tissue bioreactor system having multiple soft tissue bioreactors.

FIG. 5 shows an embodiment of a stacked soft tissue bioreactor system having multiple soft tissue bioreactors.

FIG. 6 shows one embodiment of a pair of soft tissue bioreactor clamps.

FIG. 7 shows one embodiment of a soft tissue in place between the pair of soft tissue bioreactor clamps of FIG. 6.

FIG. 8 shows a lateral view of one embodiment of a tendon clamp of FIG. 6.

FIG. 9 shows another lateral view of one embodiment of a tendon clamp of FIG. 6.

FIG. 10 shows a front view of one embodiment of a tendon clamp of FIG. 6.

FIG. 11 shows one embodiment of a culture vessel and an upper culture vessel brace of the soft tissue bioreactor of FIG. 1 removed from the soft tissue bioreactor.

FIG. 12 shows a close up view of one embodiment of the soft tissue bioreactor of FIG. 1 in use with a soft tissue graft between a pair of soft tissue clamps within a culture vessel.

FIG. 13 shows one embodiment of an electrospinning device configured to generate fibrous scaffolds.

FIG. 14 shows another view of the electrospinning device of FIG. 13.

FIG. 15 shows a cartoon of the anatomy of a canine shoulder.

FIG. 16 shows a magnetic resonance image demonstrating contrast-enhanced MRI of supraspinatus tendinopathy.

FIGS. 17A-17C show ultrasonographic images of normal supraspinatus tendon (FIG. 17A), injured (contralateral to the normal supraspinatus tendon) supraspinatus tendon

(FIG. 17B), and healing of the injured supraspinatus tendon four months post adipose stem cell/PRP treatment (FIG. 17C).

FIG. 18 shows a graph demonstrating the results from a gait analysis as measured by the total pressure index percent (TPI %) on a Gait Rite force mate before (Pre-Trx) and after (Post-Trx) treatment with adipose stem cells/ platelet rich plasma (ASC/PRP) injection of the supraspinatus tendon. Normal TPI % was 30 (P=0.014).

FIG. 19 shows a graph demonstrating supraspinatus tendinopathy cross sectional area before and after treatment with ASC/PRP (P =0.002).

FIG. 20 shows a graph demonstrating the relative cross sectional area (CSA) in control (rehabilitation only) and ACS/PRP treated groups upon initial exam and at least 12 weeks after treatment (P=0.042).

FIG. 21 shows a table demonstrating the measured platelet to white blood cell concentration ratios in a platelet rich plasma composition. Each box represents one formulation, with platelet concentration (X×10³ cells/microliter) on the left, and WBC concentration (X×10³ cells/microliter) on the right. The goal concentrations for each formulation in each box are depicted as a ratio in parentheses in each box, for example: 150/5 being 150×10³platelets:: 5×10³ WBC. The range of each is at the top of each column or to the left of each row.

FIG. 22 shows a graph demonstrating the concentration of platelets (Y-axis in X×10³ cells (or platelets) per microliter) in twenty different PRP formulations that represented each goal formulation.

FIG. 23 shows a graph demonstrating concentrations of white blood cells (WBCs) (Y-axis in X×10³ cells per microliter) in different PRP formulations that represented each goal formulation. FIG. 24 shows a graph demonstrating platelet derived growth factor (PDGF) levels in twenty different PRP compositions.

FIG. 25 shows a graph demonstrating transforming growth factor (TGF)-beta levels in twenty different PRP compositions.

FIG. 26 shows a graph demonstrating fibroblast growth factor-2 levels in PRP compositions (1A-4E) and whole blood (WB), platelets (PC), white cells (WC) and platelet poor plasma (PPP). The ratios of Platelet: WBC in the PRP compositions (1A-4E) correspond to those presented in FIGS. 22-23, with 1A corresponding to the formulation with a goal ratio of 1000:40 and going in order with 4E corresponding to 50/0.2 formulation.

FIG. 27 shows a graph demonstrating interleukin-1 (IL 1) beta levels in PRP compositions (1A-4E) and whole blood (WB), platelets (PC), white cells (WC) and platelet poor plasma (PPP). The ratios of Platelet:WBC in the PRP compositions (1A-4E) correspond to those presented in FIGS. 22-23, with 1A corresponding to the formulation with a goal ratio of 1000:40 and going in order with 4E corresponding to 50/0.2 formulation.

FIG. 28 shows a graph demonstrating interleukin-1 receptor antagonist (IL1 RA) protein levels in PRP compositions (1A-4E) and whole blood (WB), platelets (PC), white cells (WC) and platelet poor plasma (PPP). The ratios of Platelet:WBC in the PRP compositions (1A-4E) correspond to those presented in FIGS. 22-23, with 1A corresponding to the formulation with a goal ratio of 1000:40 and going in order with 4E corresponding to 50/0.2 formulation.

FIG. 29 shows a graph demonstrating stromal cell derived growth factor (SDF1) alpha in PRP compositions (1A-4E) and whole blood (WB), platelets (PC), white cells (WC) and platelet poor plasma (PPP). The ratios of Platelet:WBC in the PRP compositions (1A-4E) correspond to those presented in FIGS. 22-23, with 1A corresponding to the formulation with a goal ratio of 1000:40 and going in order with 4E corresponding to 50/0.2 formulation.

FIG. 30 shows a graph demonstrating the cell number of tendon progenitor cells (TPCs) and bone marrow mesenchymal stem cells (BMMSCs) following four days of culture on collagen groups. #, P≦0.05 for cell type between collage group. *, P≦0.05 between cell type within a collagen group.

FIG. 31 shows a graph demonstrating the cell number of tendon progenitor cells (TPCs) and bone marrow mesenchymal stem cells (BMMSCs) following seven days of culture on collagen groups. #, P≦0.05 for cell type between collage group. *, P≦0.05 between cell type within a collagen group.

FIG. 32 shows a graph demonstrating relative scleraxis (SCL) gene expression in bone marrow (BM) and tendon progenitor (TPCs) cells cultured on each collagen group (control (-) porcine (P), bovine (B), HP-bovine (A), and Rat tail (R). TPCs demonstrate significantly greater expression of SCL as compared to bone marrow MSCs (BM) cells.

FIG. 33 shows a graph demonstrating the results of a flow cytometry analysis for cell surface markers CD90, OCT4 and MHC II of TPCs plated on collagen plates. Values demonstrated are percentage of cells expressing a particular marker.

FIG. 34 shows a graph demonstrating relative gene expression of collagen I in TPCs and BM cells cultured on each collagen group (control (-), porcine (P), bovine (B), HP-bovine (A), and Rat tail (R)).

FIG. 35 shows a graph demonstrating relative gene expression of collagen III in TPCs and BM cells cultured on each collagen group (control (-), porcine (P), bovine (B), HP-bovine (A), and Rat tail (R)).

FIG. 36 shows a graph demonstrating relative gene expression of COMP in TPCs and

BM cells cultured on each collagen group (control (-), porcine (P), bovine (B), HP-bovine (A), and Rat tail (R)).

FIG. 37 shows a graph demonstrating relative gene expression of decorin in TPCs and BM cells cultured on each collagen group (control (-), porcine (P), bovine (B), HP-bovine (A), and Rat tail (R)).

FIG. 38 shows a graph demonstrating glycosaminoglycan (GAG) concentration relative to total DNA concentration in decellularized tendons seeded with either TPCs or BMMSCs.

FIG. 39 shows a table demonstrating the cell number and the geometric 95% confidence interval for collagen groups for TPCS and BMMSCs following 4 and 7 days of culture.

FIG. 40 shows a graph demonstrating collagen type I relative gene expression from BMMSCs and TPCs cultured on each collagen group (control, porcine HP-bovine, and rattus (rat tail), determined at 7 days of culture. * P≦0.05) between TPCs and BMMSCs within a collagen group.

FIG. 41 shows a graph demonstrating collagen type III relative gene expression from BMMSCs and TPCs cultured on each collagen group (control, porcine HP-bovine, and rattus (rat tail), determined at 7 days of culture. * P≦0.05) between TPCs and BMMSCs within a collagen group.

FIG. 42 shows a graph demonstrating COMP relative gene expression from BMMSCs and TPCs cultured on each collagen group (control, porcine HP-bovine, and rattus (rat tail), determined at 7 days of culture. * P≦0.05) between TPCs and BMMSCs within a collagen group.

FIG. 43 shows a graph demonstrating decorin relative gene expression from BMMSCs and TPCs cultured on each collagen group (control, porcine HP-bovine, and rattus (rat tail), determined at 7 days of culture. * P≦0.05) between TPCs and BMMSCs within a collagen group.

FIG. 44 shows a graph demonstrating IL 1-beta versus WBC counts in low platelet concentration [platelet] PRP. It was observed that when the PRP is 2× or lower concentrated for platelets, an increased WBC concentration was correlated with high IL 1-beta (R²=0.9704).

FIG. 45 shows a graph demonstrating the correlation between WBC concentration and IL-RA levels.

FIGS. 46A-46D show phase contrast photomicrographs demonstrating cell morphology of BMMSCs (FIGS. 46A-46B) and TPCs (FIGS. 46C and 46D) following about 5 days of culture on control (FIGS. 46A and 46C) and rat collagen type I (FIGS. 46B and 46D). Bar shown in FIG. 46D=100 μm.

FIG. 47 shows an embodiment of a tendon bioreactor that has an interchangeable, enclosed modular vessel containing an MSC-laden decellularized tendon graft with 10mm×35 mm of exposed surface area immediately following seeding.

FIG. 48 shows an embodiment of the uniaxial strain applied to a tendon in a tendon bioreactor. The duration of each construct spent in the bioreactor per day gradually increased from 0 to 30 to 60 minutes over the cultivation period.

FIGS. 49A-49E shows graphs demonstrating mRNA profiles of tenocytic marker genes scleraxis (SCX) (FIG. 49A), collagen types-I/III (COL-I (FIG. 49B) and COL-III (FIG. 49C)), decorin (DCN) (FIG. 49D), and biglycan (BGN) (FIG. 49E) varied by bioreactor protocol-3% strain induced a phenotype correlated with tenocytic differentiation and development. Data is reported by fold-change with respect to FDST. Data points that share a letter are not significantly different.

FIGS. 50A-50B show graphs demonstrating Construct ultimate tensile strength (FIG. 50A) and elastic modulus (FIG. 50B) were increased to native physiological levels by bioreactor culture at 3% strain. Data points that share a letter are not significantly different. Asterisks demarcate i-test significance from iDTS.

FIGS. 51A-51D show graphs demonstrating endpoint scaffold content of DNA (FIG. 51A), soluble collagen (FIG. 51B), and GAG (FIG. 51C) (as quantified by spectrophotometric assays) as well as cumulative GAG release into cell culture media was similarly assessed (FIG. 51D). Data points that share a letter are not significantly different as determined via one-way MANOVA. Asterisks demarcate i-test significance from iDTS.

FIG. 52 shows images of scaffolds that were successfully decellularized and reseeded at supraphysiological density relative to FDST. MSCs integrated into DTS and adopted a tenocytic phenotype, which did not change relative to strain amplitude.

FIG. 53 shows flow cytometry data (%) demonstrating cell surface markers present on stem cells derived from bone marrow (BM), adipose tissue (AD), and tendons (TN).

FIG. 54 shows an image of a tendon bioreactor in use.

FIG. 55 shows a graph demonstrating strain versus time of one embodiment of a protocol implemented in a bioreactor.

FIG. 56 shows a graphical representation of the experimental timeline of Example 8.

FIGS. 57A-57C show (FIG. 57A) representative image from a TN CFU assay: photograph converted to binary for automated counting; (FIG. 57B) results from CFU assay for BM, AD, and TN cells at P2, demonstrating the high proliferative capacity of TN cells; and (FIG. 57C) final DNA concentrations in bioreactor constructs suggested no differences in endpoint cellularity between groups.

FIG. 58 shows an image demonstrating Histological sections of DTS and bioreactor constructs stained with H&E, 5 μm thick sections. Cells acquired tenocytic morphologies and aligned along the axis of scaffold anisotropy. Bars=100 microns

FIG. 59 shows an image demonstrating Confocal microscopy of bioreactor constructs labelled with DAPI and calcein, approximately 100 μm-thick z-stacks. TN and BM MSCs integrated deeper into DTS than AD MSCs. Bars=200 microns.

FIG. 60 shows an image demonstrating Morphology and cell-cell connectivity in a representative single-plane fluorescence sample from the BM group. Bar=200 microns.

FIGS. 61A and B show Confocal top (FIG. 61A) and side (FIG. 61B views of a representative sample from the BM MSC group showing extensive recellularization of DTS.

FIGS. 62A-62J show relative gene expression data for SCX (FIG. 62A), TNMD (FIG. 62B), COL I (FIG. 62C), COL III (FIG. 62D), DCN (FIG. 62E), BGN (FIG. 62F), ELN (FIG. 62G), COMP (FIG. 62H), MHC-1 (FIG. 621), MHC-2 (FIG. 62J) in BM, AD, TN and FDST groups.

FIGS. 63A-63C show graphs demonstrating final (FIG. 63A) GAG and (FIG. 63B) soluble collagen content in bioreactor constructs did not reveal significant differences between cell types and (FIG. 63C) accumulation of GAG in media, calculated from aliquots obtained at each media change, suggests that attenuation of GAG loss from DTS was not cell type-dependent.

FIGS. 64A-64B show graphs demonstrating (FIG. 64A) elastic modulus and (FIG. 64B) failure stress of bioreactor constructs obtained by endpoint tensile tests. Failure stresses of cell-laden constructs were significantly greater following bioreactor culture. Constructs in the TN MSC group endured 6.1±1.7× greater stresses than matched DTS controls.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, cell biology, organic chemistry, biochemistry, botany, zoology, physiology, reproductive biology, veterinary or medical sciences, and the like, which are within the skill of the art. Such techniques are explained fully in the literature.

Definitions

As used herein, “about,” “approximately,” and the like, when used in connection with a numerical variable, generally refers to the value of the variable and to all values of the variable that are within the experimental error (e.g., within the 95% confidence interval for the mean) or within .+−.10% of the indicated value, whichever is greater.

As used herein, “cell,” “cell line,” and “cell culture” include progeny. It is also understood that all progeny may not be precisely identical in DNA content, due to deliberate or inadvertent mutations. Variant progeny that have the same function or biological property, as screened for in the originally transformed cell, are included.

As used herein, “adipocyte” refers to a cell type also known as a lipocyte or fat cell. Adipocytes are the cells that primarily compose adipose tissue, specialized in storing energy as fat.

As used herein, “chondrogenic cell” refers to a chondrocyte at any stage of maturation and may express one or more of the following markers: annexin VI, Col2a1(IIa), betal Integrin (CD29), N-cadherin (Ncad), N-cam (Ncam1), tenascin C (Tnc), sox9, CEP-68,

MMP13 (matrix metalloproteinase-13), Matrilin-1, Col9, 11-fibrau, Syndecan-3, Col2a1(IIb), and aggrecan.

As used herein, “chondrocyte” refers to a cell that produces one or more of the components of cartilage, including collagen and proteoglycans.

As used herein, “chondroblast” refers to an immature chondrocyte.

As used herein, “control” is an alternative subject or sample used in an experiment for comparison purpose and included to minimize or distinguish the effect of variables other than an independent variable.

As used herein, “positive control” refers to a “control” that is designed to produce the desired result, provided that all reagents are functioning properly and that the experiment is properly conducted.

As used herein, “negative control” refers to a “control” that is designed to produce no effect or result, provided that all reagents are functioning properly and that the experiment is properly conducted. Other terms that are interchangeable with “negative control” include “sham,” “placebo,” and “mock.”

As used herein, “mammal,” for the purposes of treatments, refers to any animal classified as a mammal, including human, domestic and farm animals, nonhuman primates, and zoo, sports, or pet animals, such as, but not limited to, dogs, horses, cats, and cows.

As used herein, “culturing” refers to maintaining cells under conditions in which they can proliferate and avoid senescence as a group of cells. “Culturing” can also include conditions in which the cells also or alternatively differentiate.

As used herein, “passage,” “passaging” and the like, in the context of cell culture refers to the process of subculturing a population of cells and includes physically removing a subset of cells from a cell population and expanding the subset separately from the original population in a fresh culture environment. As used herein “passaging” does not include simple media changes where no subset of the original population is isolated and propagated.

As used herein, “expansion” or “expanded” in the context of cells, refers to an increase in the number of a characteristic cell type, or cell types, from an initial population of cells, which may or may not be identical. The initial cells used for expansion need not be the same as the cells generated from expansion. For instance, the expanded cells may be produced by ex vivo or in vitro growth and differentiation of the initial population of cells. Expansion can also refer to allowing a cell population to undergo one or more cell division without passaging the cells.

As used herein, “differentially expressed,” refers to the differential production of RNA, including but not limited to mRNA, tRNA, miRNA, siRNA, snRNA, and piRNA transcribed from a gene or regulatory region of a genome or the protein product encoded by a gene as compared to the level of production of RNA by the same gene or regulator region in a normal or a control cell. In another context, “differentially expressed,” also refers to nucleotide sequences or proteins in a cell or tissue which have different temporal and/or spatial expression profiles as compared to a normal, reference, or control cell.

As used herein, “isolated” means separated from constituents, cellular and otherwise, in which the polynucleotide, peptide, polypeptide, protein, antibody, or fragments thereof, are normally associated with in nature. A non-naturally occurring polynucleotide, peptide, polypeptide, protein, antibody, or fragments thereof, do not require “isolation” to distinguish it from its naturally occurring counterpart.

As used herein, “concentrated” refers to a molecule, including but not limited to a polynucleotide, peptide, polypeptide, protein, antibody, or fragments thereof, that is distinguishable from its naturally occurring counterpart in that the concentration or number of molecules per volume is greater than that of its naturally occurring counterpart.

As used herein, “diluted” refers to a molecule, including but not limited to a polynucleotide, peptide, polypeptide, protein, antibody, or fragments thereof, that is distinguishable from its naturally occurring counterpart in that the concentration or number of molecules per volume is less than that of its naturally occurring counterpart.

As used herein, “separated” refers to the state of being physically divided from the original source or population such that the separated compound, agent, particle, or molecule can no longer be considered part of the original source or population.

As used herein, “differentiate” or “differentiation,” refers to the process by which precursor or progenitor cells (i.e., chondrogenic progenitor cells) differentiate into specific cell types, e.g., chondrogenic cells.

As used herein, “effective amount” is an amount sufficient to effect beneficial or desired biological, emotional, medical, or clinical response of a cell, tissue, system, animal, or human. An effective amount can be administered in one or more administrations, applications, or dosages. The term also includes, within its scope, amounts effective to enhance normal physiological function.

As used herein, “effective ratio of platelets to leukocytes (or white blood cells) refers to the ratio, not absolute amount, of platelets to leukocytes present in a platelet rich plasma preparation that can result in a decrease in the cross sectional area of a tendon lesion while minimizing an inflammatory response as evidenced by the expression level of one or more pro-inflammatory markers.

As used herein, “effective amount of stem cells” is an amount of stem cells sufficient to promote soft tissue lesion, such as a tendon lesion, regeneration over scar tissue formation when administered to a subject in need thereof

As used herein, “stem cell” refers to any self-renewing totipotent, pluripotent cell or multipotent cell or progenitor cell or precursor cell that is capable of differentiating into multiple cell types.

As used herein, “induced pluripotent stem cell” or “iPS cell” refers to a cell capable of differentiating into multiple cell types that is artificially derived (not naturally derived) from a non-pluripotent cell.

As used herein, “totipotent” refers cells that can differentiate and give rise to all cells types in an organism, plus the extraembryoinc, or placental, cells.

As used herein, “pluripotent” refers to cells that can differentiate and give rise to all of the cell types that make up an organism, except for the extraembryonic, or placental, cells.

As used herein, “multipotent” refers to cells that can develop into more than one cell type, but are more limited than pluripotent cells in the cell types that they can develop into.

As used interchangeably herein, “subject,” “individual,” or “patient” refers to a vertebrate organism.

As used herein, “mesenchymal stem cell” refers to multipotent cells that can differentiate into chondrocytes, osteocytes, and/or adipocytes, are adherent to plastic, and can express stem cell antigens such as CD31, CD34, CD40, CD49c, CD53, CD74, CD90, CD106, CD133, CD 144, cKit, Slams, or combinations thereof.

As used herein, “tendon progenitor stem cell” or “tendon progenitor cell,” refers to a cell that can be distinguished form a tenocyte by the presence of a stem cell marker, such as tenomodulin, Oct-4, SSEA-4 or combinations thereof, can differentiate into tenocytes, osteocytes, chondrocytes, and adipocytes.

As used herein, “substantially pure cell population” refers to a population of cells having a specified cell marker characteristic and differentiation potential that is about 50%, preferably about 75-80%, more preferably about 85-90%, and most preferably at least about 95% of the cells making up the total cell population. Thus, a “substantially pure cell population” refers to a population of cells that contain fewer than about 50%, preferably fewer than about 20-25%, more preferably fewer than about 10-15%, and most preferably fewer than about 5% of cells that do not display a specified marker characteristic and differentiation potential under designated assay conditions.

As used herein, “biocompatible” or “biocompatibility” refers to the ability of a material to be used by a patient without eliciting an adverse or otherwise inappropriate host response in the patient to the material or a derivative thereof, such as a metabolite, as compared to the host response in a normal or control patient.

As used herein, “biodegradable” refers to the ability of a material or compound to be decomposed by bacteria or other living organisms or organic processes.

As used herein, “therapeutic” refers to treating, healing, and/or ameliorating a disease, disorder, condition, or side effect, or to decreasing in the rate of advancement of a disease, disorder, condition, or side effect. The term also includes within its scope enhancing normal physiological function, pallative treatment, and partial remediation of a disease, disorder, condition or side effect.

The terms “treating” and “treatment” as used herein refer generally to obtaining a desired pharmacological and/or physiological effect. The effect may be prophylactic in terms of preventing or partially preventing a disease, symptom or condition thereof such as a soft tissue injury (e.g. tendon injury, tendinopathy, or ligament injury) The term “treatment” as used herein covers any treatment of a soft tissue injury in a mammal, particularly a human, and includes: (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions. The term “treatment” as used herein refers to both therapeutic treatment and prophylactic or preventative measures. Those in need of treatment include those already with the disorder as well as those in which the disorder is to be prevented.

As used herein, “preventative” refers to hindering or stopping a disease or condition before it occurs, even if undiagnosed, or while the disease or condition is still in the sub-clinical phase.

As used herein, “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

As used herein, “synergistic effect,” “synergism,” or “synergy” refers to an effect arising between two or more molecules, compounds, substances, factors, or compositions that is greater than or different from the sum of their individual effects.

As used herein, “additive effect” refers to an effect arising between two or more molecules, compounds, substances, factors, or compositions that is equal to or the same as the sum of their individual effects.

As used herein, “autologous” refers to being derived from the same subject that is the recipient.

As used herein, “allograft” refers to a graft that is derived from one member of a species and grafted in a genetically dissimilar member of the same species.

As used herein “xenograft” or “xenogeneic” refers to a substance or graft that is derived from one member of a species and grafted or used in a member of a different species.

As used herein, “autograft” refers to a graft that is derived from a subject and grafted into the same subject from which the graft was derived.

As used herein, “allogeneic” refers to involving, derived from, or being individuals of the same species that are sufficiently genetically different so as to interact with one another antigenically.

As used herein, “syngeneic” refers to subjects or donors that are genetically similar enough so as to be immunologically compatible to allow for transplantation, grafting, or implantation.

As used herein, “implant” or “graft,” as used interchangeably herein, refers to cells, tissues, or other compounds, including metals and plastics, that are inserted into the body of a subject.

As used herein, “immunogenic” or “immunogenicity” refers to the ability of a substance, compound, molecule, and the like (referred to as an “antigen”) to provoke an immune response in a subject.

As used herein, “exogenous” refers to a compound, substance, or molecule coming from outside a subject or donor, including their cells and tissues.

As used herein, “endogenous” refers to a compound, substance, or molecule originating from within a subject or donor, including their cells or tissues.

As used herein, “bioactive” refers to the ability or characteristic of a material, compound, molecule, or other particle that interacts with or causes an effect on any cell, tissue and/or other biological pathway in a subject.

As used herein, “bioactive factor” refers to a compound, molecule, or other particle that interacts with or causes an effect on any cell, tissue, and/or other biological pathway in a subject.

As used herein, “physiological solution” refers to a solution that is about isotonic with tissue fluids, blood, or cells.

As used herein, “donor” refers to a subject from which cells or tissues are derived.

As used herein, “extra cellular matrix” refers to the non-cellular component surrounding cells that provides support functions to the cell including structural, biochemical, and biophysical support, including but not limited to, providing nutrients, scaffolding for structural support, and sending or responding to biological cues for cellular processes such as growth, differentiation, and homeostasis.

Discussion

Soft tissue injuries, including tendon injuries, are a common occurrence in humans, horses, and dogs. These injuries, particularly tendon injuries, are difficult to treat and often result in progressive pain, lameness, injury, and loss of use. The etiology of tendinopathy is multi-factorial. In some cases, mechanical factors can contribute to tendon tears and once the tendon body is stretched beyond it elastic threshold, the tendon can fail. This can also be accompanied with inflammation of the tendon sheath and/or tendon degeneration. Tendon damage and degeneration can also occur when microtrauma forces are applied within the tendon's physiological threshold but the normal reparative mechanisms cannot keep up with the damage. Once damaged due to chronic stresses, the degenerative tendon is more prone to acute rupture than normal tendons during physiological loading and also have a reduced reparative potential. This acute on chronic presentation is very common in humans and tendinopathy accounts for about 30 to 50 percent of all sports related injuries and more than 48% of occupational maladies in humans.

Symptomatic tendinopathy is characterized by activity-related pain, focal tendon sensitivity and intratendinous structural changes. Affected tendons demonstrate significant structural changes including disordered, haphazard healing with an absence of inflammation and diffuse, fusiform, and/or nodular tendon thickening. Natural healing often occurs through the formation of scar tissue, which is less elastic and is structurally weaker than normal tendon, which can hinder or prevent the return of the human, dog, or horse to its previous level of activity. Indeed, many athletic careers are ended in response to an acute or chronic tendon injury.

Conventional treatments of tendon injuries include rest, controlled activity, rehabilitation, retraining, and various preventive techniques. Even in the best of programs, recurrence of the tendon injury is a common occurrence. More recently, regenerative medicine techniques have been employed to promote regenerative healing rather than repair via scar tissue formation. While various regenerative medicine techniques have been used to treat tendon injuries, the success of these treatments are varied and are far from having a standard protocol and are not without their limitations. As such there exists a long felt need for improved compositions, methods, devices and techniques that promote regenerative healing and can improve the success rate of recovery from tendon injuries.

With that said, described herein are stem cell compositions, platelet rich plasma compositions, conditioned serum compositions, methods of making the compositions, soft tissue bioreactors, and methods of treatment using the aforementioned compositions and devices that can result in regenerative healing of a soft tissue injury that can be more efficacious than current treatments. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Stem Cell Compositions, Plasma compositions, Conditioned Serum Compositions, and Methods of Making

Described herein are stem cell compositions, plasma compositions, and conditioned serum compositions. Also described herein are methods of making the aforementioned compositions.

Bone Marrow (MSCs)

Bone marrow MSCs, also referred to as bone marrow stromal cells, can differentiate into multiple cell lines, including bone, cartilage, fibrous connective tissue and tendons. Bone marrow MSCs can also secrete cytokines, growth factors, and other bioactive factors that can reduced inflammations, inhibit apoptosis within tissues, recruit circulating stems cells, and integrate and reform tissue. In some embodiments the bone marrow cells can be obtained from a suitable bone, (e.g. sternum, femur, and tuber coxae). The bone marrow aspirate can be centrifuged and the resulting cell pellet can be resuspended in a media containing low-glucose DMEM supplemented with 1% Penicillin/Streptomycin (Pen/Strep), glutamine, and 10% fetal calf serum (FCS). The resuspended cells can be plated on a cell/tissue culture plate in a MSC monolayer media (a media that can generate and maintain a MSC monolayer). After plating, the bone marrow cells can be fed every two days after they have attached (e.g. about 4 days). Cells can be used without passaging in a treatment or formulation as described elsewhere herein. In other embodiments, when cells are about 80% confluent, the cells can be passaged. Optionally, the cells can be assessed for homogenity and/or spindloid cell phenotypes. Plates that have cells with a spindle shape and demonstrate a homogenous monolayer of cells can be trypsinized. Optionally cells can be frozen in a cell freezing media. Frozen cells can be thawed and used in a treatment or formulation as described elsewhere herein.

The bone marrow MSCs can be autologous, allogeneic, xenogeneic, or syngeneic. The bone marrow MSCs can contain one or more bone marrow MSCs. The composition can contain about 1 to about 10×10¹⁰⁰ or more bone marrow MSCs. In some embodiments the composition can contain about 1 to about 50 million bone marrow MSCs. The cultured bone marrow MSCs can be subsequently used as described elsewhere herein.

Adipose Stem Cells

Adipose can be a rich source of stem cells. The compositions described herein can include adipose stem cells. Two main sources of adipose stem cells are described herein. The first source is adipose MSCs. The term “cultured adipose stem cells” as used herein refers to adipose stem cells that can be generated by isolation of adipose tissue from a donor and subsequent in vitro selective culturing to obtain the adipose MSCs or other type of adipose stem cell, such as preadipocytes. This term also includes adipose MSCs or other adipose stem cell that was prepared by selective culturing, which includes passaging of the cells, of the stromal vascular fraction (SVF). The second major source is stromal vascular fraction (SVF) adipose stem cells. The term “stromal vascular fraction adipose stem cells,” as used herein, refers to adipose stem cells that are derived after digesting adipose tissue with collagenase with minimal (no selective culturing) in vitro manipulation and do not undergo in vitro passaging. These two compositions of cells are discussed in greater detail below.

Adipose derived stem cells can have advantages over bone marrow MSCs. Adipose tissue can be relatively less invasive to harvest and can be more plentiful than bone marrow. Further, adipose tissue can have a greater concentration of stem cells as compared to bone marrow aspirate.

Cultured Adipose Stem Cells

In some embodiments, the composition can include cultured adipose stem cells. The cultured adipose stem cells can be generated from a harvested adipose tissue sample from a subject. The adipose can be obtained from any location on the subject. In some embodiments where the subject or donor is a human, the adipose can be obtained from the buttocks, back, thigh, arm, and/or abdominal region. In some embodiments where the subject or donor is a canine, the adipose can be obtained from the chest, the lateral tail head, and/or back. In some embodiments where the subject or donor is an equine, the adipose can be obtained from the lateral tail head and/or chest. In other embodiments, the adipose tissue sample can be a liposuction aspirate obtained from a subject.

In some embodiments, harvested adipose cells can be cultured in vitro using a suitable method, which includes cell expansion, cell passaging, and the addition of bioactive factors, to promote, maintain or select for stemness or induce differentiation down a mesodermal, ectodermal, or endodermal cell lineage. In some embodiments, the adipose tissue or liposuction aspirate can be digested with collagenase and separated into and adipose fraction and a infranatant fraction. The infranatant fraction can be inactivated and the stromal vascular fraction pellet can be obtained by centrifugation. The SVF pellet can be plated and then cultured in vitro, which can include one or more steps of cellular expansion, at least one passage of the cells, and stimulation of the cells by one or more bioactive factors to maintain or select for stemness or induce differentiation down a mesodermal (bone, fat, cartilage, muscle), ectodermal (endothelium, neurons, epidermis/skin), or endodermal (liver) cell lineage. In some embodiments, selective culturing of the adipose tissue can derive adipose MSC cells. The cultured adipose stem cells produced by this method can be positive for CD13, CD29, CD44, CD49d, CD90, CD105 or combinations thereof. The cultured adipose stem cells produced by this method can be negative for CD14, CD31, CD45, CD144 or combinations thereof.

The cultured adipose stem cells can be autologous, allogeneic, xenogeneic, or syngeneic. The cultured adipose stem cells can contain one or more adipose mesenchymal stem cells. The composition can contain about 1 to about 10×10¹⁰⁰ or more cultured adipose stem cells. In some embodiments the composition can contain about 1 to about 50 million cultured adipose stem cells. The cultured adipose stem cells can be subsequently used as described elsewhere herein.

Stromal Vascular Fraction (SVF) Adipose Stem Cells

In some embodiments, the composition can include a population of SVF adipose stem cells. The adipose to generate the SVF adipose stem cells can be obtained from any location on the subject. In some embodiments where the subject or donor is a human, the adipose can be obtained from the buttocks, back, thigh, arm, and/or abdominal region. In some embodiments where the subject or donor is a canine, the adipose can be obtained from the chest, the lateral tail head, and/or back. In some embodiments where the subject or donor is an equine, the adipose can be obtained from the lateral tail head and/or chest. In other embodiments, the adipose tissue sample can be a liposuction aspirate obtained from a subject. After obtaining the sample, an amount of the tissue sample can be minced and digested with collagenase. In some embodiments, the digested tissue sample can be filtered to remove connective tissue and other debris. The digested sample can be centrifuged to obtain a stromal vascular fraction pellet. The pellet can be washed one or more times. In some embodiments, the pellet can be washed in a phosphate buffered saline (PBS) solution. The PBS can be magnesium and calcium free. After washing the SVF pellet can be resuspended in an amount of adipose culture media. The amount of adipose tissue culture media can range from about 0.1 mL to about 100 mL. In some embodiments, the amount of adipose tissue culture media is about 10 mL. An amount of the resuspended pellet can be placed in a cell culture dish or flask. The resupended cells can be expanded without passaging to produce SVF adipose stem cells ready for use in a treatment. In some embodiments, the resuspended cells can be expanded between 1-7 cell divisions before using. In other embodiments, the resuspended cells can be expanded between 6-8 cell divisions before harvesting for use. In some embodiments, the total time from obtaining an adipose sample from a subject to the end of expansion can be 12-14 days or less.

The SVF adipose stem cell population can contain mixture of cell types, including MSCs, adipocytes, fibroblasts, smooth muscle cells, endothelial cells, blood cells, endothelial progenitor cells, preadipocytes, vasculature progenitor cells, hematopoietic progenitor cells, hematopoietic stem cells, pericytes, and supra-adventicial cells. In some embodiments, after expansion, but prior to use, the SVF adipose stem cell population can be sorted based on cell surface markers to obtain a SVF adipose stem cell population that is enriched for a particular type of cell. In some embodiments, the SVF adipose stem cell population can be sorted using fluorescence activated cell sorting (FACS). In some embodiments, the SVF adipose stem cell population can be sorted to obtain a population enriched for SVF adipose MSCs. This enriched population can then be used in a treatment described elsewhere herein. In some embodiments, the SVF adipose stem cell population or enriched population can contain about 1% to about 10% MSCs. In other embodiments, adipose stem cell population or enriched population can contain about 10% to 20%, 20% to 30%, 30% to 40%, 40% to 50%, 50% to 60%, 60% to 70%, 70% to 80%, 80% to 90%, or 90% to 100% SVF adipose MSCs.

The SVF adipose stem cells or enriched population of SVF adipose stem cells can be autologous, allogeneic, xenogeneic, or syngeneic. The SVF adipose stem cells or enriched population of SVF adipose stem cells can be subsequently used as described elsewhere herein.

Tendon Progenitor Cells

Also described herein are populations of tendon progenitor cells (TPCs). In some embodiments the tendon stem cells can express one or more cell bone marrow MSC cell surface markers. In some embodiments the TPCs or TPC composition can be made by isolating a piece of tendon tissue. In some embodiments the size of the piece can be about 2 cm×2 cm×6 cm. The tendon tissue can be dissected out from the outer covering of the tendon. The tendon tissue can be kept in a warm medial until further processed. The tissue can be optionally weighed. The tendon tissue can be placed in PBS supplemented with an antibiotic mixture. The tissue can be minced until fine pieces are generated. The minced tendon tissue can then be centrifuged at about 500× g for about 1-20 minutes. In some embodiments, the minced tendon tissue can be centrifuged at about 500× g for about 5 minutes to form pellet. The pellet can be washed twice by resuspending the removing the supernatant, resuspending the pellet in about 15 ml of PBS supplemented with antibiotic(s), centrifuging at 500× g for about 1-20 minutes, and repeating these steps one additional time.

After the final re-centrifuge, the final pellet can be resuspended in an amount of a digestion solution containing collagenase. The collagenase can be contained in serum-poor (e.g. 1% FBS) high-glucose DMEM supplemented with antibiotic(s) and L glutamine. The amount of collagenase can be 0.1% to 5% v/v in the digestion solution, In some embodiments, 10 mL digestion solution can be used can be about 10 mL to 1 g tendon solution. To digest the tendon tissue, the resuspended pellet can be incubated in the digestion solution for about 30 minutes to about 16 hours at about 25-40° C. In some emboiments, the resuspended pellet can be incubated in the digestion solution at about 37° C. In some embodiments, the resuspended pellet can be incubated in the digestion solution with shaking. The shaking can be about 100-200 rpm. In some embodiments, the shaking can be about 150 rpm.

After digestion, the resulting digest can be centrifuged at about 500×g for about 10 minutes. The resulting pellet can be resuspended in a media containing dispase and/or other protease. The media contain dispase can be a serum poor, high-glucose DMEM supplemented with an antibiotic. The resuspeded tendon cells can be incubated in the dispase containing media for about 45 minutes to about 1.5 hours. The incubation can take place at about 37° C. The incubation can take place with shaking at about 150 rpm.

After incubation in the cells in the media containing the protease can be centrifuged at about 500× g for about 1-20 min. In some embodiments the cells in the media containing the protease can be centrifuged at about 500× g for about 10 minutes. The resulting pellet can be resuspended in a tendon medium (e.g. high-glucose (4.5 g/dL) DMEM with glutamine; 1% Pen/Strep; 10% FBS; 10% Horse Serum).

The suspension can be filtered through a mesh filter (e.g. 100 micron mesh filter) by gravity filtration. Fresh media can be used to wash the filter to further collection of the cells. The collected cells can then be plated on cell culture plates or vessels. Cells can remain undisturbed until attachment (typically about 4 days). After attaching, the cells can be fed every 2 days. Cells can be harvested and used at any time, even if not passaged. Cells can be passaged when they are about 80% confluent. Plates and vessels that demonstrate spindle-shaped cells and a homogenous monolayer of cells can be trypsinized.

The TPCs obtained can be isolated at anytime after initial plating using cell culture techniques and resuspended in any of the other compositions described herein, such as PRP, and conditioned serum. The TPCs can be autologous, allogeneic, xenogeneic, or syngeneic. The compositions containing TPCs can contain about 1 to about 10×10¹⁰⁰ or more cultured TPCs. In some embodiments the composition can contain about 1 to about 50 million cultured TPCs. The TPCs can be subsequently used as described elsewhere herein.

Platelet Rich Plasma

Also described herein are platelet rich plasma (PRP) compositions. The PRP can contain a greater concentration or amount of platelets as compared to the plasma fraction of a whole blood sample obtained from a subject. The PRP can be autologous, allogeneic, xenogeneic, or syngeneic.

In some embodiments, the PRP composition can have can have a platelet derived growth factor (PDGF) level ranging from about 500 to 600 ng/mL about 6,000 to about 12,000 ng/mL of transforming growth factor-beta, about 95 to about 120 ng/ml fibroblast growth factor-2 (FGF-2), about 750 to about 1500 ng/ml interleukin 1-beta (IL-1beta), about 10 to about 30 ng/ml IL-1beta receptor (IL-1betaR) as measured by interleukin 1 receptor agonist, and/or about 1150 to about 1210 pg/ml of stromal cell derived growth factor 1-alpha (SDF-1alpha). In some embodiments, the concentration of platelets in the PRP can range from about 900×10³ platelets/μl to about 1200 platelets/μl. In some embodiments, the concentration of platelets in the PRP can be about 1000×10³ platelets/μl. In some embodiments, the leukocyte concentration in the PRP composition can range from about 0×10³ to about 10×10³ leukocytes/μl. In some embodiments, the concentration of leukocytes in the PRP composition is about 0.2×10³ leukocytes/μl.

In some embodiments, the platelet rich plasma compositions can contain an optimized ratio of platelets to leukocytes. In some embodiments, the ratio of platelets to WBCs is an effective ratio of platelets to leukocytes. In some embodiments, the PRP can have an effective ratio of platelet to leukocytes ranging from about 1000:0.2 to about 10000:10 (platelets×10³ to leukocytes×10³ per microliter). The PRP having an effective ratio of platelet to leukocytes can have a platelet derived growth factor (PDGF) level ranging from about 500 to 600 ng/ml, about 6,000 to about 12,000 ng/ml of transforming growth factor-beta, about 95 to about 120 ng/ml fibroblast growth factor-2 (FGF-2), about 750 to about 1500 ng/ml interleukin 1-beta (IL-1beta), about 10 to about 30 ng/ml IL-1beta receptor (IL-1betaR) as measured by interleukin 1 receptor agonist, and/or about 1150 to about 1210 pg/mi of stromal cell derived growth factor 1-alpha (SDF-1alpha).

The PRP can be made from whole blood (i.e. blood drawn directly from the body from which none of the components has been removed) obtained from a subject. The whole blood can be mixed with an anticoagulant. The whole blood can be centrifuged to obtain a plasma fraction and a pelleted platelet containing fraction. The whole blood can be centrifuged at about 200 to about 1500 g for about 5 to about 20 minutes. In some embodiments, the whole blood can be centrifuged at about 800 g for about 10 minutes. This can produce a plasma fraction containing platelets, a buffy coat layer, and a red blood cell layer. The supernatant containing the plasma fraction can be removed. The plasma fraction can be centrifuged at about 2,000 g to about 8000 g for about 5 to about 20 minutes. In some embodiments, the plasma fraction can be centrifuged at about 4000 g for about 10 minutes. This provides a platelet pellet and a platelet poor plasma (PPP) faction The plasma fraction obtained after this centrifugation can be referred to as a PPP fraction because it contains less platelets than the platelet pellet obtained.

All or a portion of the PPP can be removed. The platelets can be resuspended in a volume of the PPP that is smaller than the original volume of the PPP or other diluent. This forms the platelet rich plasma PRP composition. Optionally, an antibiotic, such as amikacin, gentamycin, kanamycin, neomycin, streptomycin, or tobramycin can be added to the PRP composition.

In some embodiments, the buffy coat layer (which contains WBCs) that is formed during the initial centrifugation, can be removed and saved. From the buffy coat, white blood cells can be added back into the final PRP to a desired ratio of platelets to WBC or a particular amount and/or concentration of WBCs.

The PRP can be subsequently used as described elsewhere herein.

Conditioned Serum

In some embodiments, it is desired to activate the platelets prior to use in a treatment or formulation and obtain a composition containing platelet produced bioactive factors. Such a composition is referred to herein as conditioned serum. Conditioned serum does not contain platelets or contains fewer platelets than platelet rich plasma because during the production of conditioned serum, the platelets are clotted and the clot is removed to obtain the final serum composition. Conditioned serum can contain platelet-produced bioactive factor(s). The conditioned serum can be autologous, allogeneic, xenogeneic, or syngeneic.

The conditioned serum can contain (Please describe any particular amounts, concentrations, or ratios of particular bioactive factors of interest that can be contained in the Conditioned serum). In some embodiments, the conditioned serum contains an optimized amount of leukocytes.

The conditioned serum can be made by exposing a PRP composition as described elsewhere herein to one or more clotting promoters and incubating the mixture until a clot has formed. Incubation can be conducted at about 25 to about 40° C. Incubation can occur for 30 minutes to 14 hours. Suitable clotting promoters include glass beads and calcium chloride.

After clotting, the clot can be removed from the serum or the serum can be separated from the clot to obtain the conditioned serum. The conditioned serum can be subsequently used as described elsewhere herein.

Formulations

The stem cell compositions and cell populations described herein can be contained in or provided to a subject, such as an active ingredient, in a formulation. Further, the PRP and conditioned serum compositions can be contained in or provided to a subject such as an active ingredient, in a formulation. As such, also described herein are formulations that can contain an amount, including a therapeutically effective amount, of a stem cell or other cell population or composition as described herein, and/or a PRP composition as described herein, and/or conditioned serum composition as described herein.

The formulations can be administered to a subject in need thereof. The subject in need thereof can be suffering from a soft tissue injury or disorder, such as a tendon or ligament injury or disorder. In some embodiments, the subject in need thereof can be suffering from tendinopathy.

Pharmaceutically Acceptable Carriers and Auxiliary Ingredients and Agents

The I formulations containing a amount of a stem cell composition or cell population, PRP composition, or conditioned serum composition as described herein can further include a pharmaceutically acceptable carrier. Suitable pharmaceutically acceptable carriers include, but are not limited to, water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition. In some embodiments, the pharmaceutically acceptable carrier includes a PRP composition as described elsewhere herein or a conditioned serum composition as described elsewhere herein. In some embodiments, a stem cell composition or population can be resuspended or diluted in a PRP composition as described elsewhere herein or a conditioned serum composition as described elsewhere herein.

The formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active composition

In addition to the amount of the stem cell compositions, PRP compositions, and/or conditioned serum compositions, the formulation can also include an effective amount of auxiliary active agents, including but not limited to, DNA, RNA, amino acids, peptides, polypeptides, antibodies, aptamers, ribozymes, guide sequences for ribozymes that inhibit translation or transcription of essential tumor proteins and genes, hormones, immunomodulators, antipyretics, anxiolytics, antipsychotics, analgesics, antispasmodics, anti-inflammatories, anti-histamines, anti-infectives, and chemotherapeutics. Such types of agents are generally known in the art.

Effective amounts of the Stem Cell Compositions, PRP compositions, and Conditioned Serum Compositions

The formulations described herein can contain an effective amount of a stem cell composition, PRP composition, conditioned serum composition or combinations thereof. In some embodiments, the stem cells can be diluted and/or resuspended within a PRP or conditioned serum composition. The effective amount can be based, inter alia, on the species of the subject, number of lesions being treated, size of lesions being treated, soft tissue being treated, severity of the injury, etc.

The effective amount of stem or other cells contained in the formulation can range from 1 cell per lesion to about 10×10¹⁰⁰ or more cells per lesion. The effective amount of

PRP can range from 0.1 ml to 20ml. In some embodiments, the effective amount of PRP can be about 0.1, about 0.5, about 1, about 2, or about 4 ml.

The effective amount of conditioned serum can range from 0.1 ml to 20 ml. In some embodiments, the effective amount of conditioned serum can be about 0.1, about 0.5, about 1, about 2, or about 4 ml.

In embodiments where there is an auxiliary active agent contained in the formulation in addition to the stem cell composition, PRP composition, or conditioned serum, or combination thereof, the effective amount of the auxiliary active agent can vary depending on the auxiliary active agent. In some embodiments, the effective amount of the auxiliary active agent ranges from 0.001 micrograms to about 1 milligrams. In other embodiments, the effective amount of the auxiliary active agent ranges from about 0.01 IU to about 1000 IU. In further embodiments, the effective amount of the auxiliary active agent ranges from 0.001 mL to about 1 mL. In yet other embodiments, the effective amount of the auxiliary active agent ranges from about 1% w/w to about 50% w/w of the total pharmaceutical formulation. In additional embodiments, the effective amount of the auxiliary active agent ranges from about 1% v/v to about 50% v/v of the total pharmaceutical formulation. In still other embodiments, the effective amount of the auxiliary active agent ranges from about 1% w/v to about 50% w/v of the total pharmaceutical formulation.

The auxiliary active agent can be included in the pharmaceutical formulation or can exist as a stand-alone compound or pharmaceutical formulation that is administered contemporaneously or sequentially with the stem cell composition, PRP, condition serum composition, or combination thereof. In embodiments where the auxiliary active agent is a stand-alone compound or pharmaceutical formulation, the effective amount of the auxiliary active agent can vary depending on the auxiliary active agent used. In some of these embodiments, the effective amount of the auxiliary active agent can range from 0.001 micrograms to about 1000 grams. In other embodiments, the effective amount of the auxiliary active agent can range from about 0.01 IU to about 1000 IU. In further embodiments, the effective amount of the auxiliary active agent can range from 0.001 mL to about 1 mL. In yet other embodiments, the effective amount of the auxiliary active agent can range from about 1% w/w to about 50% w/w of the total auxiliary active agent pharmaceutical formulation. In additional embodiments, the effective amount of the auxiliary active agent can range from about 1% v/v to about 50% v/v of the total pharmaceutical formulation. In still other embodiments, the effective amount of the auxiliary active agent can range from about 1% w/v to about 50% w/v of the total auxiliary agent pharmaceutical formulation.

Dosage Forms

In some embodiments, the formulations or auxiliary agents described herein can be provided in a dosage form. The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, intraurethral, parenteral, intracranial, subcutaneous, intramuscular, intravenous, intra-articular, intralesional, intratendinous, and intradermal. Such formulations may be prepared by any method known in the art.

Dosage forms adapted for intra-articular or intralesional administration can be discrete dosage units such as vials, syringes, or tubes. These can be supplied with or without needles or other administration apparatus. Other dosage forms will be appreciated by those of skill in the art.

Soft Tissue Bioreactors

Also described herein are soft tissue bioreactor that can be configured to apply a mechanical strain or pressure to a graft, such as a tendon or ligament graft. In some embodiments, can be seeded with one or more stem cell compositions and/or populations described herein. The soft tissue bioreactor described herein can be configured to utilize commercially available culture vessels, thus reducing cost.

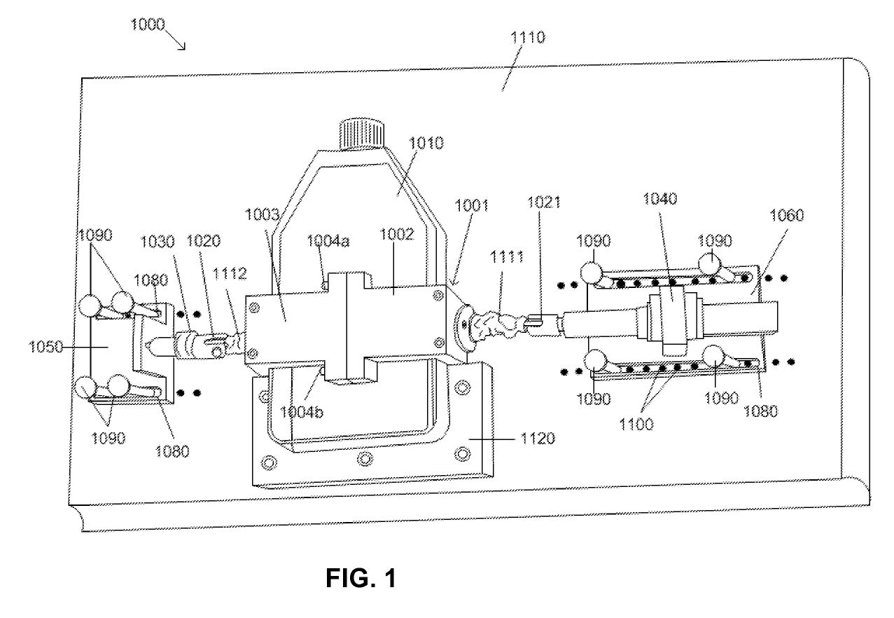

Discussion of the soft tissue bioreactor begins with FIG. 1, which shows an overhead view one embodiment of a soft tissue bioreactor 1000 containing an upper culture vessel brace 1001 configured to stabilize a culture vessel 1010 when in operation. The an upper culture vessel brace 1001 can contain two brackets 1002, 1003, that can be physically coupled to each other to form the upper culture vessel brace 1001 when coupled. The two brackets 1002, 1003 can be adjustably coupled to each other via one or more fasteners 1004a, b, such as a screw or other type of adjustable fastener that allows the brackets to be adjusted to allow a culture vessel 1010 to be placed into the soft tissue bioreactor 1000 as well as accommodate culture vessels 1010 of varying size and/or shape.

Although a tissue culture vessel 1010 having a rectangular body is shown in FIG. 1, any shape culture vessel 1010 can be used in the device. In some embodiments, the shape of the brackets 1002, 1003 are so dimensioned as to contour the shape of the culture vessel 1010 used in the soft tissue bioreactor 1000. This is shown for a rectangular shaped culture vessel 1010 in FIG. 1. The culture vessel 1010 can contain two or more holes (FIG. 12, 12000) through a surface, such as a side surface of the culture vessel 1010, that are in addition to a lid found on commercially available culture flask.

Both brackets 1002, 1003 contain an opening (FIG. 12, 12001) on one side of each bracket 1002, 1003 through which an arm of a soft tissue clamp can be passed through. The opening (FIG. 12, 120001) in each bracket can be aligned with one of the holes (FIG. 12, 12000) in the culture vessel 1010. These are shown more clearly in FIG. 7. The brackets 1002, 1003 can be made out of a polymer or co-polymer, metal or composite. The brackets 1002, 1003 can be coated with an antibacterial coating or microorganism controlling coating. In some embodiments, the brackets are made of Teflon® material.

As shown in FIG. 1, the arms of a first and second soft tissue clamp can be passed thorough each opening on the brackets 1002, 1003 and can be covered in a flexible sterile cover 1111, 1112 to maintain sterility of the graft that can be present inside the culture vessel 1010 during operation. The flexible sterile covers 1111, 1112 can be made of latex or other suitable material. The flexible sterile covers 1111, 1112 are oversized as compared to the size of the arms of the soft tissue clamp to allow the arms to slide during operation of the soft tissue bioreactor without tearing or excessive straining of the flexible sterile covers 1111, 1112.

The arm of the first soft tissue clamp can be releasably coupled to a first linker 1020 that can link the arm of the first soft tissue clamp to a load cell 1030. The arm of the second soft tissue clamp can be releasably coupled to a second linker 1021 that can link the arm of the second soft tissue clamp to an actuator 1040. The actuator 1040 can be configured to apply an axial strain to a graft held between the two soft tissue clamps when in use. In short, the actuator 1040 pulls on the tendon graft when in use to apply a mechanical stress to the graft. The actuator 1040 can be configured to provide a constant strain, an intermittent strain, a variable strength strain to the graft.

The load cell 1030 can be coupled to a load cell platform 1050, which can stabilize the load cell 1030 and can fixate the load cell 1030 in a single position. The actuator 1040 can be coupled to an actuator platform 1060, which can stabilize and/or fixate the actuator 1040 in one position to allow for operation. The load cell platform 1050 and the actuator platform 1060 can contain one or more slats 1080a, b, c, d through which fasteners 1090a-h can be passed. The fasteners 1090 a-h can be screws that can be screwed into holes (e.g. 1100a, b) in a soft tissue bioreactor platform 1110. The soft tissue bioreactor 1000 can also include a lower culture vessel brace 1120 that can assist in stabilizing and fixating the culture vessel 1010 during use. The lower culture vessel brace 1120 can contain one or more holes through which a fastener can be passed through. The fasteners can be passed through the hole(s) in the lower culture vessel brace 1120 and couple to the soft tissue bioreactor platform 1110. In some embodiments, the fasteners can be screws. Other fastener types will be appreciated by those of skill in the art. The use of screws or similar fasteners to secure the components of the soft tissue bioreactor 1000 to the soft tissue bioreactor platform 1110 allows the components to be adjustable to accommodate components of varying shapes and sizes. In some embodiments, the platform 1110 can be sized to fit within an incubator.

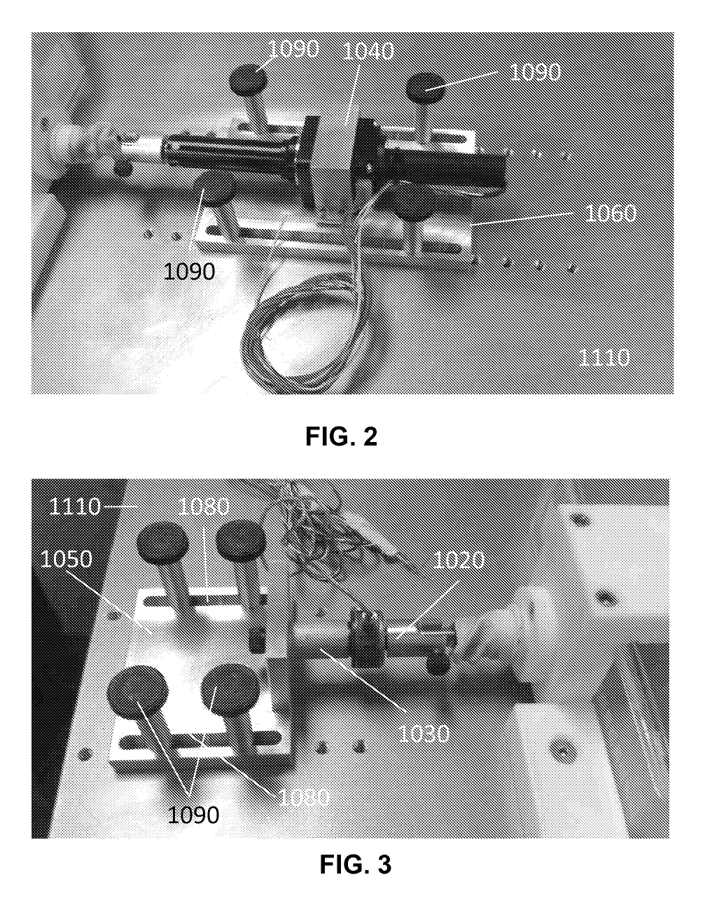

FIG. 2 shows a close-up view of the actuator 1040, the actuator base 1060, and fasteners 1090e-h, where the actuator 1040 is fixated on the soft tissue bioreactor platform 1110 via the fasteners 1090e-h passing through the slats 1080c,d in the actuator base 1060 and screwing into holes 1100a,b in the soft tissue bioreactor platform 1110. Power can be provided to the actuator by one or more wires electrically coupled to the actuator. Further, the actuator can be configured to receive a signal via a hardwire or wirelessly, where the signal controls the operation (on/off, strain strength, length of time, etc.) of the actuator. In other embodiments, the actuator can be in electrical or wireless communication with a controller configured to transmit a signal to the actuator. The controller can contain an operator interface, such as dials, keypad, buttons, toggles, a touch screen, and the like that allows an operator to control operation of the actuator.

FIG. 3 shows a close-up view of one embodiment of the load cell 1030, linker 1020 and load cell brace 1050. As shown in FIG. 3, the load cell brace can be coupled to the soft tissue bioreactor platform 1110 via fasteners 1090 (e.g. screws) that can be passed through slats 1080a,b in the load cell brace 1050.

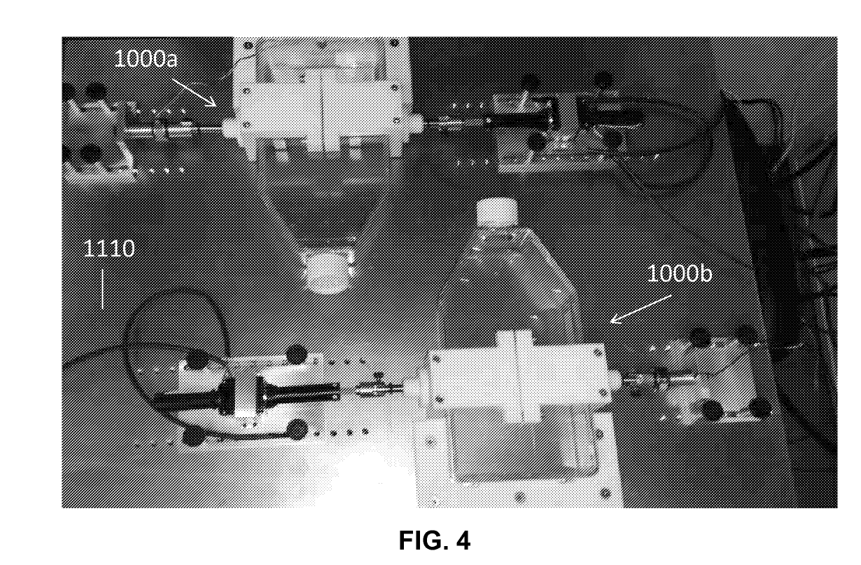

FIG. 4 shows an embodiment of a soft tissue bioreactor system 4000 where multiple (e.g. two) complete soft tissue bioreactors 1000a,b are coupled to the same soft tissue bioreactor platform 1110. From FIG. 4 it is easy to appreciate the scalability of the soft tissue bioreactor system described herein. As such, in other embodiments, any desired number of individual soft tissue bioreactors 1000a,b can be coupled to a single soft tissue bioreactor platform 1110. The size of the soft tissue bioreactor platform 1110 can be scaled accordingly to accommodate the desired number of soft tissue bioreactors 1000.

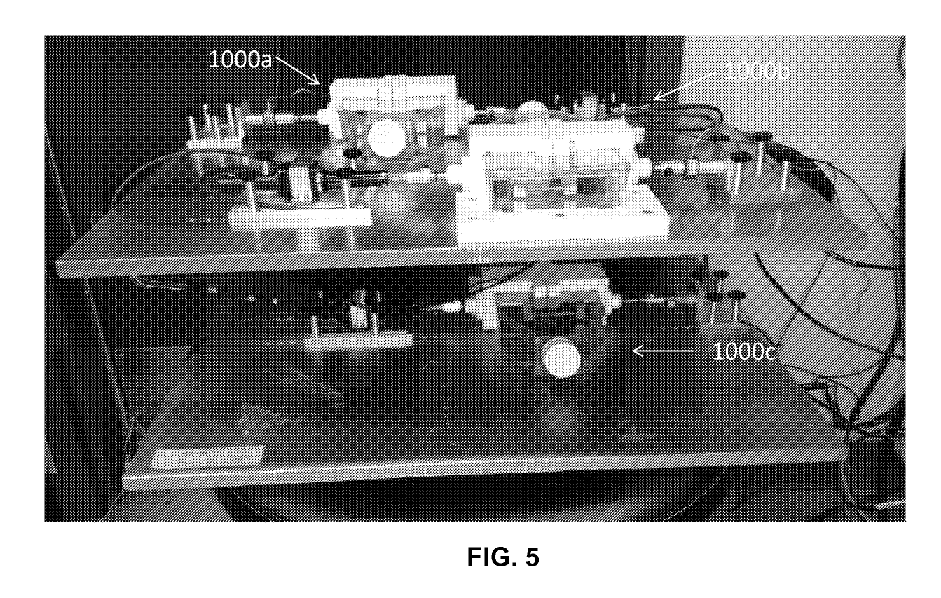

FIG. 5 shows an embodiment of a stacked soft tissue bioreactor system 5000, where multiple bioreactors 1000a-c can be contained on multiple soft tissue bioreactor platforms 1110a, b. This figure demonstrates the scalability of the soft tissue bioreactors described herein. In addition to be expanded in a horizontal direction, as shown in FIG. 4, the overall capacity of the system can be expanded vertically as well. The size of the individual platforms as well as the height of the stacks can be configured to fit within an incubator.

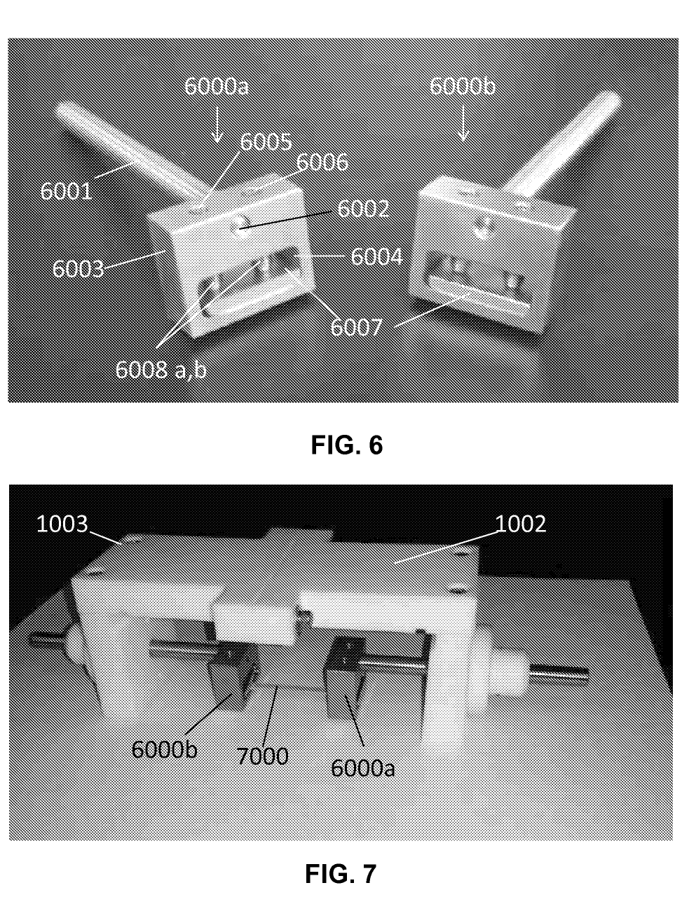

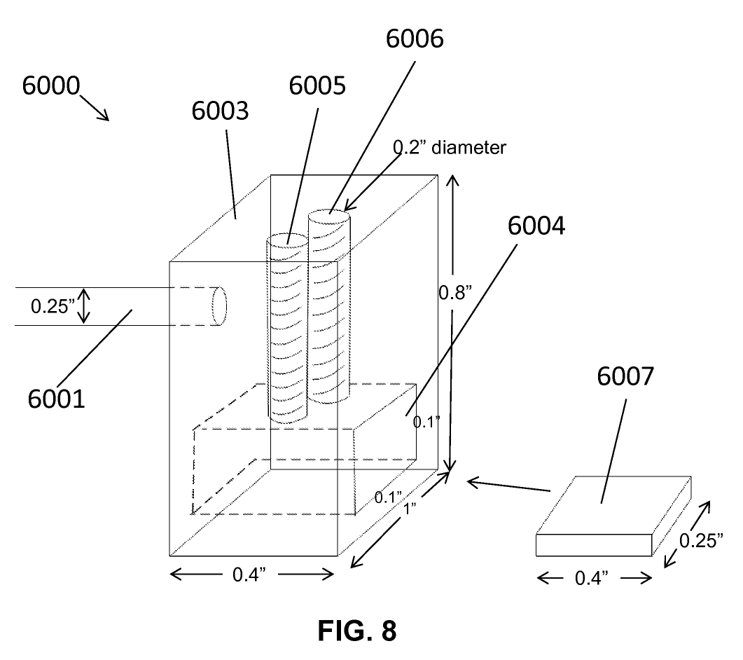

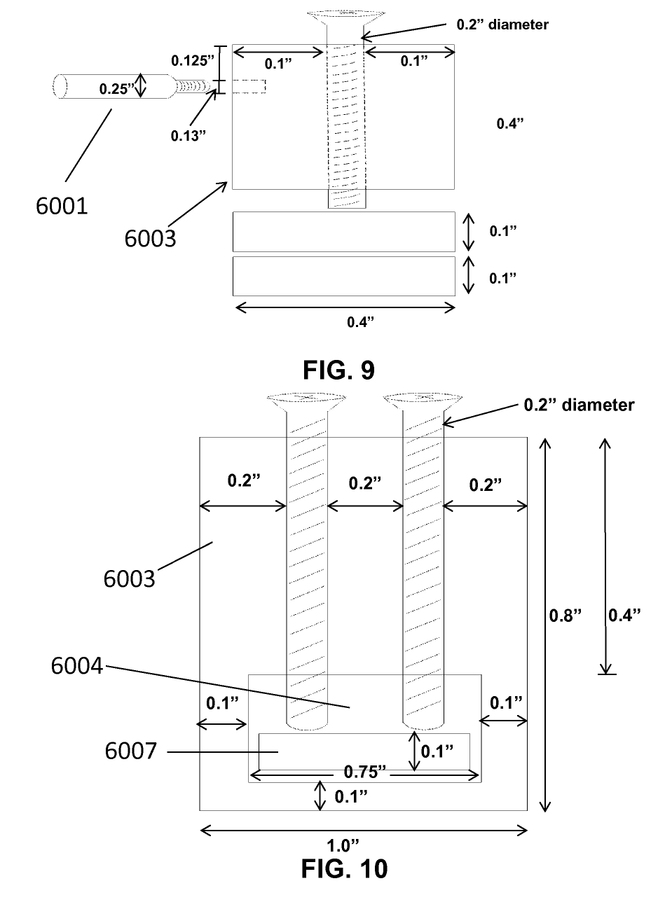

FIG. 6 shows one embodiment of a pair of soft tissue clamps 6000 a, b. As discussed with respect to FIG. 1 each soft tissue clamp 6000 can have an arm 6001 configured to pass through a hole in the side of the culture vessel 1010 and a hole in the side of the culture vessel bracket 1002 or 1003. Further, the arm 6001 can be configured to physically attach to a linker (FIG. 1, 1020 or 1021). The arm 6000 can have a thread at one end that can screw into a first hole 6002 with an opposing thread passing through a body portion 6003 of the soft tissue clamp 6000. The body portion 6003 can further contain a second hole 6004 that extends through the body portion 6003. The second hole 6004 can be larger than the first hole 6002 The body portion can further contain a third 6005 and a fourth hole 6006. The third hole 6005 and the fourth hole 60006 can extend through the top portion of the body 6003 and into the second hole 6004. Adjustable fasteners 6008 a,b, such as screws, can be passed through the third hole 6005 and the fourth hole 6006, such that one end of each adjustable fastener can pass through into the empty space in the body portion 6003 generated by the second hole 6004. The soft tissue clamp 6000 can further contain a soft tissue base 6007. The soft tissue base 6007 can be coupled to the one side of the second hole 6004, such that the adjustable fasteners 6008a,b can come in contact with the soft tissue base 6007 and not directly to the body portion 6003. FIGS. 8-10 shows several additional views of one embodiment of a soft tissue clamp demonstrating the configuration of the second, third, and fourth holes 6004, 6005, 6006, the adjustable fasteners 6008, and the soft tissue base 6007, and the arm 6001.

The soft tissue base can 6007 can be made out of the same material as the body portion 6003 or a different material. The soft tissue clamp, or any portion thereof, can be made out of a polymer, co-polymer, metal or metal composite. The soft tissue clamp or any portion thereof can be coated with an antimicrobial coating.

As shown in FIG. 7, in operation, the two soft tissue clamps are positioned in the soft tissue bioreactor 1000 (only the culture vessel brace is shown for clarity) such that they oppose one another. A soft tissue graft 7000, such as a tendon graft, can be held between the two soft tissue clamps 6000a,b. The soft tissue graft 7000 can be held between the two soft tissue clamps by placing one end of the soft tissue graft 7000 on top of the soft tissue base 6007 of one tendon clamp. 6000a and screwing down one or both the adjustable fasteners 6008a,b such that they pin the end of the soft tissue graft 7000 between the adjustable fastener(s) 6008 and the soft tissue base 6007. The other end (the free end) of the soft tissue graft 7000 can be secured in the other soft tissue clamp 6008b in a similar fashion.

While the description of FIG. 7 refers to a soft tissue graft, it will be understood that all types of soft tissue samples, whether a graft or not can be fitted within the soft tissue bioreactor 1000 in a similar manner. Further, the soft tissue bioreactor 1000 and systems can be used with any soft tissue sample or synthetic tissue scaffold. The soft tissue sample or other scaffold can be seeded with one or more of the stem cell or other cell compositions described herein. In some embodiments, the soft tissue graft can be a decellularized tendon graft seed with one or more of the stem cell or other cell compositions described herein. In operation the arms 6001a,b of the tendon clamps can be passed through holes 7001 in the culture vessel brackets 1002, 1003 and coupled to a load cell 1030 or an actuator 1040 to allow for an axial strain to be applied to the soft tissue graft 7000 held between the soft tissue clamps 6000a,b. A close up of a soft tissue graft 7000 held between the soft tissue clamps 6000a,b within the culture vessel 1010 is shown in FIG. 12.

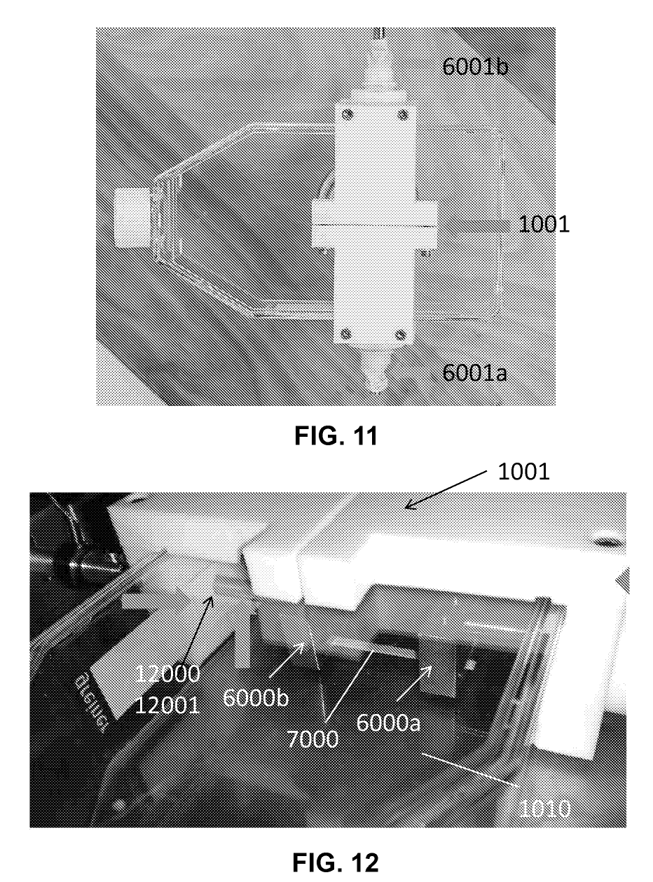

As shown in FIG. 11, the upper cell culture brace 1001 and the releasable arms 6001a,b of the soft tissue clamps 6000a, b can allow for easy removal of the cell culture vessel and the soft tissue graft within. This is advantageous when culture protocols demand periods of strain interposed with periods of rest. In other words, the soft tissue bioreactor can be configured to allow for easy removal of the culture flask to allow for periods of rest without tying up the actuator, which can be used on other samples during this time.

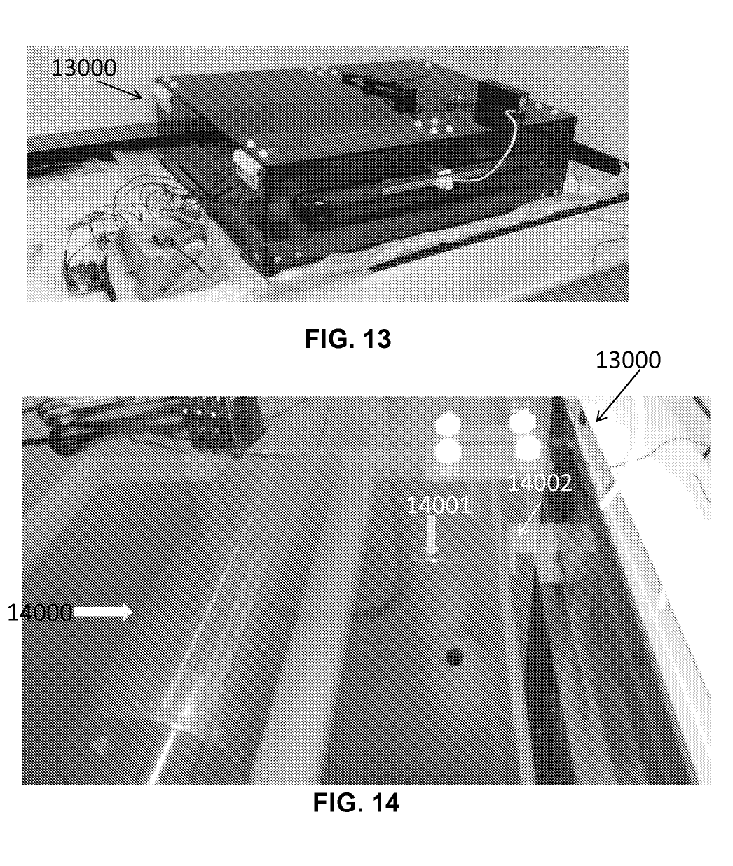

FIGS. 13-14 show several views of one embodiment of an electrospinning device 13000. The electrospinning device 13000 can be configured to manufacture fibrous scaffolds. The electrospinning device 13000 can contain a textured mandrel 14000 and a needle 14001. The electrospinning device can further contain a motor-driven belt, and a cassette 14002 coupled to the motor-driven belt. The cassette 14002 can be configured to hold the needle 14001. The cassette can be electrically coupled to a power source and be further configured to apply a voltage to the needle 14001 to charge the needle 14001. In operation, action of the motor-driven belt moves the cassette 14002 along the horizontal axis. Thus, in this way the needle 14001 can be moved along the horizontal axis. The textured mandrel can be coupled to an axel. The axel can be coupled to an adjustable motor. Action of the motor can rotate the axel and the textured mandrel 14000. The adjustable motor can include one or more sensors to detect rotations per second. The adjustable motor can be further configured to be responsive to a signal to control the rotation speed. The electrospinning device can also contain a syringe pump configured to pump a substrate, such as a scaffold polymer, through the charged needle 14001. In operation, polymers passing through the charged needle 14001 will produce fibers that can collect on a rotating mandrel 14000. The mandrel 14000, the cassette 14002 and needle 14001, the motor-driven belt, the adjustable motor, the axel, and syringe pump, can all be operatively coupled to or otherwise contained within an outer casing. In some embodiments, the outer casing can have multiple pieces and include a main body portion and a lid. In some embodiments, the power source can also be operatively coupled to or otherwise contained within the outer casing.

Methods of Treating Soft Tissue Injuries

Any cellular population or composition, including, the stem cell populations and compositions described herein can be suspended in a volume of any of the PRP or conditioned serum compositions described herein. The resulting compositions containing the stem cells described herein, a PRP composition and/or conditioned serum composition described herein can be administered by a suitable route, such as intra-artciular, intramuscular, subcutaneously, intravenous, and intralesional to a subject in need thereof. The subject in need thereof can have a soft tissue injury, such as tendinopathy. Administration can occur once or multiple times. When administration occurs multiple times, individual administrations can be spaced apart from one another with the time in between administrations ranging from 30 minutes to any number of months or years or more. In some embodiments, the time interval between administrations can be 1, 2, 3, 4, 5, 6, 7, 8, 9, 10, 11, or 12 weeks or more. In some embodiments where multiple administrations are given, the same composition can be administered each time. In some embodiments where multiple administrations are given, different compositions can be administered each time. In further embodiments where multiple administrations are given, the composition given at any one point in time is different that at least the previous composition administered.

In some embodiments, co-therapies are administered at the time of injection and in some cases, for a time interval following the injection or during the course of multiple injections. These co-therapies can include administration of an auxiliary agent as is described elsewhere herein, rest, rehabilitation, ice, compression therapy, shock therapy, magnet therapy, and vibration therapy. In some embodiments, the co-therapy can be administration of one or more auxiliary agents described elsewhere herein.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1

### Adipose Derived Stem Cells

Introduction Like humans, tendon injuries in dogs are difficult to treat and can result in chronic pain, lameness, and reinjury. Supraspinatus tendinopathy is a common condition found in active companion, working, and performance dogs across all breeds. Every year at the Veterinary Orthopedics and Sports Medicine Group (VOSM), 85-100 dogs are diagnosed with supraspinatus tendinopathy.

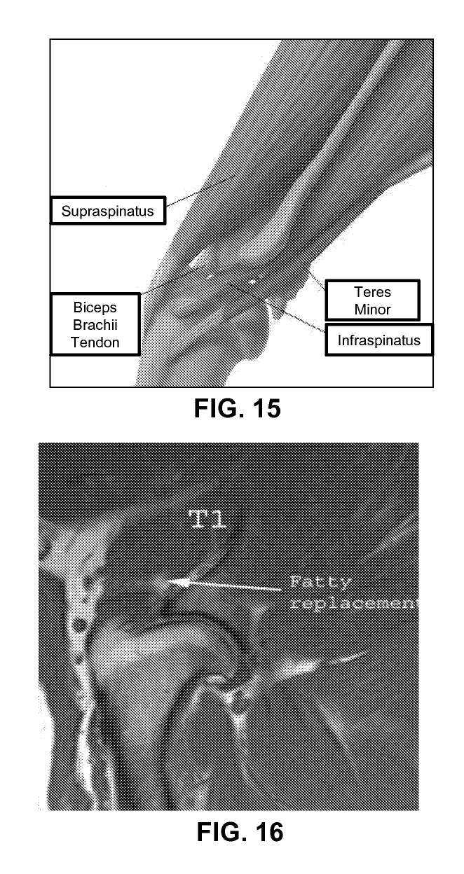

Supraspinatus Tendinopathy in Dogs: The shoulder joint is an intricate network of interlinked support mechanisms specially evolved to withstand large forces to provide extraordinary mobility while maintaining the stability and control necessary to enable precise function of the forelimb during activity. The supraspinatus muscle extends the shoulder and advances the limb (see FIG. 15). The muscle and tendon are critical to stabilize the shoulder joint and are active during weight bearing. Supraspinatus tendon injury can contribute to progression shoulder degenerative joint disease, and as such, early diagnosis and effective treatment are important. In both humans and dogs, several degenerative disorders of the supraspinatus tendon have been identified, including tears, tendonitis, or tendinosis (microtears), and generalized tendinopathy as a result of overuse. At the cellular level, affected tendons are observed to contain discontinuous and disorganized collagen fibers. In chronic cases, a rapidly growing nodule can develop that can impinge on the biceps brachii tendon and result in pain.

Performance dogs with supraspinatus tendinopathy typically present with weight bearing lameness on one side that becomes worse with activity and is resistant to conservative treatment. The supraspinatus muscle can become atrophied and direct palpation over the tendon and flexion of the shoulder cause pain. Shoulder instability can be present. The condition can be readily observed using magnetic resonance imaging (MRI) and/or ultrasound. FIG. 16 is a MRI image demonstrating contrast-enhanced MRI of supraspinatus tendinopathy. The white arrow indicates contract enhancement of the tendon in T1 sequence.

Traditional therapies for tendon injuries include controlled activity, rehabilitation, retraining, and various preventive techniques. While traditional treatments can assist in the healing time, traditional treatments rarely result in definitive disease resolution and recurrence of the tendon injury is common.

Regenerative medicine techniques have been applied with moderate success to tendon injuries. A common theme in these techniques includes administering stem cells to a subject with the aim at promoting regenerative healing of an injury as opposed to repair via scar formation. Adipose tissue can provide a rich source of stem cells that can be isolated and cultured in vitro. Adipose tissue can also be macerated and enzymatically digested to obtain a stromal vascular fraction containing adipose stem cells. Several systems for obtaining both cultured adipose stem cells and SVF adipose stem cells exist and can allow for collection, processing, and administration to occur on the same day.

While a number of commercially available cell-based therapies available for treatment of tendinopathy, these treatments are expensive ($1000 or more per treatment) and efficacy of these therapies varies widely. The varied success can depend on, inter alia, the source of the cells, processing of the cells, administration regime, and use (or absence of) additional co-therapies, such as platelet rich plasma.

It is demonstrated in this Example that SVF adipose stem cells are effective in treating degenerative tendinopathy and may be effective in treating inflammatory and other degenerative conditions, which may impact diseases of the liver kidney and/or nervous system.

Methods

A retrospective study describing supraspinatus tendinopathy in 185 canine patients who presented to VOSM was conducted.

Signalment: The age of the population ranged from eight months to 14 years (average 6.3 y; median 6 years). No sex predisposition was apparent with 79 female dogs (10 intact) and 106 male dogs (19 intact). Breed representation included a majority of Labrador Retrievers, Border Collies, Golden Retrievers, and German Shepard Dogs. Performance/sporting dogs accounted for about one third (67) of the population. Companion animals accounted for the remaining two thirds (118). Of the performance/sporting dog population, 48% participated in agility.

History and Previous Treatment: At presentation, 73% of the dogs had a history of failed response (continued or recurrent discomfort/lameness) to rest and non-steroidal anti-inflammatory drug (NSAID) therapy (134). The seventy five failed to respond to rehabilitation therapy and 6 received intra-articular injections of hyaluronic acid-2; methylprednisolone acetate-4, which also did not relieve lameness.

Clinical Findings: Unilateral forelimb lameness presentation was more common than bilateral; =124 and 61 respectively. Lameness was graded from 0 (sound) to 6 (non-weight bearing lame) on a subjective scale. The average lameness score was 2.9 (median 3). Duration of lameness ranged from one week to more than one year. 29.7% of dogs had a chronic (>1 year) lameness. Of those with lameness less than one year, the average duration was 15.2 weeks (median 6 weeks).

On physical examination, pain on direct palpation of the supraspinatus was found in 59% of dogs. Pain/spasm on flexion was recorded in 74.6% of dogs and pain on biceps stretch (shoulder flexion with elbow extension) was recorded in 47.5% of dogs.

Diagnostic imagining: Shoulder radiographs were performed on 161 dogs. Of these, mineralization was noted in 35 supraspinatus tendons. Thirty-tree percent of dogs were diagnosed with bilateral disease on presentation. Radiographs of the ipsilateral elbow were performed in 145 dogs. Of these, abnormalities (sclerosis, osteoarthritis, fragments, etc.) were noted in 85 dogs. Overall over 50% of dogs who presented had unilateral supraspinatus tendon injury without concurrent elbow or shoulder injuries.

MRI of the shoulder was performed in 30 cases. Findings indicative of a supraspinatus tendinopathy on MRI in all 30 cases included some or all of the following findings: hyperintensity of signal on T1 and STIR sequences of the supraspinatus tendon at its insertion on the greater tubercle mineralization, flattened or oval appearance of the biceps tendon, loss of fluid around the biceps tendon within the bicipital groove a the level of insertion of the supraspinatus and subsequent compartmentalization of fluid distal to supraspinatus insertion, fatty replacement at myotendinous junction of the supraspinatus and/or displacement of the biceps tendon from the bicipital groove characterize biceps impingement secondary to supraspinatus tendinopathy.

Diagnostic ultrasonography was performed in 140 cases. Common findings indicative of supraspinatus tendinopathy included increased size, irregularities in shape, mineralization and reduction in echogenicity.

Surgical Findings: Shoulder arthroscopy was performed in 131 cases. Of these, elbow arthroscopy was performed in 115 cases and 7 dogs had elbow arthroscopy only. On shoulder arthroscopy, a supraspinatus bulge was noted in 90.8% of dogs. Of these, impingement of the biceps tendon was noted in 59.9% of dogs. While 43.5% of dogs had bicipital tenosynovitis, only 13% had actual pathology of the biceps tendon. Of the 115 dogs that had elbow arthroscopy, 64.3% had pathology of the elbow (FCP/MCD). Overall, over 50% of dogs had unilateral supraspinatus tendon injury with no elbow pathology and no shoulder degenerative joint disease.

Treatments: The treatment plan for cases that do not respond to rehabilitation and conservative management included ultrasound-guided intratendionous injections of adipose stem cells/plasma rich platelet (ASC/PRP) compositions. ACS/PRP compositions were prepared as described by the following methods. Adipose and blood samples were obtained aseptically and, where not processed immediately, were stored at 4° C. For processing, at least the following materials were used:

5.1 Clean cuffed laboratory coat

5.2 Hair cover

5.3 Gloves

5.4 Sample File and pen

5.5 Adipose culture media

5.6 Digest Media (media suitable for collagenase digestion)

5.7 250 ml filtration unit (Nalgene)

5.8 Collagenase Type 2 (Worthington Biochemical); 0.175 g premeasured into 50 ml conical tube

5.9 Sterile fitted gloves

5.10 Sterile scissors and forceps

5.11 Sterile nylon cell strainer (100 μm); BD Falcon

5.12 Phosphate buffered saline (PBS) without calcium or magnesium

5.13 Disposable sterile serological pipettes (individually wrapped)

5.14 PipetAid serological pipettor

5.15 Pasteur pipettes, sterilized and endotoxin free

5.16 Kimwipes®

5.17 T-175 culture flasks

5.18 70% ethanol (spray bottle)

5.19 Distilled water—Millipore (spray bottle)

5.20 Vacuum pump

5.21 Vacuum traps (plastic flasks and associated tubing)

5.2.2 Sterile 50/15 ml screw cap conical centrifuge tubes

5.23 Plastic test tube racks

5.24 Permanent marking pen

5.25 Lab timers

5.26 37° C. bead bath

5.27 Ohaus scale (Adventure-Pro)

5.28 Air Circulating Incubator Shaker Unit

5.29 Refrigerated bench top centrifuge

5.30 Heracell CO2 Incubators

5.31 Biological safety cabinet

ASC (Adipose Stem Cell) Preparation

Autologous SVF adipose stem cells were prepared using the following procedure. After putting on the cleaned cuffed laboratory coat, Tyvek® sleeves, hair cover, and clean gloves, the biosafety cabinet was prepared and appropriate media and reagents were placed in the 37° C. bead bath to warm. This should be performed prior to receiving the sample. If processing cannot begin immediately, the sample can be stored at 2-8° C. until processing can begin. Samples should be processed within 12 hours of receiving them. The air circulating incubator shaker unit was turned on and warmed. The appropriate reagents and supplies for sample processing were placed in the biosafety cabinet and cleaned with 70% ethanol. While observing aseptic techniques, the collagenase was completely dissolved in 50 mL of digest media. The dissolved collagenase was filtered through the 250 mL vacuum filter.

About 8-10 grams of adipose tissue was cut into small (about 0.5 cm) pieces and digested in the filtered collagenase containing digest media. To digest the adipose tissue, the pieces of adipose tissue in filtered collagenase containing digest media was incubated at about 37° C. with shacking (about 150 rpm) in the air circulating incubator shaker. Digestion was allowed to continue until the solution did not contain any pieces of tissue greater than about 3 mm³, typically about 30 minutes. While incubating, the corresponding blood sample was processed for PRP (discussed below). The nylon cell strainers were each tilted on top of three conical centrifuge tubes. Using a serologic pipette, the cell suspension was slowly and carefully transferred into the cell strainers over the 50 mL conical tubes. After filtering the suspension and removing the cell strainers, the filtered suspension was divided equally among the 50 mL conical tubes.

The filtered suspension was centrifuged at about 800 g for 10 minutes with refrigeration (Centrifuge setting: Sorval Mach 1.6- up=9, down=3; Sorval X1R- up=9, down=9). After centrifugation, the supernatant was aspirated, being careful to avoid disturbing the pellet that formed at the bottom of each tube. After aspirating the supernatant, about 10 mL of PBS (minus calcium and magnesium) was added to each tube and the pellet was resuspended in the PBS via trituration taking care not to aspirate the suspension too high in the pipette. After resuspension, the cell suspensions were combined into 2 tubes and the volume in each tube was up to 40 mL per centrifuge using PBS where necessary.

The combined cell suspensions were centrifuged at 800 g for 10 minutes under refrigeration. After this centrifugation, the steps of aspiration, resuspension in 10 mL PBS, bringing to a final volume of 40 mL in PBS, and centrifugation at 800 g for 10 minutes under refrigeration was repeated once. After the centrifugation step, the cells pellet was resuspended in PBS to a final volume of 10 mL in each tube for a total volume of about 20 ml. The two cell suspensions (totaling 20 mL) were combined and an aliquot was removed and used to quantify the nucleated fraction. After the aliquot was removed the cell suspension was centrifuged again at 800 g for 10 minutes under refrigeration (2-8° C.) After this final centrifugation, the SVF adipose stem cells were finally resuspended in about 10 mL of APC media.

5 mL of the resulting cell suspension was added to each T-175 cell culture flask containing about 45 mL pre-warmed APC media. The cells were cultured through about 6-8 cell divisions without passaging. Typical time from tissue removal to having SVF adipose stem cells ready for administration is about 7-14 days.

PRP Preparation

Whole blood samples were obtained and maintained at about 2-8° C. if not processed immediately. While practicing aseptic technique a biosafety cabinet and reagents were prepared. Where necessary, media and other reagents were pre-warmed to about 37° C. The sample should be processed within about 12 hours of receipt.

To begin, the whole blood samples were gently mixed by inversion or rolling between palms and transferred to 50 mL conical tubes while being careful to avoid overfilling of the conical tubes. A small aliquot was kept for whole blood evaluation. The whole blood samples were then centrifuged at about 800 g for about 10 minutes under refrigeration. Centrifuge settings were: Sorval Mach 1.6- up=9, down=1; Sorval X1R- up=9, down=6. The final amount of PRP needed was calculated (typically about 2 mL per lesion per injection in canines).

After the initial centrifugation, the supernatant was transferred to a new tube. The size of the new tube depended on the amount needed previously calculated. The supernatant was carefully removed such that the buffy coat resting on top of the red blood cell pellet was undisturbed. To avoid the buffy coat a small amount of the supernatant can be left in the tube. The supernatant, which contains the plasma fraction, should be cloudy as the platelets are suspended throughout the plasma. After removal, the supernatant was centrifuged for about 10 minutes at 4000 rpm. Centrifuge settings were: Sorval Mach 1.6-up=9, down=3; Sorval X1R- up=9, down=9. After centrifugation, the platelets form a pellet and the remaining plasma was the platelet poor plasma (PPP). Depending on the volume required for treatments, some of the PPP can be removed and saved. The platelet pellet was resuspened in a volume of PPP based on the total amount of PRP needed that was previously calculated. The volume of PPP that the platelet pellet was resuspended in was smaller than the initial PPP fraction obtained after centrifugation. When resuspending the platelet pellet, care was taken to avoid aspirating the pellet or creating bubbles that can trap the platelets. Instead, resuspension took place by slowly “washing” the pellet with the PPP until the pellet was completely suspended in the PPP.

In instances where the PRP was to be shipped or administered on the same day as processing, the following protocol was performed. The total amount of PRP needed for treatment was determined by multiplying the number of lesions by 2 ml. 25 μl of amikacin was added to the PRP for every 2 mL of PRP. This was accomplished by adding 25 μl of amikacin for every 2 mL PRP to an empty 50 mL conical tube. Then, the needed amount of PRP was added to the aliquot of amikacin and the combination was mixed gently without introducing air bubbles or aspirating the sample.

In all cases, a sample of the final PRP (with or without amikacin) was obtained for clinical lab testing. PPP and PRP that is not shipped or administered was frozen at −80° C.

ASC/PRP Preparation and Administration

To prepare the ASC/PRP, non-passaged but divided ASCs were removed from the cell culture flask and resuspended in PRP. For each injection, about 2 mL of PRP containing about ASCs were used. An intratendinous injection of the prepared ASC/PRP composition was administered using ultrasound to guide the injections to the lesions.

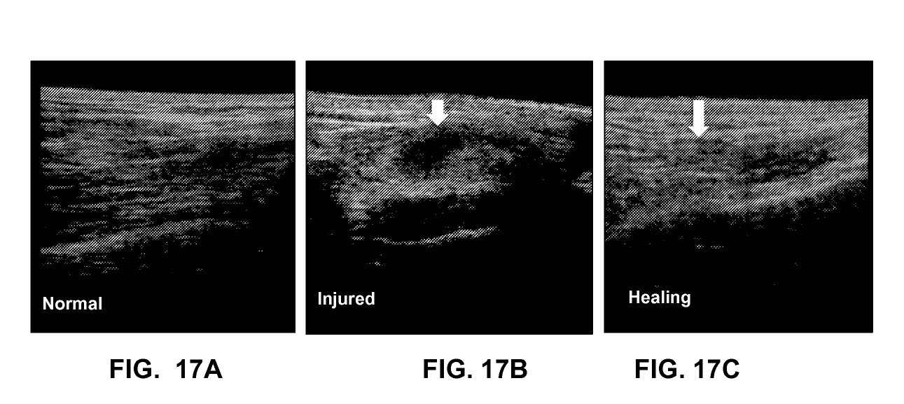

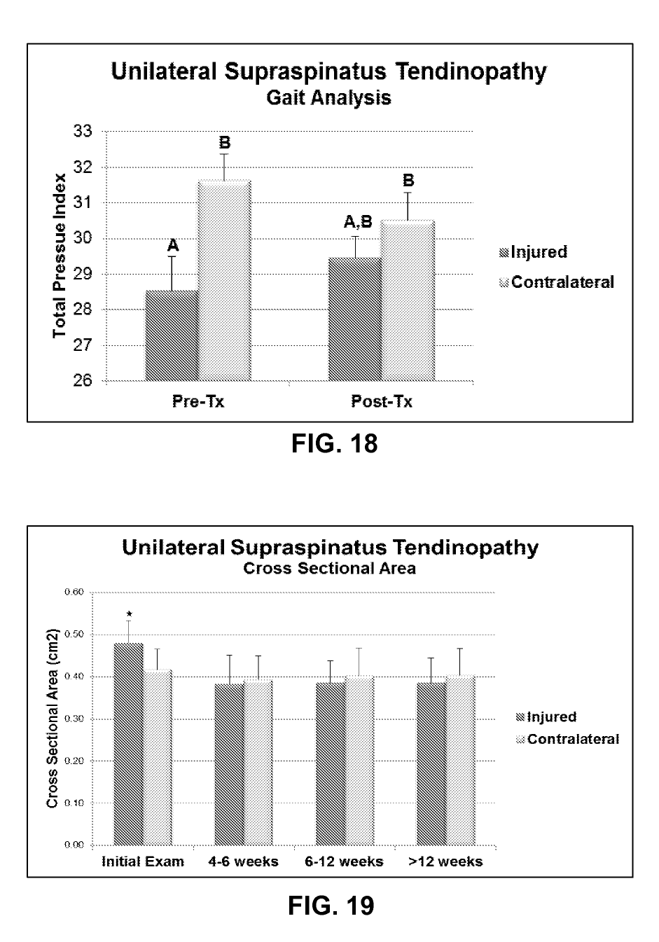

Outcomes after ASC/PRP therapy Follow up evaluations were performed for 92 dogs at least 90 days after ASC/PRP therapy of supraspinatus tendinopathy. Lameness resolved in 90% of cases, as evidenced by a return to normal total pressure index on objective gait assessment. Further an improvement was noted in the remaining 10% FIG. 18. Total pressure index percentage in lame dogs shows a significant difference between injured and contralateral limb FIG. 18. That statistical difference was no longer evident in post-treatment group. Similarly, after treatment, ultrasonography showed normal homogenous fiber pattern in the injured tendon in 90% of cases, with the remaining 10% of cases showing improvement. Injured supraspinatus cross sectional area was significantly higher than normal contralateral tendon on initial examination. Post-treatment with ASC/PRP, that enlargement was no longer evident at all time points FIG. 19. FIGS. 17A-17C show ultrasonographic images of normal supraspinatus tendon (FIG. 17A), injured (contralateral to the normal supraspinatus tendon) supraspinatus tendon (FIG. 17B), and healing of the injured supraspinatus tendon four months post adipose stem cell/PRP treatment as described in this Example (FIG. 17C). The linear fiber pattern having long echogenic lines running parallel with the long axis of the tendon can be imaged upon longitudinal alignment with the tendon was observed and is thought to be a result of the linear organization of the collagen fibers of the tendon.

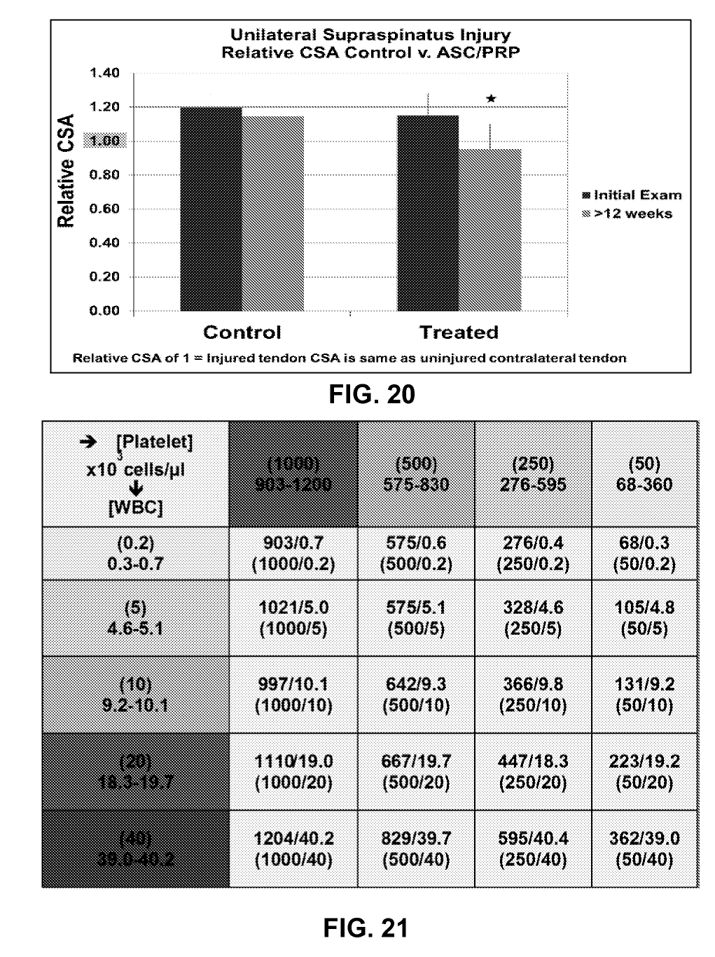

Furthermore, in a small pilot study comparing cross sectional area of ASC/PRP treated to 5 untreated tendons, normalizing injured tendon cross-sectional area to contralateral uninjured tendon to account for size variation of canine patients (normal=1.00) showed that untreated injured tendons were significantly larger than contralateral tendons, and that they did not improve with conservative management after 12 weeks (FIG. 20). In contrast to this, ASC/PRP treated tendons became significantly smaller after treatment, and approached a mean relative CSA of 1.00, which indicates the injured tendon is the same size as contralateral uninjured tendon.

### Example 2

### Optimized Platelet Rich Plasma

Introduction

Platelet rich plasma is at the forefront of regenerative therapies in its ability to partially or fully restore the normal functioning of vital tissues and organs in the body.

Autologous preparations do not require FDA approval, and can be administered within minutes of blood collection. Moreover, as opposed to conventional surgical treatment options, platelet rich plasma therapy is minimally invasive and preparation doesn't require sophisticated lab equipment and reagents. In the veterinary community, specialists across a range of species including dogs, horses and humans have demonstrated the regenerative efficacy of PRP in a number of clinical applications, mostly pertaining to orthopedic injuries.

Platelet-rich plasma is prepared in vitro from blood and consists of a concentrate of platelets suspended in blood plasma, generally five times the average blood platelet concentration. Leukocytes, in small proportions are an inevitable component of most preparations, due to the proximity of the buffy coat to the platelet-rich layer in blood after centrifugation. However, whether their presence in the plasma constitutes a bane or a boon in terms of its therapeutic efficacy, is a widely debated topic amongst medical practitioners.

In humans, platelet rich plasma has varied applications in the fields of dentistry, orthopedics, and sports medicine, and trauma surgery. Platelet rich plasma has been previously used to treat intralesional injuries, tendinopathies and intraarticular defects in horses. Of particular importance in athletic horses, are tendon and ligament injuries. The nature of these injuries make them more susceptible to recurrence, and there are not many treatment options currently in the market that promise a long term cure, making platelet rich plasma a valuable treatment option.

Modifications of a platelet and leukocyte concentrate, derived from blood, have been prepared previously and include pure platelet-rich or leukocyte rich plasma, platelet rich fibrin and platelet and leukocyte rich fibrin. Various forms of administration of platelet-rich plasma include a gel for topical applications and liquid injections tor arthroscopic applications. Platelet concentrates are also injected into 3D scaffold implants.

Platelet rich plasma is prepared under sterile conditions via both proprietary and commercially available methods. Variability's in the cell and protein content of platelet-rich plasma can be attributed to differing initial blood volumes and preparation techniques. Previous research demonstrates that different clinical applications favor different preparation protocols and PRP compositions. Certain in-vivo applications favor leukocyte-rich PRP whereas others such as the treatment often tendinopathy are biased towards the complete removal of leukocytes from PRP. In terms of equine tendon and ligament injuries, there is currently no established standard for PRP treatment. There is an ongoing debate on whether platelets, leukocytes or a definite ratio of platelets and leukocytes in plasma are more beneficial in terms of the healing potential of PRP.

The rationale for using PRP as a therapy can be attributed to the presence of numerous anabolic growth factors, including but not limited to platelet-derived growth factor, fibroblast growth factor, insulin-like growth factor, vascular endothelial growth factor and transforming growth factor. These growth factors promote cell growth and localization at the site of injury, cell adhesion, reconstruction of the extracellular matrix and tissue-specific cell differentiation. When platelet rich plasma is injected into a site of injury, platelets in the plasma become activated in response to chemical stress signals from the surrounding environment. Activation of platelets can be either endogenous or induced exogenously. Exogenous activation involves the use of external activation factors such as calcium, adenosine triphosphate, thrombin and collagen. Platelet activation is characterized by clotting and degranulation of platelets, followed by the release of growth factors into the plasma. More than 95% of these pre-synthesized growth factors are released by alpha granules within an hour of platelet clotting, followed by indefinite additional synthesis by degranulated platelets. Residual leukocytes present in the plasma are believed to contribute to the catabolic pool of cytokines including interleukin-1 beta and tumor necrosis factor-alpha, however previous research demonstrates that they also have beneficial anti-infectious and antimicrobial properties.

The objective of this study was to determine the inter-relationships between cell and protein content in a proprietary preparation of platelet-rich plasma, to develop an optimized formulation for subsequent in vitro studies using freshly harvested ligament specimens from horses. In this study, we have used a proprietary protocol for PRP preparation, designed specifically for equine tendon and ligament injuries. A total of twenty formulations of platelet-rich plasma varying concentrations of platelets and leukocytes were developed for this purpose, with the goal of finding the optimal platelet to leukocyte ratio which had the maximum amount of beneficial growth factors and a minimal amount of inflammatory mediators. We hypothesize that PDGF and TGF arc derived from platelets and any increase in the concentration of platelets would result in a direct increase in the production of these growth factors, regardless of the leukocyte concentration. However, the concentration of inflammatory mediators is directly correlated to leukocytes in plasma. Based on the results of the study we aim to determine three of the best formulations of platelet-rich plasma from our different sets of ratios for phase 2 testing.

Methods

Research Subjects: Five healthy research horses with no evident abnormalities, participated in this study. Three liters of whole blood were collected aseptically from each horse into sterile blood bags containing the anticoagulant CPDA-1 (Terumo corporation, Cat.no. BB*SCD456A), in accordance with Virginia Tech's Institutional Animal Care and Use Committee protocol.

Blood Collection and Processing: PRP was generated in a similar fashion as detailed in Example 1. The volumes used were different to account for the size difference between dogs and horses. Briefly, collected blood was transferred to 200 ml conical tubes and centrifuged at 800 g for 10 minutes with refrigeration. Platelet rich plasma from the top layer was carefully aspirated, leaving the middle layer of buffy coat untouched. Enough whole blood was centrifuge to allow for at least 4 mL of PRP. For horses, about 4 mL of PRP per injection was needed. The plasma layer was then centrifuged a second time at about 3000 g for 10 minutes, to concentrate the platelets. The resulting plasma fraction was platelet deficient and was referred to as platelet poor plasma (PPP) Most of the PPP from the second spin was removed and saved in 50 ml collection tubes, and the remaining pellets were resuspended in a volume of remaining PPP to yield a platelet concentrate.

Unlike the PRP of Example 1, in this Example the buffy coat from the first centrifugation step was carefully aspirated and centrifuged a second time at 800 g for 10 minutes to remove any residual plasma and yield a white cell concentrate. Cell quantification of whole blood, PPP, platelet concentrate and white cell concentrate fractions was performed using an automated cell counter. This step can also be performed on canine or human blood samples.

Sample Preparation and Activation: Based on the initial cell counts, 50 ml suspensions with each of the following concentrations of platelets and WBCs were prepared and mixed together: 1,000, 500, 250, and 50×103/μl platelets, and 40, 20, 10, 5 and 0.2×103/μl white blood cells. Thus, for the preparations with 1000×10^{3/μ}l platelets, there were 5 PRP samples with each of 40, 20, 10, 5, and 0.2×103/μl WBCs, to yield a total of 20 different formulations of PRP varying concentrations of platelets and white blood cells. See Table 1. Additionally, the whole blood, platelet poor plasma, platelet concentrate, and white blood cell concentrate fractions were used as controls in the study.

Cell counts of the different PRP formulations were re-confirmed using an automated cell counter. PRP was activated overnight in glass tubes containing 200 μl of calcium chloride (10%), kept at 37 degrees Celsius. The next day, the vials were centrifuged at 3000 g for about 5 minutes to extract the conditioned serum from the clots. Clots were stored in 0.5% triton-X 100. Protease inhibitor was supplemented to both serum and clot aliquots, which were then stored at −80 degrees Celsius.

TABLE 1
Ratios of Platelet:WBC

WBC count (×10³ cells/μL)

| | | | | |
|---|---|---|---|---|
| Platelet Count | 1000:40 | 500:40 | 250:40 | 50:40 |
| (×10³ cells/μL) | 1000:20 | 500:20 | 250:20 | 50:20 |
| | 1000:10 | 500:10 | 250:10 | 50:10 |
| | 1000:5 | 500:5 | 250:5 | 50:5 |
| | 1000:0.2 | 500:0.2 | 250:0.2 | 50:0.2 |

Evaluation of Platelet Resting Status: PRP formulations required platelets to be in the rested state in the absence of platelet activating factors. To counter the possibility of platelet activation due to centrifugation and sedimentation, plasma solutions were visually examined for changes in consistency after being activated. Further, control and activated cell populations were washed and stained with fluorescein-conjugated, mouse monoclonal antibodies to the platelet-specific surface antigen P-selectin (Human P-Selectin/CD62P FITC MAb, Mouse IgGJ, R&D systems, Cat.no.BBA34), and analyzed by flow cytometry.

Enzyme-linked Immunoassay: The frozen serum aliquots were assayed for growth factors and inflammatory mediators by commercially available ELISA kits. Platelet derived growth factor(PDGF)-BB levels in the serum were quantified using the Human PDGF-BB Quantikine Elisa kit by R&D systems Inc (Cat.no.DBBOO), transforming growth factor (TGF) beta 1 was quantified using the TGF beta 1 Emax immunoassay system by Promega Inc. (Cat.no.G7591), insulin like growth factor was quantified using the Non-extraction IGF-1 Elisa by Beckman Coulter Inc. (Cat.no. IX4L-1 0-2800), stromal cell derived growth factor was measured using the Human CXCI 12/SDF-1 alpha Quantikine ELISA Kit by R&D systems Inc. (Cat.no.DSAOO), interleukin-1 (IL 1 or IL-1)) beta was measured using the Equine IL-I beta E11SA VetSet by Kingfisher Biotech Inc. (Cat. no. VSOJ31E-002), and interleukin-1 receptor antagonist protein was measured using the Equine IL-Ira/IL-JF3 DuoSet by R&D systems Inc. (Cat. no. DY2466), according to the manufacturers protocol.

Sample dilution was optimized to obtain a limit detection within a linear range. Standard curves were prepared for each assay as per the kit protocols and unknowns were extrapolated from the curve. To assay fibroblast growth factor (FGF)-2, a primary rabbit polyclonal IgG antibody at a 1:30 dilution (Santacruz Biotechnology, Inc., Cat.no. sc-79) and a goat antirabbit IgG-HRP secondary antibody at a 1:2000 dilution (Santacruz Biotechnology, Inc., Cat.no.sc 2004) were paired together in an inverted ELISA fom1at as per the protocol on the company website. A commercially available FGF-2 peptide standard (Santacruz Biotechnology Inc., Cat.no. sc-79P) was also included in the assay, to plot a standard curve.

Results

Analysis of Platelet Resting Status: Visual examination of plasma solutions after activation with glass and calcium chloride, revealed evidence of platelet clotting, which was indicative of activated cells. Inactivated plasma was in the form of a uniform suspension of platelets, devoid of clots. Further, several procedural centrifugation steps of the initial blood sample did not change the consistency of plasma solutions nor did it induce platelet clumping, indicative of platelets in their rested state.

To further analyze morphological changes in the platelets, control (unactivated) and activated platelet populations were washed and stained with fluorescein-conjugated primary antibodies to P-selectin, which is a biomarker that only expresses itself on the surface of activated platelets. Single-parameter histogram analysis by flow cytometry revealed a distinct separation of the activated cell populations from the unactivated groups.

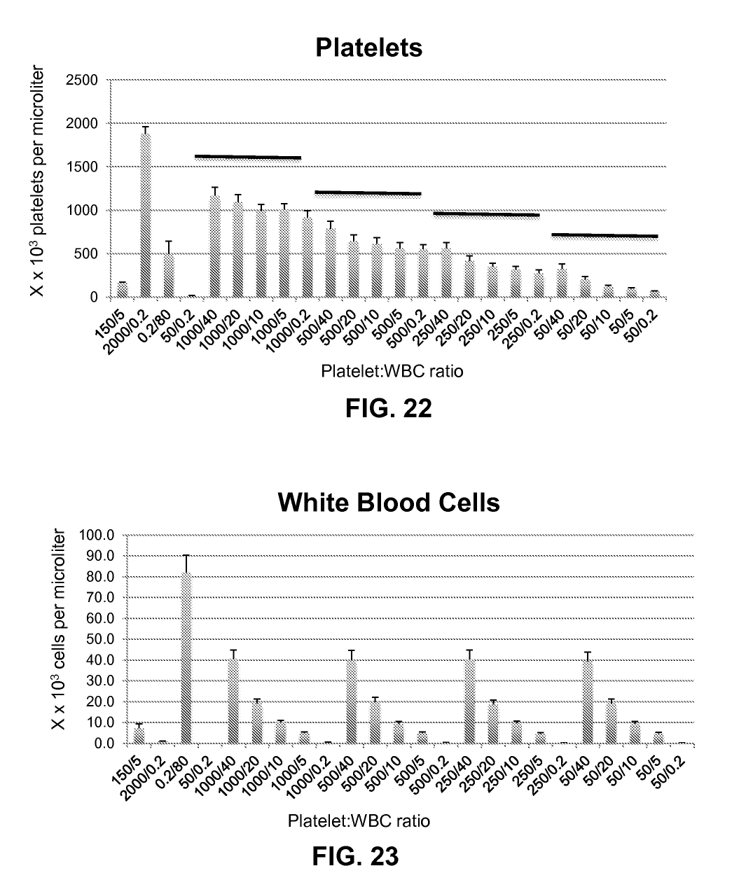

Quantitation of Cell and Protein Content in Plasma: Results of the quantitation of cell and protein content in the prepared PRP with varying platelet:WBC ratios are demonstrated in FIGS. 21-29. FIG. 21 shows a table depicting the approximate concentrations of platelets and white blood cells in the 20 different formulations of PRP, measured using an automated cell counter. FIGS. 22-23 show graphs demonstrating the graded concentrations of platelets and white blood cells, respectively. FIG. 24 shows a graph demonstrating platelet derived growth factor (PDGF) levels in the PRP compositions. PDGF values were directly correlated to platelet number (FIG. 22). Lowest values of PDGF were detected in the 50×10³ platelets/μl range and greatest values of PDGF were detected in the 1000×10³ platelets/μl range. Amongst the assay controls, the highest values were measured in the concentrated platelet and platelet rich plasma fractions, and the lowest were measured in the concentrated white blood cell and whole blood fractions.

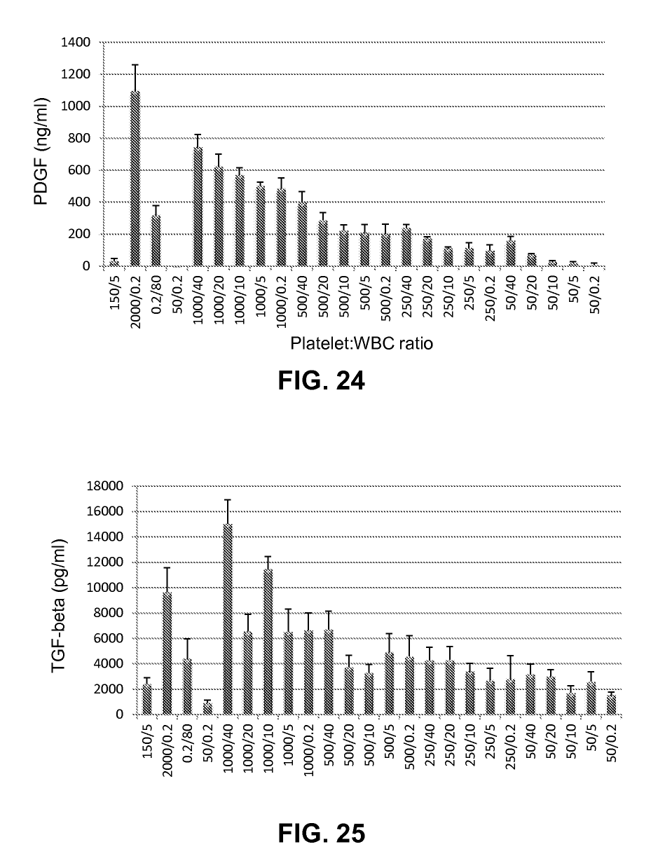

FIG. 25 shows a graph demonstrating transforming growth factor (TGF)-beta levels in PRP compositions. TGF-beta levels were observed to be more variable within a range but followed a similar pattern of direct correlation to platelet number across ranges. Amongst the assay controls, the whole blood and white cell concentrate fractions measured the lowest quantities of TGF in comparison with the platelet rich fractions.

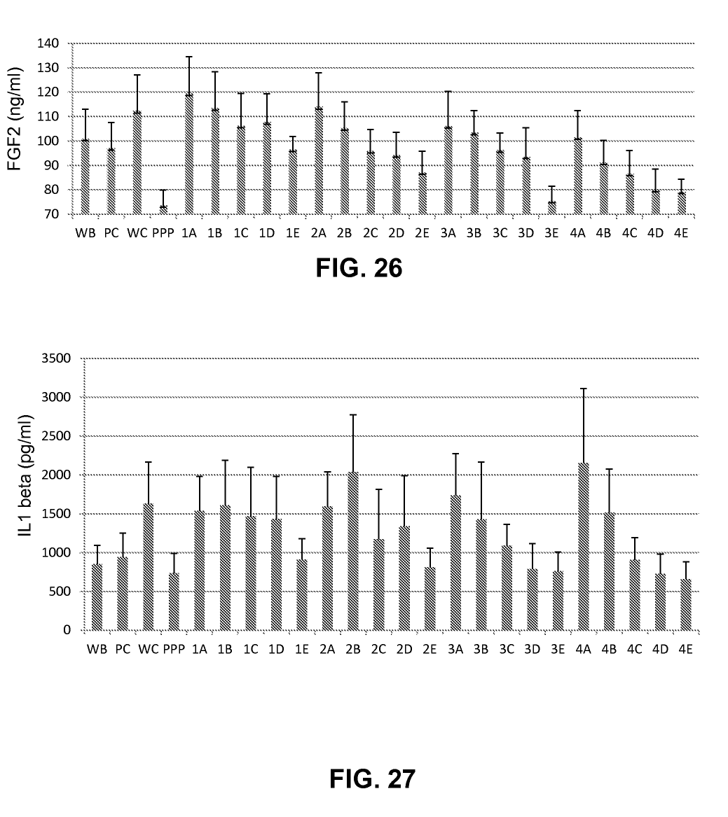

FIG. 26 shows a graph demonstrating fibroblast growth factor-2 levels in PRP compositions. FGF2 levels across the groups were more closely related to leukocyte levels in plasma, with greater FGF-2 detected in PRP containing higher levels of leukocytes and lower FGF2 detected in PRP containing lower levels of leukocytes. The correlation to platelets was less significant with regards to FGF-2.

FIG. 27 shows a graph demonstrating interleukin-1 (IL 1) beta levels in PRP compositions. Levels of detection were variable amongst the groups with higher platelet to white blood cell ratios. However, groups with lower ratios of platelets to white blood cells display a strong correlation to leukocytes in plasma.

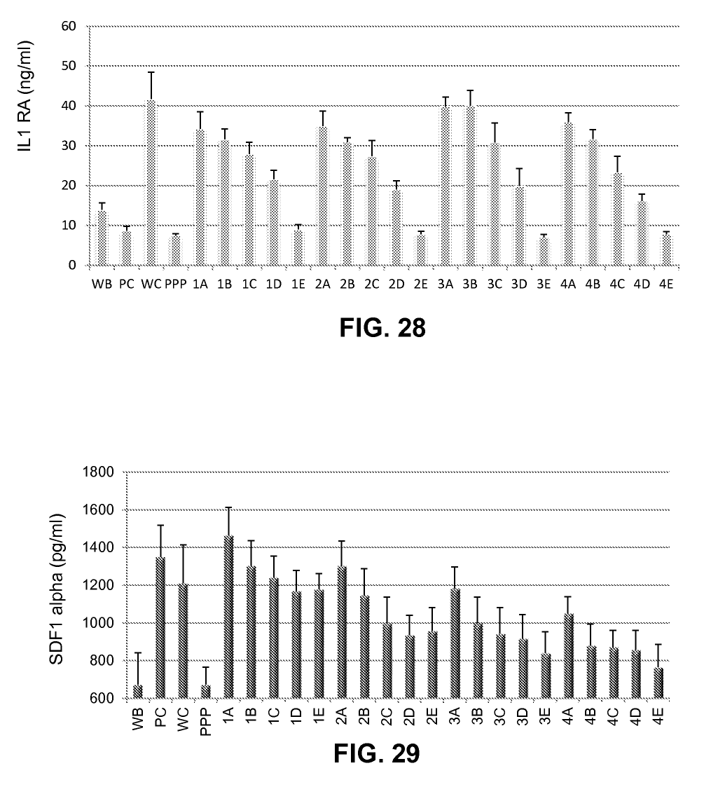

FIG. 28 shows a graph demonstrating interleukin-1 (IL 1) receptor antagonist protein levels in PRP compositions. Receptor molecules in the different formulations are strongly correlated to leukocyte levels in plasma. In comparison to the IL 1-beta levels, much lower quantities of its receptor were detected.

FIG. 29 shows a graph demonstrating stromal cell derived growth factor alpha in PRP compositions. SDF quantities across the PRP groups were strongly correlated to both platelet and leukocyte levels in the same. Higher platelet to leukocyte ratios have higher quantities of the growth factor in comparison with lower ratios. Greater leukocyte levels in plasma can be indicative of greater quantities of the growth factor.

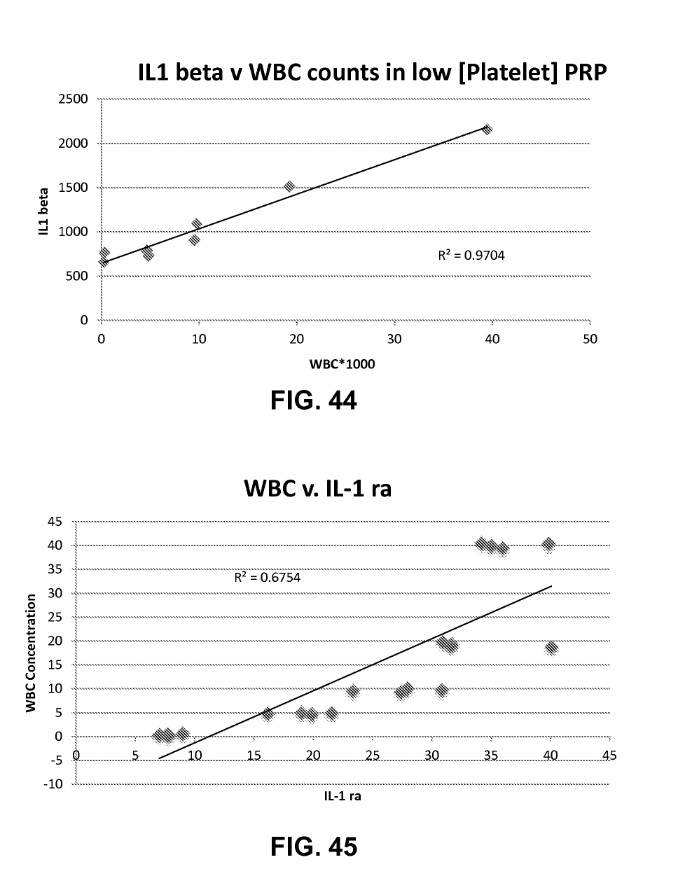

FIG. 44 shows a graph demonstrating IL 1-beta versus WBC counts in low platelet concentration [platelet] PRP. It was observed that when the PRP is 2× or lower concentrated for platelets, an increased WBC concentration was correlated with high IL 1-beta (R2=0.9704).

FIG. 45 shows a graph demonstrating the correlation between WBC concentration and IL-RA levels.

### Example 3

### Autologous Conditioned Serum Preparation

Autologous conditioned serum (ACS) has been prepared from equines and canines according to the following protocol. ACS has been used as a diluent for stem cells used in soft tissue injury treatment, such as treatments for tendinopathy. ACS has been prepared by obtaining previously prepared and unactivated PRP with amikacin. About 2 mL of ACS was prepared per lesion for dogs and about 4 mL of ACS was prepared per lesion for horses. Sterile glass beads were obtained and placed in a conical tube. For ACS, 10 sterile beads (e.g Zymo Research Corporation Rattler Plating Beads Cat. No. 50-444-634) were used per 5 mL (or less) of PRP. 150 μL of filtered, room temperature 10% CaCl₂ (100 g/ml) per 5 mL of PRP was added to the sterile glass beads. For other amounts of PRP, a ratio of 30 μL of 10% CaCl₂ per 1 mL of PRP was used. Any pellet that may have formed in the PRP with amikacin was gently resuspended being careful to avoid aspirating the pellet or creating bubbles that will trap the platelets. About 5 mL (or other amount as necessary) of the PRP was added to the tube containing the sterile glass beads and CaCl₂.

The contents of the tube(s) was agitated gently to mix. After mixing, the mixture was incubated at about 37° C. In some cases the tubes were placed on their sides to increase the glass surface area, which can enhance clotting. In some instances, clotting was observed to begin within 30 minutes. In cases where clotting had not begun within 30 minutes, an additional 100 μL of 10% CaCl₂ was added to the mixture. Incubation continued for about 2-3 hours (including the first 30 minutes) or until clot retraction was maximized. In some cases this can be overnight (about 12-16 hours). After clot formation, the remaining serum, which was the ACS, was ascetically removed a filtered through a 0.2 mm syringe filter using an 18 gauge needle. ACS was used immediately or stored for later use at −80° C.

The ACS can be used as diluent for the delivery of stem cells.

### Example 4

### Preparation and Use of Tendon Precursor Cells (TPCs) Introduction

Tendon injuries are a significant cause of morbidity in equine performance horses.

Superficial digital flexor tendon (SDFT) injury is reported to represent up to 43% of overall Thoroughbred racehorse injuries leading to early retirement of approximately 14% of horses. Natural repair is slow and results in inferior structural organization and biomechanical properties, therefore, reinjury is common with rates of up to 80% reported in racehorses. The inability of tendon to regenerate after injury, or heal with mechanical properties comparable to the original tissue, is likely attributable to low vascularity and cellularity of the tissue, low number of resident progenitor cells, and healing under weight-bearing conditions.

Strategies to improve tendon healing have focused on enhancing the metabolic response of tenocytes, modulating the organization of the newly synthesized extracellular matrix, or administering progenitor cells to enhance repair. Significant research effort has been directed at the use of adult mesenchymal stem cells (MSCs) as a source of progenitor cells for equine tendon repair. Recent clinical applications have utilized adult autologous

MSCs derived either from adipose tissue or bone marrow aspirates. Isolation of a homogeneous population of progenitor cells from bone marrow is time-consuming, and there is much variation in cell numbers, cell viability and growth rates among samples. Recently, a population of progenitor cells with multidifferentiation potential has been isolated from equine flexor tendons providing an alternative source of progenitor cells as well as a target cell for therapeutic intervention.

Tendon is composed primarily of type I collagen arranged into fibers aligned along the longitudinal axis of the tendon. Collagen type III is also present but only comprises approximately 4-5% of total collagen in the metacarpal region of normal adult equine SDFT. Cartilage oligomeric matrix protein (COMP), and decorin are important extracellular matrix components produced by tenocytes, that together with collagen type III, have been shown to be integral in the regulation of fibrillogenesis and organization of tendon. Collagen fibers are surrounded by ground substance composed of proteoglycans and glycosaminoglycans (GAGs) that help package the collagen fibrils. GAGs are negatively charged macromolecules, that are important in determining the water content of the extracellular matrix (ECM) of tendon. Collagen, COMP and proteoglycan synthesis are all increased following tendon injury.

The interaction between cells and the ECM is an important regulatory factor of cell function. Proliferation, migration, differentiation and gene expression of many cell types may all be altered by adhesion to and interaction with matrix proteins and the extracellular environment. Tendon progenitor cells reside within a niche comprised primarily parallel collagen fibers that plays an important role in regulating their function and differentiation. Two independent studies have evaluated the effects of acellular native tendon matrices on equine tenocytes (TCs) and BMMSCs, or TPCs and BMMSCs. Both demonstrated engraftment and alignment with the highly organized collagen network. Positive effects of collagen type I-coated surfaces on BMMSC proliferation and gene expression of collagen types I and III, fibronectin, and decorin were reported; however, the effect of collagen on TPC proliferation has not been studied. It is unknown whether a collagen-rich extracellular environment could influence TPC growth and whether there are any differences between species-specific type I collagens in their ability to influence cell proliferation and tendon matrix gene expression.

Some objectives of this Example were to compare cell growth kinetics and tendon matrix component biosynthetic capabilities of TPCs and BMMSCs cultured on commercially available bovine, porcine and rat type I collagen sources. It was hypothesized that a randomly oriented collagen matrix would preferentially support TPC proliferation versus BMMSCs, and upregulate tendon-related gene expression and therefore provide a culture system and progenitor cell type with advantages over the current practice of BMMSC expansion on standard tissue culture surfaces. A culture system that is able to efficiently provide adequate cell numbers for cell therapy and direct progenitor cells to produce tendon matrix would be beneficial to regenerative medicine efforts to improve the outcome of equine flexor tendon injury.

Materials and Methods

Collection of samples: Bone marrow aspirates and tendon samples were collected aseptically from six young horses (2-5 years) euthanatized for reasons unrelated to musculoskeletal disease. Samples were obtained in accordance with the guidelines reviewed and approved by the Institutional Animal Care and Use Committee of the Virginia Polytechnic Institute and State University. All horses were sedated with 0.5-1.0 mg/kg of xylazine intravenously prior to induction of anesthesia. Anesthesia was induced with 2.2 mg/kg of ketamine and 0.1 mg/kg of diazepam given intravenously. General anesthesia was maintained by intravenous infusion of 5% guaifenesin, 1 mg/mL ketamine and 1 mg/mL of xylazine. Following collection of bone marrow aspirates as previously described(Fortier L A, Nixon A J, Williams J, et al. Isolation and chondrocytic differentiation of equine bone marrow-derived mesenchymal stem cells. Am J Vet Res 1998;59:1182-1187), all horses were euthanatized with 104 mg/kg of pentobarbital sodium given intravenously. The tendon specimens were collected immediately following euthanasia, as detailed below.

Cell culture technique: All cell cultures (both BMMSCs and TPCs) were incubated at 37° C. in a 5% carbon dioxide atmosphere with 90% humidity for media supplementation every 48 hours. Once approaching 70% confluence, adherent cells were trypsinized using standard tissue culture technique, counted and plated at 500,000 cells per 75- cm2 flasks to propagate adequate cell numbers. Time to confluence and cell counts at trypsinization were recorded. BMMSCs were grown in BMMSC medium: low-glucose Dulbecco's modified eagle medium (DMEM)' supplemented with 10% fetal bovine serum (FBS)“, 300 μg of L-glutamine/mL, 100 U of sodium penicillin and 100 μg of streptomycin sulfate”/mL. TPCs were grown in TPC medium: high-glucose DMEM supplemented with 10% FBS, 10% Horse Serum (HS), 37.5 μg/ml of ascorbic acid, 300 μg of L-glutamine/mL, 100 U sodium penicillin and 100 μg of streptomycin sulfate /mL. TPCs and BMMSCs were each tested for cell proliferation in both DMEM glucose concentrations and both serum concentrations (low-glucose DMEM v. high-glucose DMEM; 10% FBS v. 10% FBS 10% HS), and the above media were the optimal media tested for each cell type (data not shown).

Processing of bone marrow mesenchymal stem cells: The left tuber coxae was clipped, aseptically prepared and a bone marrow biopsy needle^{v} was used to aspirate a total of 60 mLs of bone marrow into 2 syringes each containing 5,000 units of heparin diluted to a volume of 10 mLs with phosphate buffered saline (PBS). Bone marrow aspirate was then transferred to centrifugation tubes, diluted with PBS solution (2:1) and centrifuged at 300× g for 15 minutes at 4° C. The cell pellets were resuspended in PBS solution, and centrifugation repeated. Pelleted cells were resuspended in 12 mL of BMMSC medium in 75-cm² flasks. Processing of tendon-derived progenitor cells: The entire metacarpal SDFT was harvested aseptically from both forelimbs from each horse following euthanasia. A 2-cm³ sample from the mid-metarcarpal core region was snap-frozen in liquid nitrogen for control RNA isolation. A 6-cm×1-cm² sample of tendon from the mid-metacarpal tensional region was diced into 0.5-cm³ pieces and digested in an orbital shaker for 16 hours at 37° C. in 0.1% collagenase^{vi} high-glucose DMEM supplemented with 1% FBS, 37.5 pg/mL of ascorbic acid^{vii}, 100 U of sodium penicillin and 100 μg of streptomycin sulfate /mL. Following digestion, the suspensions were passed through 100 μm sterile cell filters^{viii}. The isolated cells were collected by centrifugation at 300× g for 5 minutes. The supernatant was removed, and the cell pellet was resuspended in TPC medium. The cells were then subjected to a differential adherence protocol as previously described (Stewart A A, Barrett J G, Byron C R, et al. Comparison of equine tendon-, muscle-, and bone marrow-derived cells cultured on tendon matrix. Am J Vet Res 2009;70:750-757 and Barrett J G, Stewart A A, Yates A C, et al. Tendon-derived progenitor cells can differentiate along multiple lineages. Vet Orthop Soc Conf 2007;34:56). Briefly, cells were plated and allowed to settle undisturbed for 2 days prior to the slowly adherent cells being removed and placed in a new tissue culture plate. The slowly adherent cell population, or TPCs, was expanded to obtain adequate numbers for experiments, all experiments used cells from passage 1. The isolated TPCs were cultured in 75-cm² flasks in TPC medium as described above until approximately 80% confluence. Time to confluence and cell counts at trypsinization were recorded. Cells were released from the flasks with trypsin (0.05%) and re-seeded at 5000 cells/cm². TPC characterization will be published elsewhere; however, TPCs stain 80% with anti-CD90 antibody, 40% with anti-CD44 antibody, and comprise a mixture of cells, some of which can differentiate toward adipose, cartilage and bone (data not shown). Barrett J G, Stewart A A, Yates A C. Tendon-derived progenitor cells can differentiate along multiple lineages. (Annual Conference Veterinary Orthopedic Society 2007).

Tendon progenitor cell and bone marrow mesenchymal stem cell culture- Once adequate cell numbers for each TPC and BMMSC culture were obtained, cells were trypsinized and resuspended at a concentration of 1.5×10⁷ cells in 1.5 mL of DMEM, 10% FBS, and 10% DMSO, and then stored in the vapor phase of liquid nitrogen. All cultures that were utilized for these experiments were from passage 1. The viability of all cryopreserved cells was assessed with trypan blue stain^{ix} immediately after thawing.

First passage TPCs and BMMSCs were seeded at 1×10³ cells/cm² in 24-well plates, and 25 cm² (T25) flasks. For the well plates and T25 flasks, experiments were equally divided between surfaces with no modification and wells and flasks pre-coated with bovine^{x}, highly purified bovine^{xi}, porcine^{xii}, and rat^{xiii} collagen type I. Tissue origins for each collagen preparation were as follows: bovine: dermis, highly purified bovine: tendon, porcine: dermis, and rat: tendon. The porcine and rat collagens were dissolved in 0.02M acetic acid, and the bovine and highly purified bovine collagens were dissolved in 0.01M HCl. Diluted collagen solution was added to tissue culture surfaces to result in a final surface area concentration of about 8 μg/cm² of the respective collagen, and washed with PBS to normalize pH. Experiments performed on twenty four-well plates were performed in triplicate and T25 flasks for mRNA analysis were performed in duplicate. Media was changed about every 48 hours and cultures were monitored daily over the 7 day culture period. Photomicrographs were taken on about day 5.

Cell proliferation—The CellTiter 96 Aqueous^{xiv} assay was used to determined cell number of 3 replicates of each cell type and collagen group on 24-well plates on days 4 and 7. For the tissue well plates, about 50 μL of the CellTiter reagent was added to fresh ascorbate-free media in each well and the cells were incubated at about 37° C. for about 2.5 hours. About 100 μL of a sample of media from each test well were transferred to a 96-well plate and absorbance was measured at 490 nm in a microplate reader^{xv}. All samples were assayed in triplicate, and a mean value was calculated to provide a single data point. The optical density values were converted to a cell number by reference to standard curves carried out on cells from each horse for each cell type. Specifically, the standard curve was generated by trypsinizing cells and counting using a hemacytometer. Cells were then plated as a serial dilution in 24-well plates, and the same procedure was performed on the standard curve wells as the sample wells after the cells equilibrated overnight.

RNA isolation and gene expression-The expression of selected genes characteristic of tendon fibroblast phenotype (collagen types I and III, COMP, decorin) was quantified on day 7 by real-time PCR. Duplicate 25-cm² flask samples from each experimental group were isolated using Trizol^{xvi} method, and purified by use of a commercially available column-based protocol^{xvii}. This protocol included an on-column

DNase treatment to exclude genomic template contamination. The RNA from freshly collected, snap-frozen tendon was used as a reference control for gene expression analysis, to relate in vitro expression levels to in vivo expression.

Tendon tissue RNA was isolated by use of a protocol adapted from a technique for cartilage RNA isolation (Stewart M C, Saunders K M, Burton-Wurster N, et al. Phenotypic stability of articular chondrocytes in vitro: the effects of culture models, bone morphogenetic protein 2, and serum supplementation. J Bone Miner Res 2000;15:166-174).

Briefly, tissues were pulverized under liquid nitrogen, then homogenized in guanidium isothiocyanate lysis buffer, extracted with phenol-chloroform, precipitated with isopropanol, and purified by use the column-based protocol (above).

One microgram of RNA in each sample was converted to cDNA with a commercial transcription kit and oligo (dT) primers^{xviii}. Target cDNAs were amplified via real-time PCR by use of Taq DNA polymerase (TaqMan®)^{xix} and gene specific primers and MGB probes from available published equine sequences demonstrated in Table 2 below. Real time quantitative PCR assay was performed in triplicate for collagen type I, collagen type III, COMP, and decorin and as a reference, 18S RNA. A Real-Time PCR system^{xx} was used to perform the assay. All reactions were run as single-plex, and the relative gene expression was quantified by use of the 2^{-ΔΔCt} method (Livak K J, Schmittgen T D. Analysis of relative gene expression data using real-time quantitative PCR and the 2(-Delta Delta C(T)) Method. Methods 2001;25:402-408). Collagen type I, collagen type III, COMP and decorin mRNA values were normalized to expression of 18S RNA, and subsequently normalized to tendon tissue expression of each gene product of interest.

TABLE 2

| Gene of | Forward | Reverse | |
|---|---|---|---|
| Interest | Primer | Primer | Probe |
| Collagen | GCCAAGAAGAAG | TGAGGCCGTC | ACATCCCAG |
| Type I | GCCAAGAA | CTGTATGC | CAGTCACCT |
| Collagen | CTGCTTCATCCC | ATCCGCATAG | AACAGGAAG |
| Type III | ACTCTTATTCT | GACTGACCAA | TTGCTGAAG |
| COMP | GAGATCGTGCAA | GACCGTATTC | CTGGCTGTG |
| | ACAATGAAC | ACGTGGAACG | GGTTACA |
| Decorin | AAGTTGATGCAG | GGCCAGAGAG | ATTTGGCTA |
| | CTAGCCTGA | CCATTGTCA | AATTGGGAC |
| 18S RNA | GAGGCCCTGTAA | CGCTATTGGA | CAAGTCTGG |
| | TTGGAATGA | GCTGGAATTA | TGCCAGCA |

Glycosaminoglycan—Cell monolayers were collected for quantification of glycosaminoglycan production on day 7. Cell monolayers were released with 2 mM EDTA at 37° C. for 10 minutes, and digested in papain^{xxi} (0.15 mg/mL) at 65° C. overnight. The 1,9-dimethymethylene blue assay was performed by use of the direct spectrophotometric method to measure the total GAG content (Oke S L, Hurtig M B, Keates R A, et al. Assessment of three variations of the 1,9-dimethylmethylene blue assay for measurement of sulfated glycosaminoglycan concentrations in equine synovial fluid. Am J Vet Res 2003;64:900-906 and Farndale R W, Buttle D J, Barrett A J. Improved quantitation and discrimination of sulphated glycosaminoglycans by use of dimethylmethylene blue. Biochim Biophys Acta 1986;883:173-177. Results were compared with a chondroitin sulfate standard curve and standardized to relative cell number (DNA). Total DNA content was determined from the papain digest by use of fluorometric dye assay^{xxii} and a microplate reader, as previously described (Kim Y J, Sah R L, Doong J Y, et al. Fluorometric assay of DNA in cartilage explants using Hoechst 33258. Anal Biochem 1988;174:168-176). Results were compared with a standard curve of calf thymus DNA.

Statistical Analysis—All cell count data were logₑ transformed to obtain normal distribution. Differences of cell count, GAG synthesis and gene expression between collagen groups and cell type, were evaluated by use of mixed-model repeated measures ANOVA. Pair-wise comparisons were made on significant differences identified with ANOVA using Tukey's post hoc test. A commercial statistical program^{xxiii} was used to perform analysis. Cell count data are reported as geometric least squares means and relative gene expression data are presented as mean±SD. Values of P≦0.05 were considered significant.

Results

Cell isolation and expansion: Following differential adherence plating, the tendon progenitor cells proliferated in uniform monolayer cultures and adopted a tightly packed, fusiform morphology. BMMSCs grew in clonal expansion groups that had focal areas of tightly packed cells with fusiform morphology. Less time to confluence after initial plating was recorded for TPCs than BMMSCs (5-8 days and 12-14 days, respectively) but thereafter, subsequent passage times for both cell types were similar (4-6 days).

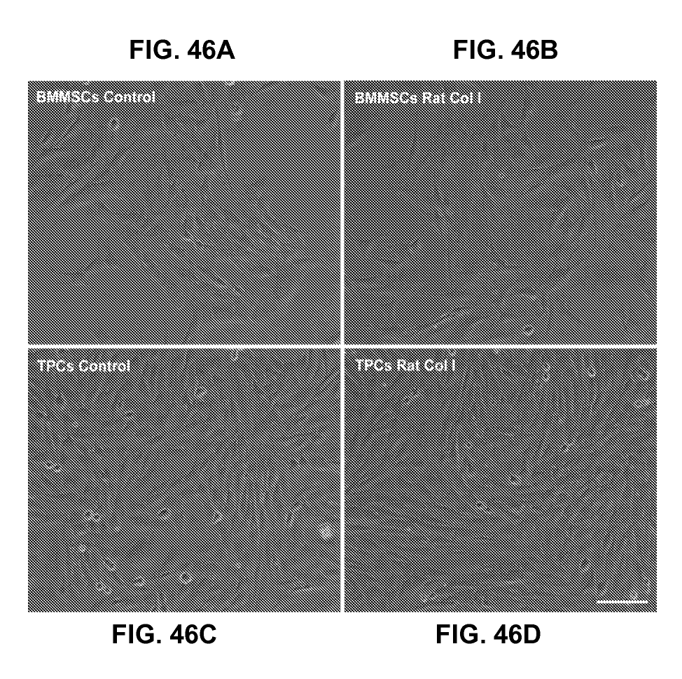

Cell morphology and number. FIGS. 46A-46D show representative images of the TPCs and BMMSCs growing on control (uncoated) wells v. collagen-coated wells on day 5. Cell morphology after 5 days in culture on collagen coated plates was not subjectively different; however, a difference in cell number between cultures is apparent.

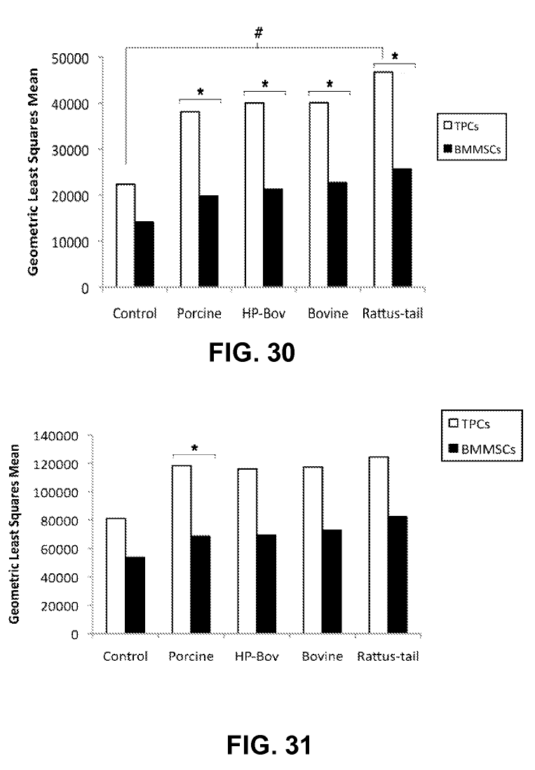

Increased cell growth was observed on all collagen coated plates for both BMMSCs and TPCs versus control on days 4 and 7; however, only TPCs cultured on rat collagen was significantly (P=0.05) increased on day 4 (FIGS. 30-31 and 39). When comparing between cell type, TPCs cultured on all collagen groups yielded significantly more cells than similarly cultured BMMSCs on day 4, but only when cultured on porcine collagen-coated surfaces on day 7. FIGS. 30-31 show graphs demonstrating cell number of TPCs and BMMSCs following 4 (FIGS. 30) and 7 (FIG. 31) days of culture on collagen groups. # indicates statistical significance (P˜0.05) for cell type between collagen group and * between TPCs & BMMSCs within collagen group. FIG. 39 shows a table demonstrating the cell number geometric 95% confidence interval for collagen groups for TPCs and bone marrow mesenchymal stem cells (BMMSCs) on days 4 and 7 of culture.

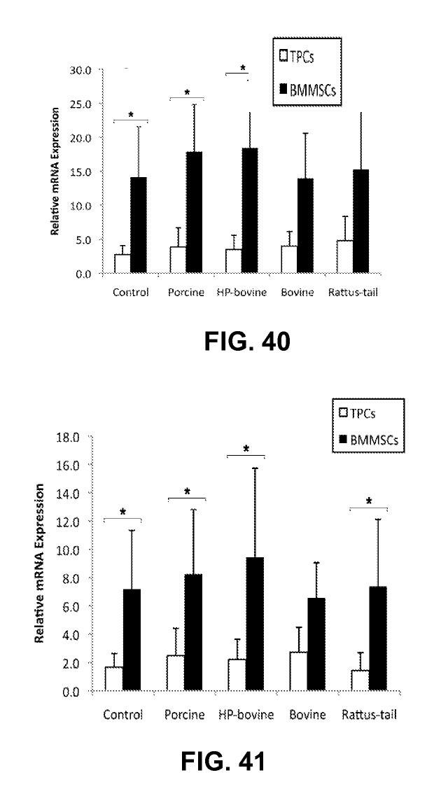

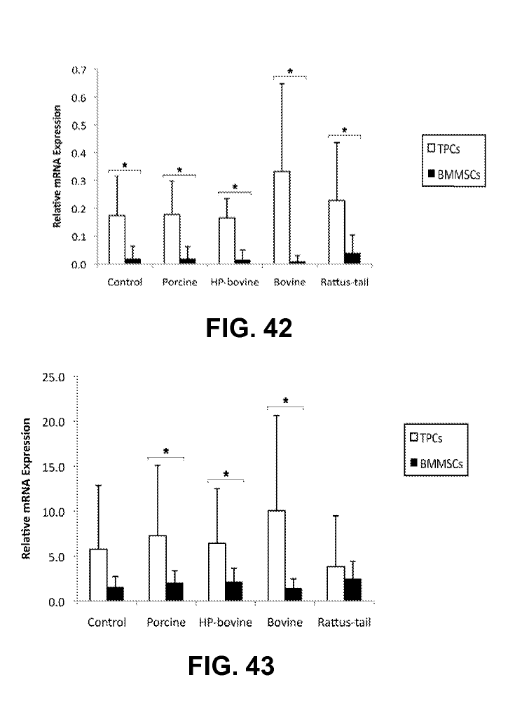

Gene expression: FIGS. 40-43 are graphs demonstrating the mean±standard deviation of the relative gene expression of collagen type I (FIG. 40), collagen type III (FIG. 41), COMP (FIG. 42), and decorin (FIG. 43) in BMMSCs and TPCs and cultured on each collagen group (control, porcine HP-bovine, and rattus (rat tail), determined at 7 days of culture. No differences in collagen type I, collagen type III, COMP, or decorin gene expression were observed between collagen groups and non-collagen controls for TPCs or BMMSCs (FIGS. 40-43). Relative to in vivo tendon gene expression, TPCs and BMMSCs expressed more collagen type I, collagen type III and decorin but less COMP. When comparing between cell types, BMMSCs expressed significantly more collagen type I when cultured on control, porcine and highly-purified collagen, and more collagen type III when cultured on control, porcine, highly-purified collagen, and rat collagen-coated surfaces. Tendon progenitor cells expressed significantly more COMP when cultured on control and all collagen groups, and decorin when cultured on porcine, highly purified bovine and bovine collagen.

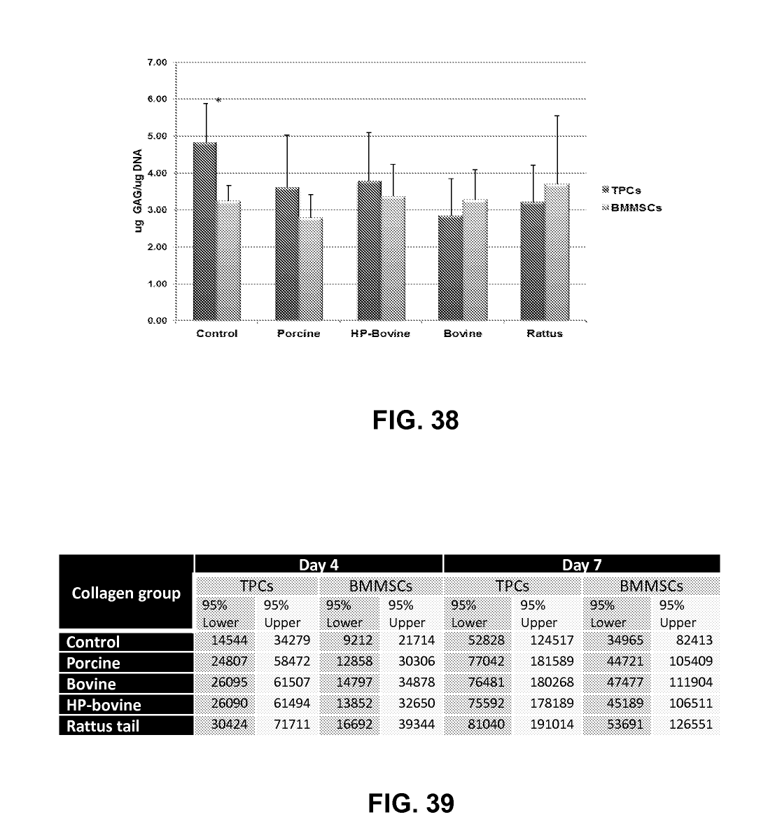

Glycosaminoglycan—FIG. 38 shows a graph demonstrating glycosaminoglycan (GAG) are the functional side chains of proteogylycans the concentration is a measurement of tendon structure and function. GAGs facilitate collagen fibril sliding and are critical extracellular matrix components. Decellularization resulted in GAG loss.

Scleraxis

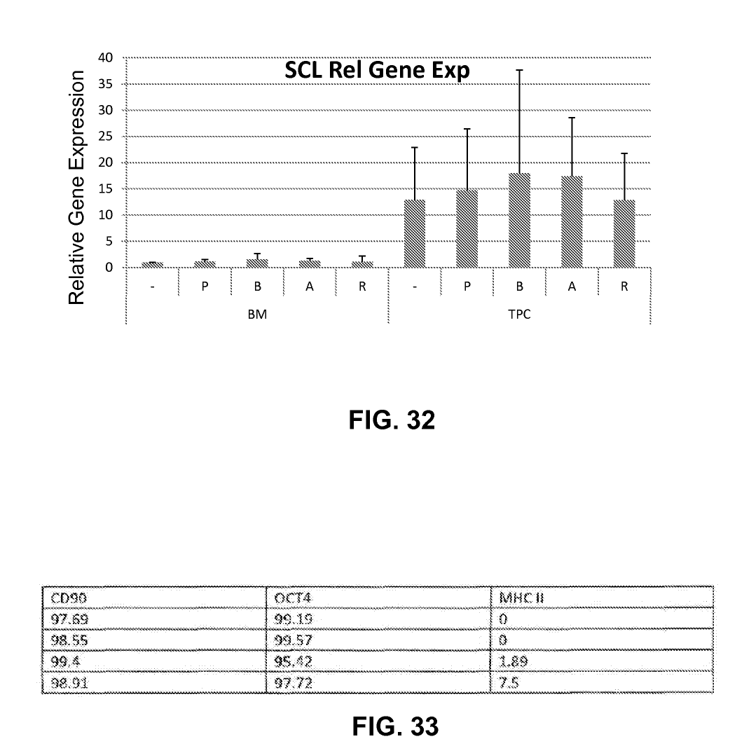

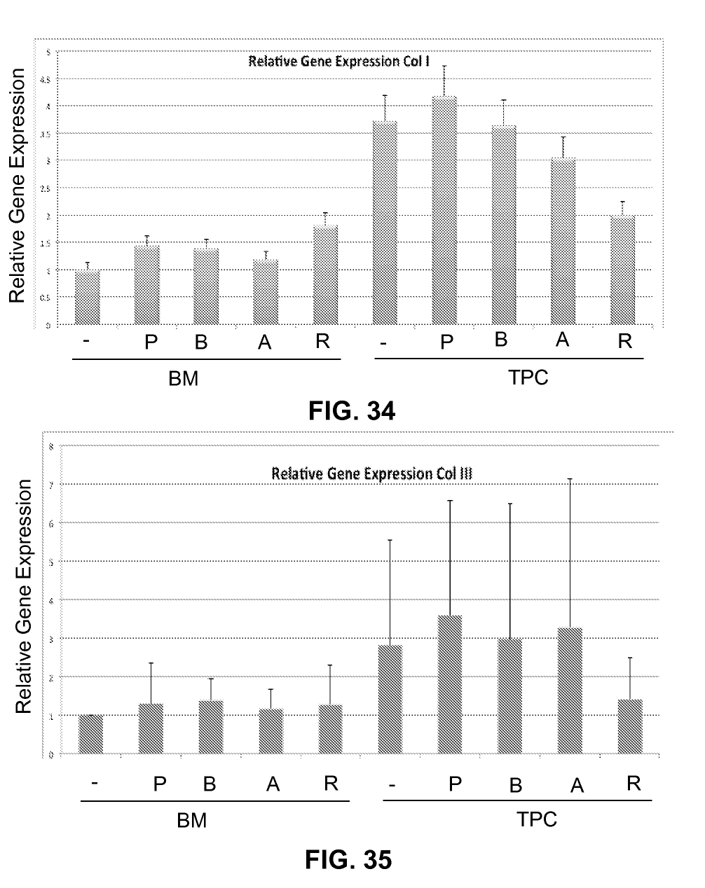

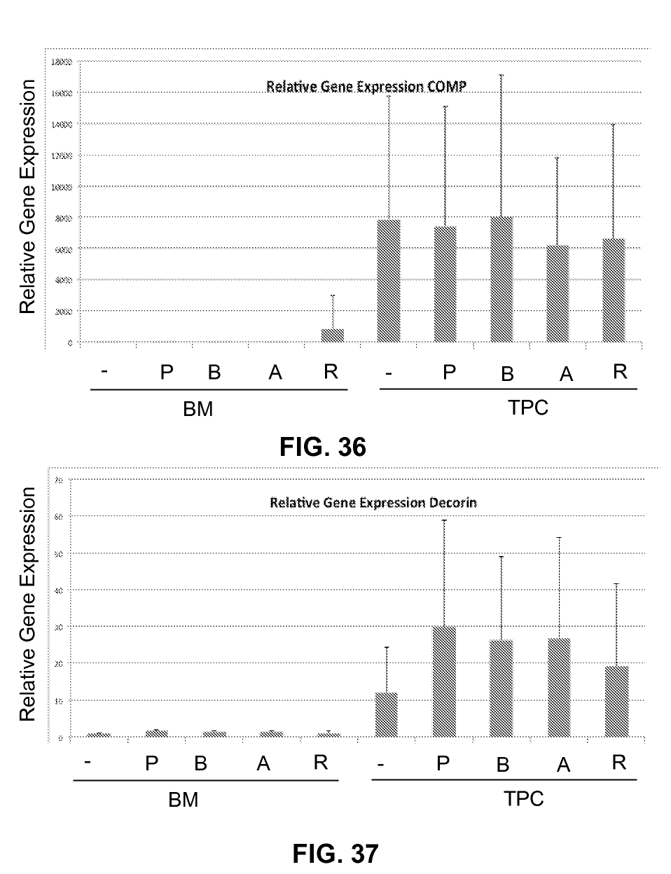

FIG. 32 shows a graph demonstrating scleraxis (SCL) relative gene expression (X-axis) in TPCs and bone marrow MSCs (BM). TPCs demonstrated a greater expression of SCL compared to BM. Scleraxis is a basic helix-loop-helix transcription factor that plays a central role in promoting fibroblast proliferation and matrix synthesis during the development of tendons.

Cell Surface Marker Expression: FIG. 33 shows a table containing flow cytometry data from TPCs where expression of CD90, OCT4, and MHC II was examined. TPCs were observed to exhibit markers which identify tendon cells such as high expression (greater than 90%) of CD90 and OCT 4, while simultaneously low in MHCII (less than 10%).

### Example 5

### Isolation of Tendon Cells

Tendon tissue (e.g. about a 2 cm×2 cm×6 cm piece) was dissected from the outer covering of the tendon (e.g. superficial digital flexor tendon) and kept in warm media until processing. The tendon tissue was weighed and placed in PBS supplemented with 1% Pen/Strep. The tendon tissue was minced into fine pieces using a scalpel blade and minced tissue was placed in a 50 mL conical tube and centrifuged at about 500×g for about 5 minutes. The formed pellet was washed twice by resuspending the pellet in about 15 ml of PBS supplemented with 1% Pen/Strep and re-centrifuging and repeating the PBS wash and centrifugation. After the final centrifugation, the pellet was resuspended in a collagenase containing about 0.2% collagenase (e.g. Worthington Collagenase II Cat. No. LS004177) in serum-poor (about 1% FBS) high-glucose DMEM with 1% Pen/Strep and L-glutamine (filter sterilized). About 10 mL collagenase containing solution was used per about 1 g of tendon tissue.

The resuspended pellet was incubated in the collagenase containing media overnight (not to exceed about 16 hours) at 37° C. with shaking (about 150 rpm). After digestion, the solution was centrifuged at about 500×g for about 10 minutes. The resulting pellet was resuspended in 15 mL of a solution containing 2% dispase (e.g Roche Dispase II, neutral protease grade II Cat. No. 0165-859) in serum-poor (about 1% FBS) high-glucose DMEM supplemented with 1% Pen/Strep and L-glutamine (filter sterilized). The resuspended pellet was incubated in this medium for about 1 hour at 37° C. with shaking (about 150 rpm). After shaking, the mixture was centrifuged at about 500×g for about 10 minutes.

The resulting pellet was resuspended in Tendon Medium: high-glucose (4.5g/dL) DMEM with glutamine; 1% v/v Pen/Strep; 10% v/v; 10% v/v CELLect Silver FBS; and 10% v/v horse serum. The resulting solution was filtered through a 100 micron mesh filter by gravity filtration. The filter was washed 3 times with media to collect cells. An optional cell count was performed. Collected cells were plated on tissue/cell culture plates (e.g. T75 culture flasks).

Plated cells were feed every two days after having had about 4 days to attach to the cell culture plate. When cells were about 80% confluent, cells were passaged.

Plates were assessed for homogeneous/spinoloid cell phenotype. Plates having spindle-shaped and a homogeneous monolayer of cells were trypsinized.

When cells were frozen, a freeze media (10% v/v DMSO, 25% v/v serum (CELLect Silver FBS) was used.

### Example 6

### Equine Bone Marrow MSCs

Bone marrow was aspirated aseptically from the tuber coxae via bone marrow biopsy needles into a 30 ml syringe containing about 1,000 units of heparin. Cells were centrifuged and resuspended in low-glucose DMEM supplemented with 1% Pen/Strep and glutamine, and 10% FCS. Cells were plated onto tissue culture plates (T75s). Bone marrow cells were cultured as described with tendon cells in Example 5 except a MSC monolayer media was used.

### Example 7

### A Bioreactor System for In Vitro Tendon differentiation and Tendon Tissue Engineering

Introduction

Tendon dysfunction occurs with high morbidity in both humans and animals, compromising freedom of movement and quality of life. Tendons are predominantly composed of hierarchically organized, aligned collagen fibrils, and the specialized structure of tendon extracellular matrix (ECM) provides tensile strength while transferring mechanical stimuli to resident cells.^{2,3} There is a reciprocal relationship between ECM properties and cellular behavior, and success of in vitro cultivation of tendon is dependent on recapitulating the natural environment of the tissue.

The horse is a model organism for studies of human tendon pathophysiology. Injury of the flexor digitorum superficialis tendon (FDST) is particularly common, and significant research has been dedicated to addressing the poor intrinsic regenerative capacity of this tissue. Mesenchymal stem cell (MSC) implantation has been safely used in the treatment of tendon degeneration, and there is some evidence that the multipotency and immunomodulatory properties of MSCs may improve healing. Seeding cells on scaffolds influences cellular behavior⁹ and supports endogenous repair, but the utility of current commercial tendon augmentation products remains limited. An equine decellularized tendon scaffold (DTS) has been developed in our laboratory as a step toward derived scaffolds have the benefit of (1) biochemical composition, (2) three-dimensional topography, and (3) tissue-relevant mechanical properties. DTS is suitable for MSC culture under static conditions, and it was hypothesized that subjecting these constructs to mechanical stimulation would induce differentiation toward tendon and produce viable regenerative graft materials. From previous bioreactor studies on tendon it is evident that, while loading is required for maintenance of a differentiated tenocytic phenotype and tissue biomechanical properties, mechanically stimulated tendon constructs exhibit sensitivity to the characteristics of mechanical stimuli. This cellular response is likely tissue- and model-dependent, requiring optimization based on construct properties and environmental conditions.

The aim of this experiment was to compare three deformation protocols on MSC-seeded DTS by examining the influence of strain on MSC phenotype. Two dynamic strain regimens of varying amplitude (3% and 5%) were selected based on their physiological relevance and compared to static (0%) controls. The approximate biomechanical transition between the toe region and the linear elastic region of deformation of the tendon stress—strain curve is 3% strain, while 5% is a standard linear amplitude of normal usage conditions. Despite the seemingly minor differences between these two groups, it was hypothesized that the distinctive biomaterial behaviors delineating the two deformation regions would differentially translate mechanical stimuli to resident cells. We hypothesized that both 3%- and 5%-strained constructs would exhibit stronger evidence of tendon differentiation than static culture. Furthermore, we anticipated the 3% strain protocol would effectively induce tendon differentiation in adult MSCs and promote an anabolic response. Effects of the three strain protocols were evaluated via the expression of tendon marker genes, biomechanical properties, and production of ECM following 11 days of bioreactor culture.

Materials and Methods

Experimental Design: MSC-seeded DTS was divided into three groups by strain amplitude: referenced in the text as the 0%, 3%, and 5% experimental groups. Microscopy, composition, and biomechanics data references either initial DTS (iDTS), control DTS (cDTS), or both as negative controls. iDTS is the freshly prepared scaffold material, subject to no further manipulation. cDTS was not seeded with cells, but underwent identical incubation conditions to the 0% experimental group. Adult tendon (FDST) was used as a control to compare bioreactor gene expression data with mature whole tissue (n=4).

Production of Decellularized Tendon Scaffolds (DTS): DTS was produced using sterile technique in accordance with methods developed in our laboratory.12 FDSTs were surgically excised from the forelimbs of four adult sport horses aged 4.5-1.7 years, euthanized as a result of unrelated conditions in accordance with the Institutional Animal Care and Use Committee of Virginia Tech. Tendons from these horses were longitudinally sectioned using an electric dermatome (Integra Lifesciences, Plainsboro, N.J.) into ribbons 400 mm in thickness, then divided into samples 10mm-45mm in surface area. Briefly, these samples were decellularized by four freeze-thaw cycles, a 48-h detergent infusion with 2% SDS (Sigma, St. Louis, Mo.) in 1M Tris-HCl, pH 7.8 (Fisher Scientific, Waltham, Mass.) at 4° C., incubations in 0.05% trypsin-EDTA (Gibco, Carlsbad, Calif.), 10 mg/ml DNase-I (Stemcell Technologies, Vancouver, Canada) and 95% ethanol (Sigma), followed by repeated washings in H2O in a gyratory shaker (New Brunswick Scientific, Edison, N.J.). The resulting scaffolds were frozen at −20° C. prior to use. Reference FDST samples for RNA analysis were flash frozen using liquid nitrogen and stored at −80° C. prior to processing in the same manner as the DTS samples (described below).

Derivation of Primary Mesenchymal Stem Cell (MSC) Lines: MSCs were collected and assessed via routine processing techniques (Stewart A A, Barrett J G, Byron C R, et al. 2009. Comparison of equine tendon-, muscle-, and bone marrow-derived cells cultured on tendon matrix. Am J Vet Res 70:750-757) using bone marrow aspirate collected from the sternum of the same four donor horses as the DTS material. Cells were cultured at 37° C., 5% CO2, and 90% humidity in standard MSC media: low-glucose GlutaMAX DMEM with 110 mg/ml sodium pyruvate (Gibco) plus 10% MSC FBS (Gibco) and 100 U/ml sodium penicillin, 100 mg/ml streptomycin sulfate (Sigma). Cells were expanded in monolayer culture to 80% confluence and passaged twice. Flow cytometry was conducted on these four separate cell lines with a BD FACSCalibur using monoclonal antibodies previously validated in our laboratory (data not shown). The results demonstrated that approximately 90% of cells were positive for CD-90 and CD-44, common MSC surface antigens, as well Oct-4, which is indicative of a naive sternness found in both embryonic and adult stem cell populations.

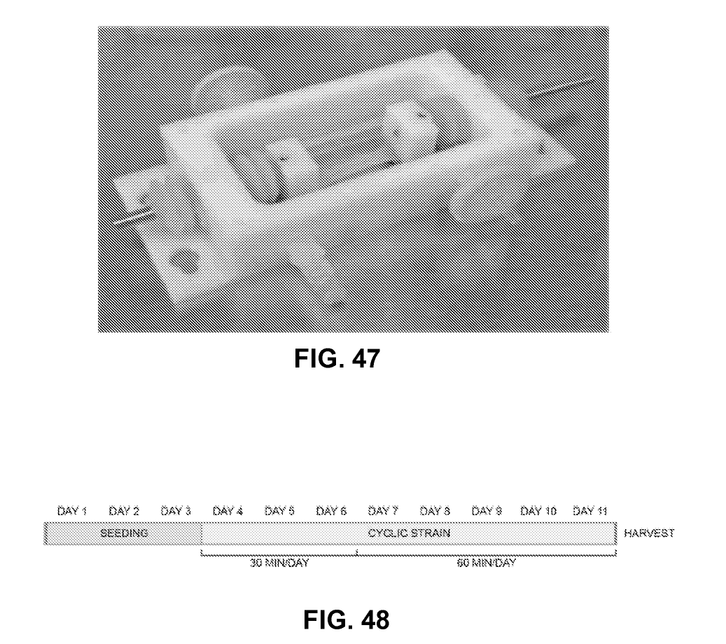

Construct Seeding and Bioreactor Culture: Following MSC expansion, DTS was thawed and saturated in tendon cell culture media: standard MSC media as previously described with the addition of 35.7 |ig/ml L-ascorbic acid (Sigma). This medium was used for the remainder of the study. DTS samples were clamped into the bioreactor vessels along their natural axis of alignment (FIG. 47), obscuring 0.5 cm on each end. MSC suspensions were deposited via micropipette over syngeneic DTS at a density of 20,000 cells/cm², which equates to the approximate surface density of a 40% confluent monolayer and had previously been validated.¹² Seeded DTS was subsequently placed in an incubator for 72 h to allow cells to adhere, with the vessels filled to their maximum media volume of 6 ml after the first 24 h. Following this seeding period, bioreactor culture was initiated, and half of the media was changed every 2-3 days.

A custom bioreactor at the Tissue Engineering Resource Center was used in this study. The device, described previously (Matheson L A, Jack Fairbank N, Maksym G N, et al. 2006. Characterization of the Flexcell™ Uniflex™ cyclic strain culture system with U937 macrophage-like cells. Biomaterials 27:226-233), incorporates self-contained tissue culture vessels that allow samples to be individually clamped and mechanically manipulated. A LabVIEW program (National Instruments, Austin, Tex.) operates four stepper motors in parallel stages. MSC-seeded DTS constructs were cultured in this bioreactor for a total of 11 days: three days without stimulation, 3 days subject to displacement for 30min per day, then 5 days at 60min per day (16. Whitlock P W, Seyler T M, Northam C N, et al. 2013. Effect of cyclic strain on tensile properties of a naturally derived, decellularized tendon scaffold seeded with allogeneic teno-cytes and associated messenger RNA expression. J Surg Orthop Adv 22:224-232) (FIG. 48). Samples underwent linear deformation at 0.33 Hz according to their experimental group. These parameters were selected due to their physiological relevance as well as reports that as few as 5-7 days of bioreactor culture at 0.0167-0.5 Hz are sufficient to observe improvements in material properties in fibroblast-laden tendon/ligament constructs. All groups were repeated in triplicate for a total of 36 vessels: four horses, three experimental groups, and three replicates. After the final day, constructs were removed from their vessels, divided for assays and either flash frozen in liquid nitrogen or immersed in a fixative for preservation prior to analysis.

RNA Isolation and Gene Expression Analysis

Half of each sample was used for gene expression analysis, and all samples within each treatment group were pooled. The experiment was repeated in its entirety to produce a replicates. Bioreactor constructs were transferred from storage at −80° C. directly into a cryomill (SPEX SamplePrep, Metuchen, N.J.) and pulverized in liquid nitrogen. RNA was isolated from tissue homogenates using an acid guanidinium thiocyanate extraction protocol in phenol-chloroform, followed by precipitation in isopropanol. The resulting pellets were resuspended and the solutions concentrated in RNeasy spin columns (Qiagen, Valencia, Calif.), quantitated with Ribo-Green RNA reagent (Life Technologies, Grand Island, N.Y.) and reverse-transcribed with a high-capacity cDNA kit (Life Technologies). cDNA was pre-amplified using a validated commercial TaqMan kit (Life Technologies) prior to reaction in a 7500 Real-Time PCR System (Applied Biosystems, Carlsbad, Calif.) using custom TaqMan probes (Life Technologies) in duplicate. A list of primers, probes, and abbreviations used are included in Table 3. Table 3 shows Custom-Designed Equine qPCR Primers and Probes Were Designed to Target: Glyceraldehyde 3-Phosphate Dehydrogenase (GAPDH), Scleraxis (SCX), Type-I Collagen (COL-I), Type-III Collagen (COL-III), Decorin (DCN), and Biglycan (BGN). Reactions were quantified with the 2-AACt method using GAPDH as a reference gene and are reported by fold-change with respect FDST.

TABLE 3

| Gene | Forward | Reverse | Probe |
|---|---|---|---|
| GAPD | CAAGTTCCA | GGCCTTTCCG | CCGAGCAC |
| | TGGCACAGT | TTGATGAC | GGGAAG |
| SCX | CGCCCAGCC | TTGCTCAACT | TCTGCACC |
| | CAAACAG | TTCTCTGGT | TTCTGCC |
| COL-I | GCCAAGAAG | TGAGGCCGTC | ACATCCCA |
| | AAGGCCAA | CTGTATGC | GCAGTCA |
| COL- | CTGCTTCAT | ATCCGCATAG | AACAGGAA |
| | CCCACTCTT | GACTGACC | GTTGCTG |
| DCN | AAGTTGATG | GGCCAGAGAG | ATTTGGCT |
| | CAGCTAGCC | CCATTGTC | AAATTGG |
| BGN | TGGACCTGC | AGAGATGCTG | TCTGAGCT |
| | AGAACAATG | GAGGCCTT | CCGAAAG |

Mechanical Testing: A representative sample was collected from one replicate of each FDST and experimental group to undergo failure testing (average dimensions 12.4±1.1 mm×1.7±0.1 ram). Following measurement with a digital caliper, samples were elongated at 0.5% per second until failure using a custom materials testing device controlled by National Instruments components, generating stress-strain curves. Elastic modulus was computed as the slope of the total linear region of this relationship. Ultimate tensile strength was calculated as the maximum force per unit area endured prior to failure.

Spectrophotometric Biochemistry Assays: Sulfated glycosaminoglycan (GAG) content was assayed using the 1,9-dimethylmethylene blue (Sigma) technique, referencing chondroitin sulfate A (Sigma). This procedure was conducted in aliquots obtained during media changes, as well as in each bioreactor construct on Day 11. Solid samples were solubilized by enzymatic digestion in papain (Sigma). cDTS was also included in this analysis, in addition to the typical FDST and iDTS controls, to isolate the influence of cells on GAG maintenance over time under experimental conditions and in tendon cell culture media. DNA content was quantified in the same digest solutions using a Quant-iT PicoGreen (Molecular Probes, Carlsbad, Calif.) assay to determine relative cell number. Solubilized collagen was measured using a Sircol kit (Biocolor Ltd., Carrickfergus, UK) in acid/salt-washed pepsin (Sigma) digests of solid samples following the conclusion of the experiment. Values are reported with respect to dry weight, obtained by dehydration in an oven.

Histology: A portion of each experimental sample, as well as of each FDST and iDTS control, was fixed in a freshly prepared solution of 4% paraformaldehyde (Sigma) and submitted for commercial histological preparation (Histoserv, Inc., German-town, Md.). Samples were embedded in paraffin, longitudinally sectioned into 5 urn slices, and stained with hematoxylin and eosin. Images were acquired using an Olympus IM inverted microscope and a Moticam 10 CMOS camera.

Statistical Analysis: Data are reported as mean±standard error in aE figures. One-way multivariate analyses of variance (MANOVA) with a repeated measures designs followed by standard F-tests were used to determine statistical significance of all data points except qPCR data (p<0.05). A standard one-way MANOVA was used for qPCR results. Results are annotated in figures alphabetically. One-way Student's i-tests were also used in biochemical and biomechanical analysis to specifically test experimental groups to DTS controls. Points of significance (p<0.05) are demarcated with an asterisk in applicable figures. Computation was performed in JMP Pro 11 (SAS Institute Inc., Rockville, Md.), Prism (GraphPad Software, Inc., La Jolla, Calif.) and Excel 14 (Microsoft, Redmond, Wash.).

Results

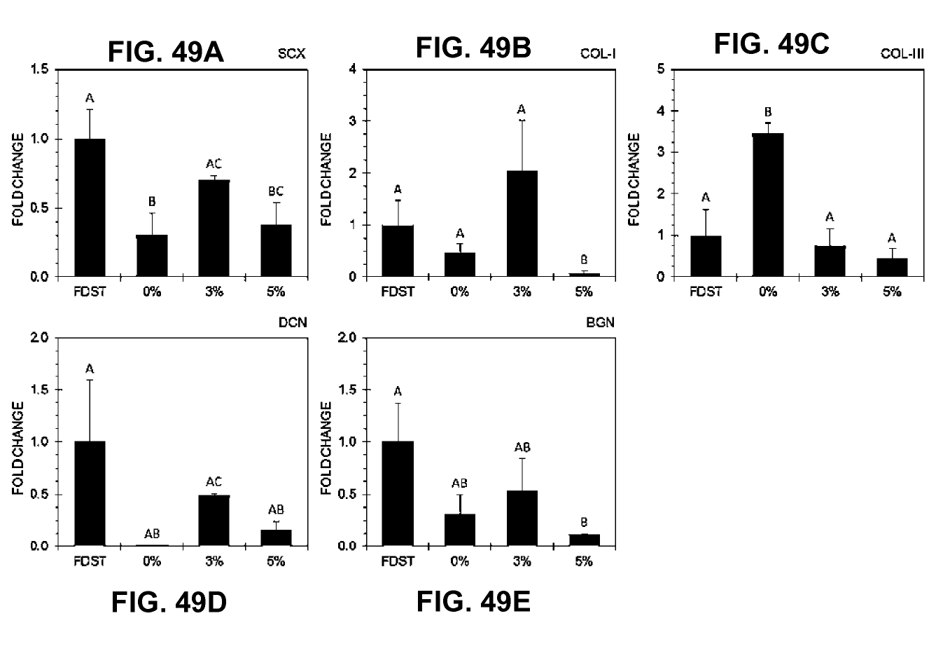

Cyclic Strain Promotes a Tenocytic Gene Expression Profile: Gene expression data are shown in FIGS. 49A-49E. SCX expression more than doubled from the 0% to the 3% experimental group to 70±15% of FDST, and this difference was significant (p=0.024). SCX in the 5% experimental group fell between the 0% and 3% groups, and was not statistically different from either group. COL-I expression was greatest in the 3% experimental group, with message levels present at 2.09±0.96 times what is observed in FDST: statistically greater than the 5% group (p=0.041). COL-III expression was dramatically upregulated in the 0% experimental group versus FDST (p=0.005). The ratio of relative COL-I to COL-III expression was 1.75 in the 3% experimental group, whereas it was less than or equal to 0.14 in the 0% and 5% experimental groups. DCN expression changed in response to bioreactor protocol, with greatest DCN expression in the 3% experimental group, significantly higher than in the 0% (p<0.001) or 5% (p=0.011) experimental groups. BGN was most expressed in the 3% experimental group, but this difference was not statistically significant.

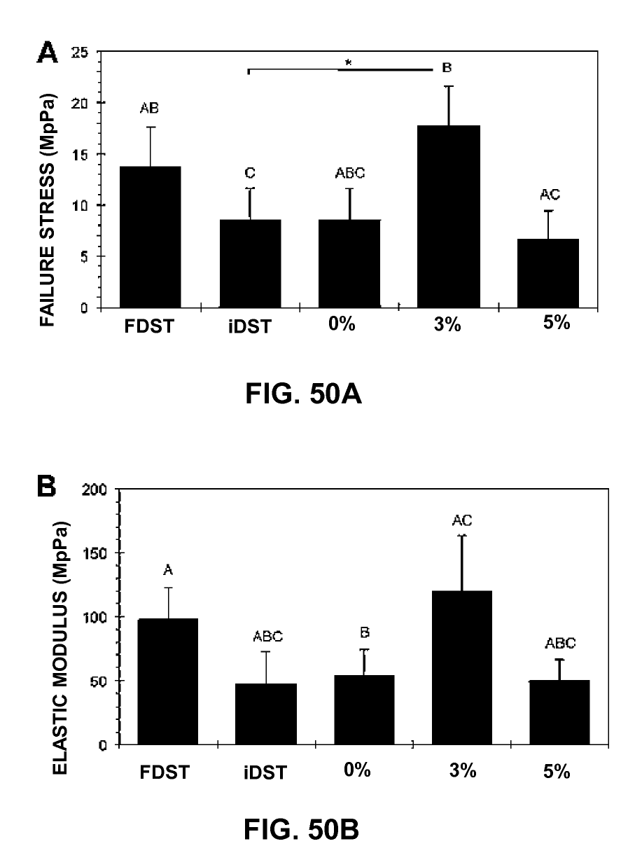

Three Percent Strained Constructs Mimic the Mechanical Properties of Native FDST: Constructs in the 3% experimental group failed at a mean stress of 17.7±3.8 MPa, which is more than double that of iDTS (p=0.041) (FIG. 50A). Constructs in the 0% and 5% experimental groups failed at significantly lower stresses than those in the 3% experimental group (p=0.009 and p=0.043, respectively). Constructs in the 0% and 5% experimental groups had significantly lower elastic moduli than FDST (p=0.034 and p=0.019, respectively), while this difference only approached significance for iDTS (p=0.090) (FIG. 50B). Relative to iDTS, the 3% experimental group exhibited a 2.56-fold increase in elastic modulus to 119±44MPa a value within 25% of the elastic modulus of matched native tendons (98±25MPa), and without statistical significance between the two.

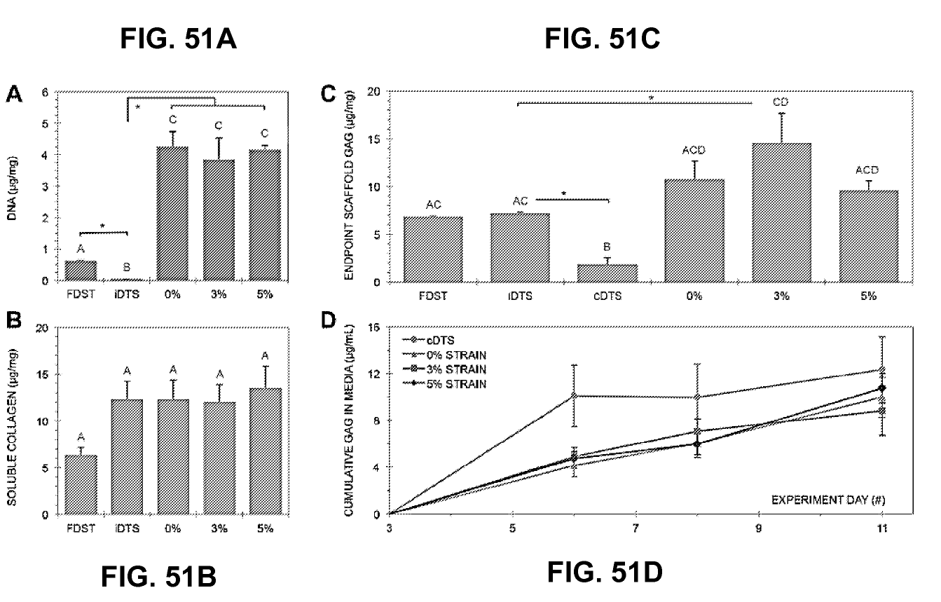

MSCs Integrate Into DTS and Modulate Scaffold Composition: Decellularization eliminated 95% of DNA from FDST, to 0.03 u,g/mg in DTS (p<0.001). All MSC-seeded bioreactor constructs had significantly more DNA than native tendon (p<0.001), equating to 6.6±0.2 times the value of FDST (FIG. 51A). There were no statistical differences in DNA content between the 0%, 3%, and 5% experimental groups. Soluble collagen production from the 3% experimental group after 11 days was 12.0±1.9 (xg/mg (FIG. 51B). There were no significant differences in soluble collagen between groups.

Endpoint GAG composition in the 3% experimental group increased by a factor of 2.14 relative to iDTS to 13.5±3.1 |xg/mg (p=0.050), while unseeded cDTS released 74% of GAG content into the culture media (p=0.004) (FIG. 51C). GAG release into culture media was tracked cumulatively, and it was found that cDTS lost 10.1±2.6 u.g/ml of GAG in the first three days (FIG. 51D). In contrast, the mean GAG release of the 0%, 3%, and 5% experimental groups was 4.6 ±0.26 (xg/ml 3 days into the bioreactor culture period. There was no further GAG release between Days 8 and 11 in the 3% experimental group (p=0.106).

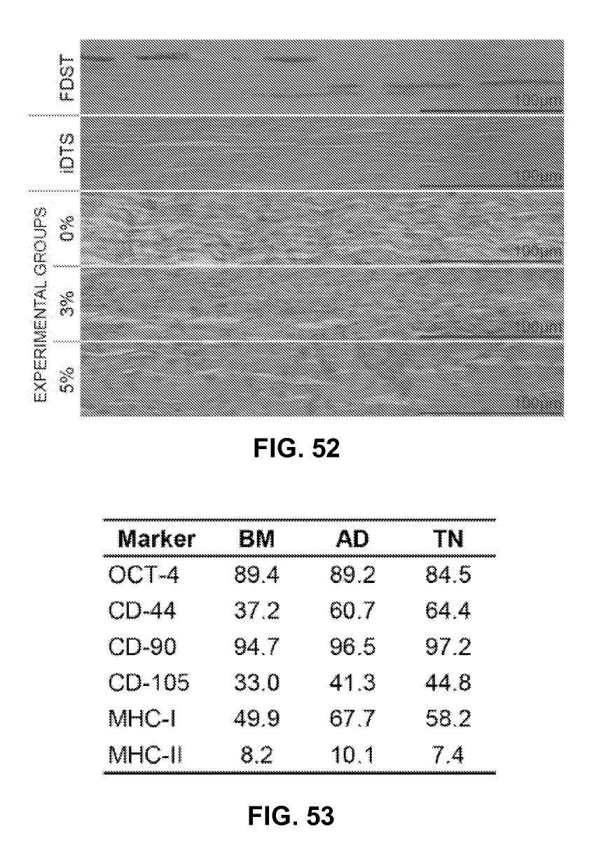

Histological examination confirmed the high cellularity of MSC-seeded constructs relative to native FDST (FIG. 52). Cells integrated at least 200 u.m. deep into DTS by 11 days. Cells also established an anisotropic phenotype, elongating parallel to the DTS collagen fibers.

### Example 8

### Tenogenesis of Bone Marrow-, Adipose-, and Tendon-Derived Stem Cells in a Dynamic Bioreactor

Introduction

Tendons connect muscles to bones, and act as springs to store and transmit force to the skeletal system during locomotion. Tendon injury leads to decreased quality of life due to pain and loss of function. Tendons are hypocellular tissues composed of aligned, hierarchically organized extracellular matrix (ECM): predominantly fibrillar collagens. The biomechanics of tendon improve efficiency of locomotion, and forces on the tendon are translated to the cellular level where they provide important mechanobiological signals to resident tendon cells. Tendinopathies are widely believed to result from the progressive buildup of microstructural damage during overuse, leading to abnormal biomechanical signals to the cells resulting in altered cellular phenotypes and ECM composition. The therapeutic application of adult mesenchymal stem cells (MSCs) of autologous or allogeneic origin may help regenerate acute or chronic tendon damage not only by promoting tissue neogenesis, but also by modulating inflammation ]and providing trophic support.

BM (bone marrow) and AD (adipose) are established MSC sources often studied in regenerative medicine applications. While they share major phenotypic similarities, transcriptional and proteomic differences suggest preferential pre-commitment to certain mesenchymal lineages. Thus, tissue-specific stem cells may have better regenerative efficacy in their tissue of origin. TN (tendon)-derived MSCs (also known as tendon stem/progenitor cells) possess similar mesenchymal-lineage mulitipotency to BM or AD MSCs, respond to exercise, and have unique features based on their specific origin. Stem cells enhance healing of damaged tendon, but a lack of standardization in pre-transplantation processing techniques complicates controlled evaluation of relative efficacy across studies. Better in vitro models are needed to characterize the use of these cells.

Tissue-engineered tendon graft material can be manufactured using any of these cell types, but proper selection of cell source and scaffold origin are important decisions in optimizing graft design. The discovery that tendon tissue contains populations of cells with characteristics of both MSCs and tenocytes has prompted investigation of their use in animal models of tendon regeneration, with mixed results. TN MSCs have however demonstrated improved collagen alignment, stronger graft mechanical strength and a decreased tendency for ectopic ossification versus BM cells when used to augment surgical repair of full-sized defects in a rat model. TN cells also promote tenogenesis of allogenic MSCs via paracrine signaling or cell-cell contact, which may enhance extrinsic tendon healing in vivo. Further investigation into the tissue-regenerative properties of TN MSCs is required to evaluate their potential use in cell therapy.

Biomimetic tissue culture systems enhance experimental control and decrease the number of animals used in pre-clinical investigations. Bioreactors to study tendon and ligament cell behavior have been around for over a decade, and it is now evident that mechanical stimulation dramatically enhances tenogenic differentiation of MSCs and can be used to precondition graft materials. Isolated components of natural extracellular matrix provide important cues for in vitro cell culture. Moreover, intact decellularized scaffolds provide near-native mechanical properties and provide further opportunities to assess tissue remodeling ex vivo. A number of studies have demonstrated differentiation and cell-mediated improvements in tissue mechanical properties in response to tendon-like ultrastructure and strain. However, at present there are only three other groups working with decellularized tendon scaffolds in bioreactors, and there are no published experiments comparing stem cell differentiation in these systems. The aim of this study was to determine to what degree the tendon differentiation and ECM anabolic responses of MSCs are dependent on their source of origin, and discover whether the use of particular cell types is advantageous for tendon graft maturation or cell therapy.

In order to evaluate the relative regenerative potential of BM, AD and TN MSCs, this study applied a bioreactor protocol previously used to evaluate amplitude-dependent behavior of MSCs in response to cyclic strain. The bioreactor is designed to simulate gentle exercise, while decellularized tendon scaffolds (DTS) provide “biophysical beacons” such as native topography and force translation to cells. Outcomes were assessed using microscopy, qPCR, biochemical analysis and tensile testing. It was hypothesized that TN MSCs would integrate into DTS, exhibit a more tenocytic gene expression profile compared to BM and AD MSCs and improve tissue mechanical properties.

Materials and Methods

Experimental design: Matched DTS and MSC cell lines were obtained from four adult sport horses aged 4.75±1.75 years, euthanized with Institutional Animal Care and Use Committee approval. Tissues and cell lines were cryopreserved until ready for use in a −80° C. chest freezer or in liquid nitrogen, respectively. Donor tissues for cell lines include sternal bone marrow (BM), subcutaneous adipose (AD) and flexor digitorum superficialis tendon (FDST) (TN). Matched FDST tissue and DTS are included as control groups, with the exception of qPCR data, which references a bank of four unrelated adult sport horses, as suitable syngeneic samples were unavailable.

Evaluation of Regenerative Potential of Bone Marrow, Adipose, and Tendon derived stem cells: Flow cytometry was used to evaluate MSC cell surface markers in stem cells derived from bone marrow (BM), adipose tissue (AD), and tendons (TN). Results are demonstrated in FIG. 53.

Decellularized tendon scaffolds: DTS samples measuring 45 mm×10 mm×400 μm were produced from forelimb FDST obtained at necropsy. The decellularization process has been described in detail elsewhere [46]. Briefly, longitudinally-sliced tendon sections underwent four freeze-thaw cycles, 48 hours of detergent decellularization in 2% SDS (Sigma), enzymatic cleanup with 0.05% trypsin-EDTA (Life Technologies) and 10 μg/mL DNase-I (STEMCELL Technologies), and washing steps with 95% ethanol (Sigma), H₂O and PBS (Lonza). Residual SDS was detected at 71.7±30.7 ng/mg, orders of magnitude below cytotoxic levels (data not shown). DTS was frozen until ready for use, at which point samples were thawed and saturated with tissue culture media.

Cell isolation and culture: MSCs were derived from primary tissue samples using routine isolation protocols reliant on cell separation techniques and adherence to tissue culture plastic. All cell culture was conducted at 37° C., 5% CO₂ and 90% humidity, with manipulations performed in a BSL2 biosafety cabinet (NuAire), including 50% media changes every 2-3 days. The following media cocktails were used for monolayer expansion through two passages—BM MSCs: low-glucose GlutaMAX DMEM with 110 μg/mL sodium pyruvate (Gibco), 10% MSC FBS (Sigma) and 100 U/mL sodium penicillin, 100μg/mL streptomycin sulfate (Sigma), AD and TN MSCs: high-glucose GlutaMAX DMEM with 110 μg/mL sodium pyruvate (Gibco), 10% Cellect Silver FBS (MP Biomedicals), 10% Horse serum (Life Technologies) and 100 U/mL sodium penicillin, 100 μg/mL streptomycin sulfate (Sigma). Colony forming unit (CFU) assays were performed for each cell line at P2 by plating 1,000 cells on plastic 100 mm-diameter cell culture dishes (Thermo Scientific) in triplicate. After 9 days, cells were fixed in 4% paraformaldehyde (Sigma) and refrigerated overnight. Colonies were then washed and stained with 0.05% crystal violet (Fisher Scientific) and photographically counted in ImageJ (National Institutes of Health).

Cell-laden bioreactor constructs: BM, AD and TN MSCs were seeded in suspension directly over the surface of DTS at 250,000 cells per construct in a two-stage 333 μL solution transfer procedure separated by 20 minutes. Samples were paired with their technical replicates in 60 mm petri dishes (Thermo Scientific) and incubated for 24 hours to facilitate MSC adhesion to DTS. All bioreactor constructs regardless of cell type were cultured in TN MSC media as previously described, with the exclusion of streptomycin [47] and the addition of 35.7 μg/mL L-ascorbic acid (Sigma). After seeding, individual samples were carefully clamped into custom-fabricated bioreactor vessels, in which they remained for the following 10 days with regular media changes.

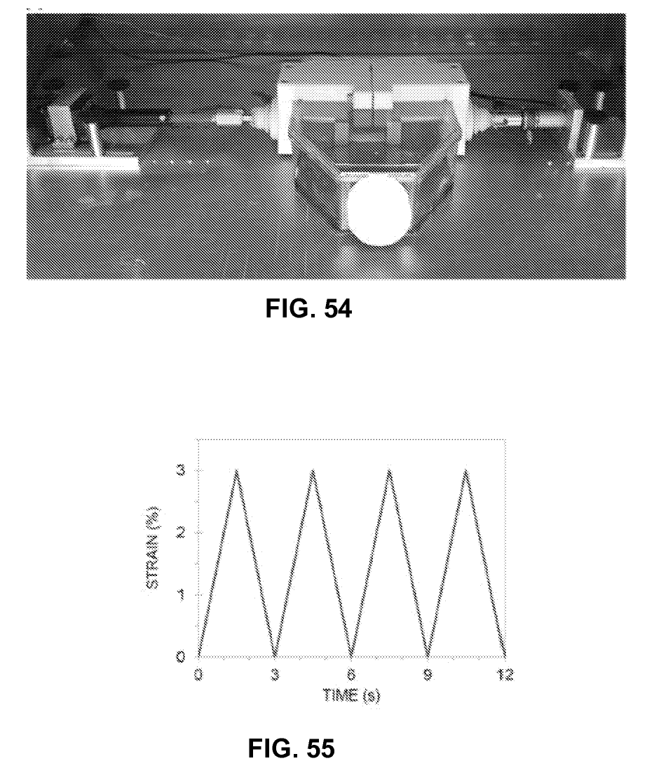

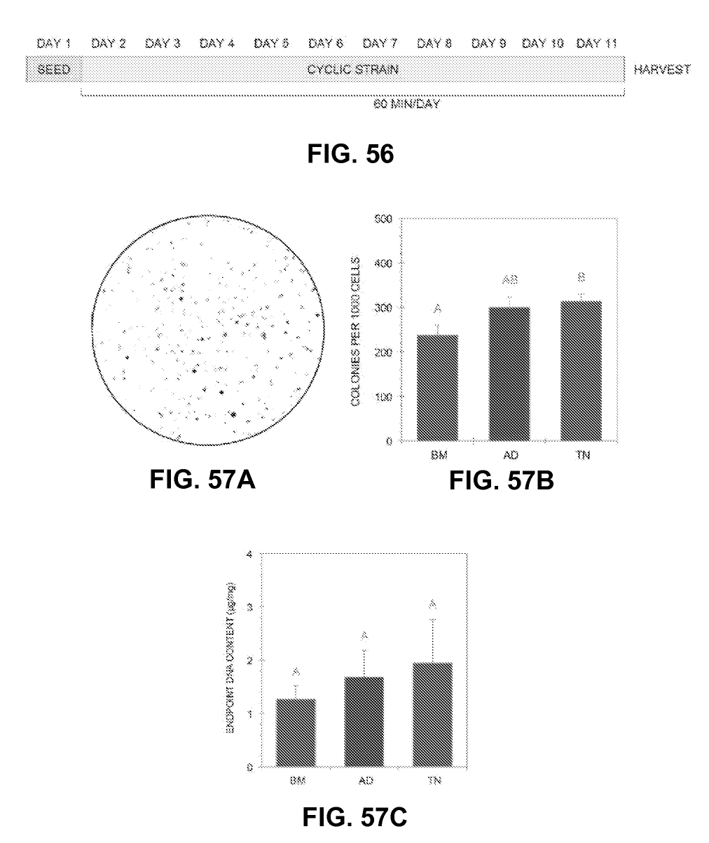

Cyclic strain bioreactor: This Example implemented a custom bioreactor FIG. 54 (See also e.g. FIGS. 1-12). Aluminum stages anchor three opposed pairs of load cells (Honeywell, Model 31) and NEMA 11 captive linear actuators (Hayden-Kerk) driven by microstepping chopper drives (Hayden-Kerk). Aluminum clamps stabilized by polytetrafluoroethylene brackets were built around T-175 tissue culture flasks. This hardware is run by National Instruments modular units, including a CompactRIO 9076 controller and chassis, a NI 9237 analog input module, and three NI 9512 stepper drive interfaces. Custom software for bioreactor culture and tensile testing was designed in LabVIEW. Cell-laden bioreactor constructs were subject to one hour of daily cyclic stretching: 3% strain at 0.33 Hz (FIG. 55). After 10 days, samples were cut from their vessels and divided for assay (FIG. 56). Quantitation of gene expression: To produce qPCR samples for each tissue type and cell line, 70% of each technical duplicate construct was pooled. After harvest, these samples were snap-frozen in liquid nitrogen and immediately ground in a cryomill (SPEX SamplePrep). The resulting homogenates were suspended in guanidinium thiocyanate lysis buffer before undergoing phenol-chloroform separation and an overnight isopropanol precipitation (Life Technologies and Sigma). The pellets were then purified using RNeasy spin columns (Qiagen) and quantified using a NanoDrop spectrophotometer (Thermo

Scientific) before reverse-transcription with a commercial cDNA kit (Life Technologies). Duplicate single-plex reactions were conducted in an Applied Biosystems 7500 Real-Time PCR System using custom TaqMan (Life Technologies) primers and probes on a list of gene targets outlined in Table 4, abbreviated as follows: scleraxis (SCX), tenomodulin (TNMD), collagen type-I (COL-I), collagen type-III (COL-III), decorin (DCN), biglycan (BGN), elastin (ELN), cartilage oligomeric matrix protein (COMP), and major histocompatibility complex classes I (MHC-I) and I (MHC-II). Reactions were quantified using the 2^{-ΔΔCt} method using glyceraldehyde 3-phosphate dehydrogenase (GAPDH) as a previously validated housekeeping gene.

TABLE 4

| | FORWARD | REVERSE | |
|---|---|---|---|
| TARGET | PRIMER | PRIMER | PROBE |
| GAPDH | CAAGTTCCATG | GGCCTTTCCGTT | CCGAGCACGG |
| | GCACAGTCAAG | GATGACAA | GAAG |
| SCX | CGCCCAGCCCA | TTGCTCAACTTT | TCTGCACCTT |
| | AACAG | CTCTGGTTGCT | CTGCC |
| TNMD | GCCGCGTCTGT | CGCCCTCCTTGG | CTAGGTTACT |
| | GAACCTTT | TAGCAGTA | ACCCGTATCC |
| COL-I | GCCAAGAAGAA | TGAGGCCGTCCT | ACATCCCAGC |
| | GGCCAAGAA | GTATGC | AGTCACCT |
| COL-III | CTGCTTCATCC | ATCCGCATAGGA | AACAGGAAGT |
| | CACTCTTAT | CTGACCA | TGCTGAAGG |
| DCN | AAGTTGATGCA | GGCCAGAGAGCC | ATTTGGCTAA |
| | GCTAGCCTG | ATTGTCA | ATTGGGACTG |
| BGN | TGGACCTGCAG | AGAGATGCTGGA | TCTGAGCTCC |
| | AACAATGAGAT | GGCCTTTG | GAAAGG |
| ELN | TGGCTGCGGTC | TGTGACCGAATC | TACCCAAGCA |
| | CAGTTG | CAGCTTGA | TCGAAAG |
| COMP | GAGATCGTGCA | GACCGTATTCAC | CTGGCTGTGG |
| | AACAATGAA | GTGGAAC | GTTACA |
| MHC-I | GGAAGCGCTCA | GGCACTGTCACT | AGGAGGGAGC |
| | GGTGGAA | GCTTGCA | TACGC |
| MHC-II | AACCCTCACCC | GGTGGTCAGCCA | TGAGCACCTG |
| | TGATCACCAT | CAATGTCTT | TGGAGGT |

Microscopic Imaging: Histological samples were placed in solutions of 4% paraformaldehyde (Sigma) and commercially embedded in paraffin, sectioned into 5 μm-thick slices, and stained with hematoxylin and eosin (Histoserv, Inc.). Histology images were taken using an Olympus IM inverted microscope and a Moticam 10 CMOS camera. Representative samples for confocal imaging were collected at the completion of the experiment, labeled with calcein-AM and DAPI, and freshly mounted for fluorescence microscopy. Confocal images were acquired at the Microscopy Suite at James Madison University using a Nikon Eclipse TE2000 laser scanning confocal microscope. Confocal images represent z-stacks approximately 100 μm in thickness. Additional images were acquired using an AMG EVOS_{FL} digital inverted fluorescence microscope after probing representative samples with a live/dead kit (Invitrogen).

Biochemical composition: A quantitative assay for sulfated glycosaminoglycan content was conducted on all media aliquots and on samples of all constructs, using 1,9-dimethylmethylene blue (DMMB) referencing bovine chondroitin sulfate A (Sigma). Solid samples for DMMB analysis were first dehydrated and digested in papain. A second set of samples from all constructs was digested in pepsin (Sigma) and analyzed for soluble collagen content using a Sircol kit (Bicolor). DNA content was also quantified using a NanoDrop spectrophotometer on pepsin digests.

Mechanical testing: Daily secant moduli were calculated from the maximum stress/strain values between cycles 40-60 during each bioreactor session. After the final day, tensile testing was conducted on portions of samples from each horse and cell line (length: 17.26±2.73 mm, width: 2.48±0.33 mm), submerged in PBS. These samples underwent 11 priming cycles of 3% strain at 0.33 Hz immediately followed by an extension of 0.1% strain per second to failure. Elastic modulus was computed as the slope between two points in the linear deformation region of the stress/strain curve, and failure stress was calculated as the maximum load per cross-sectional area.

Statistical analysis: All data is represented as mean ±standard error. Statistical significance was determined via one-way multivariate analyses of variance using standard F-tests. A repeated measures design was used for all analyses except for qPCR comparisons with FDST, as these samples were not syngeneically matched as all other sets were. Significance (p≦0.05) is presented alphabetically in bar graphs and symbolically with asterisks in line graphs. Computations were performed in JMP 11 (SAS Institute, Inc.) and figures were designed in Excel 14 (Microsoft).

Results

BM MSCs form fewer colonies in monolayer than AD or TN MSCs: Of TN MSCs plated at P2, 31.4±1.6% established their own colonies (FIG. 57A), followed up by AD MSCs (29.9±2.4%) and BM MSCs (23.7±2.4%). BM MSCs formed fewer colonies than TN MSCs (p=0.0456), while AD MSCs did not differ significantly from either group (FIG. 57B). As a proxy for endpoint cellularity, DNA content of bioreactor constructs did not reveal significant differences across cell types (FIG. 57C).

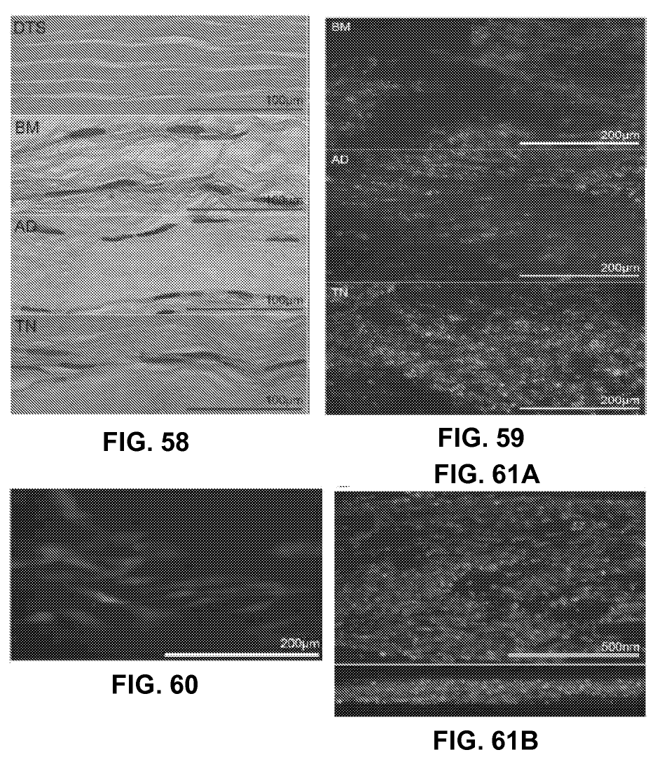

MSCs integrate into DTS during bioreactor culture: Cells exhibited elongated, tenocytic morphologies in parallel with the axis of scaffold anisotropy, with extensive cell-cell contacts (FIGS. 58-61B). Confocal microscopy revealed a dense population of live cells extending 100 μm or deeper into all scaffolds. Although all groups had similar cellularity, AD MSCs appeared to reside in the more superficial level of DTS, while BM and TN MSCs tended to integrate more deeply into the scaffold.

Gene Expression Profiles During Bioreactor-Induced Tenogenesis Differ by MSC Source:

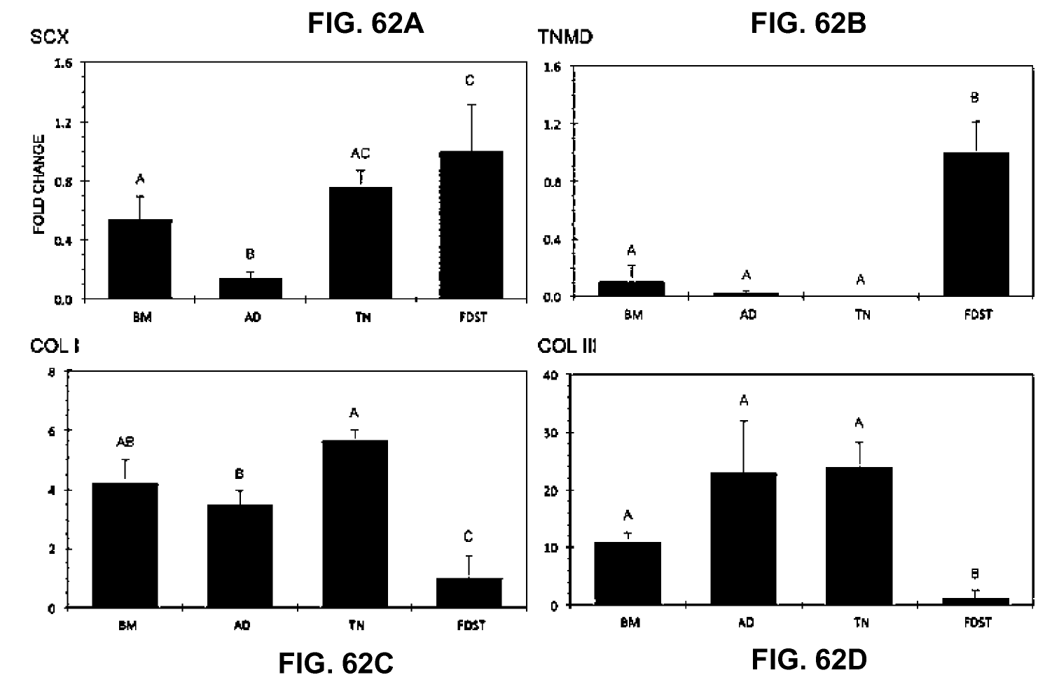

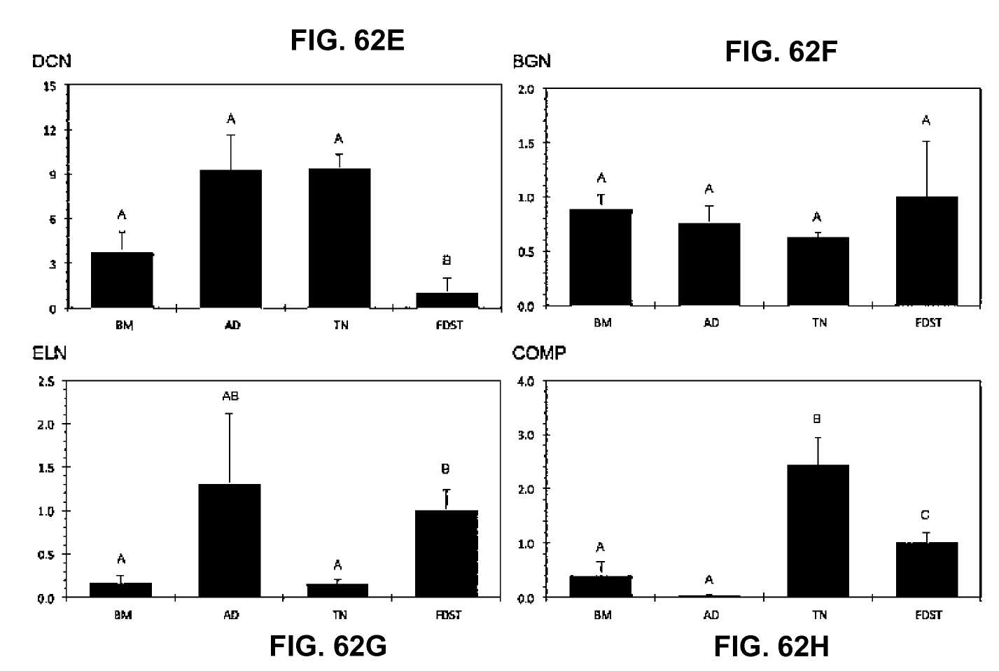

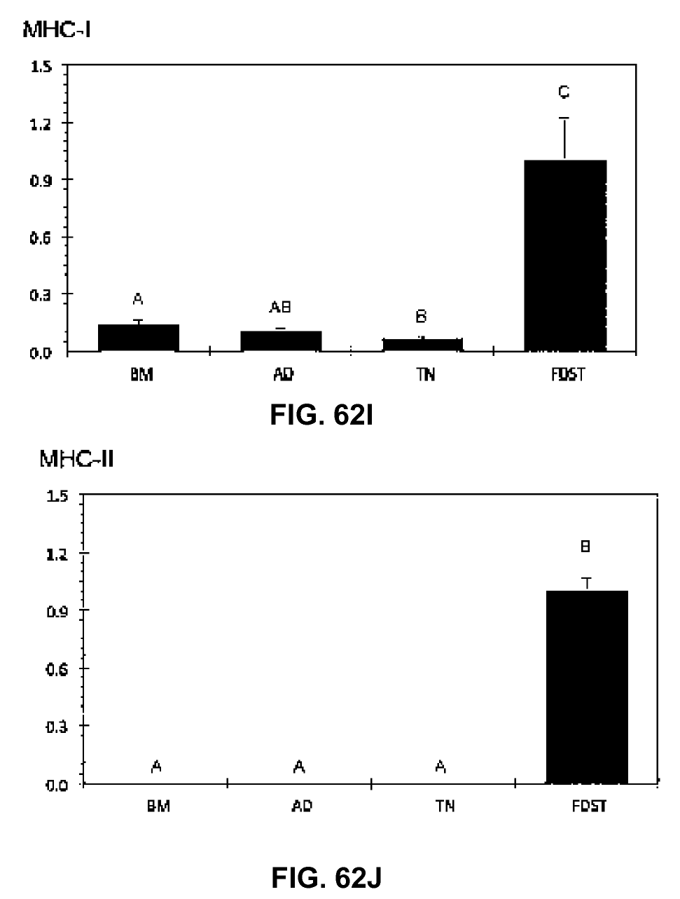

Relative gene expression data is shown in FIGS. 62A-62J. SCX was most highly expressed in the TN group, reaching 76±11% of the level of FDST, a difference that did not reach significance (p=0.1336). SCX expression in the BM and TN groups was statistically greater than in the AD group (p=0.0133 and p=0.0099, respectively). TNMD expression was at least an order of magnitude less in all groups than FDST, though no differences were detected across cell types. COL-I expression was greatest in the TN group and lowest in the AD group, with the BM group falling between. Expression of COL-I was significantly greater than FDST in all groups, but this effect was most pronounced in the TN group (p=0.0003). Similarly, COL-III expression was greater than FDST in all groups.

DCN was upregulated in all groups. While DCN expression trended lower in the BM group, this difference did not reach significance relative to the AD and TN groups (p=0.0815 and p=0.0797, respectively), and was still greater than FDST (p=0.0306). BGN was expressed at native levels in all groups. ELN expression was greatest in the AD group, and lower than FDST in the BM and TN groups (p=0.0056 and p=0.0065, respectively). COMP expression was 2.43±0.52-fold greater than FDST in the TN group (p=0.0035), and was less than FDST in the BM and AD groups (p=0.0070 and p=0.0015, respectively).

MHC-I expression was approximately an order of magnitude less in all groups versus FDST. MHC-I expression in the TN group was lower than in the BM group (p=0.0085), with the AD group falling between. MHC-II expression was not detected in any of the groups.

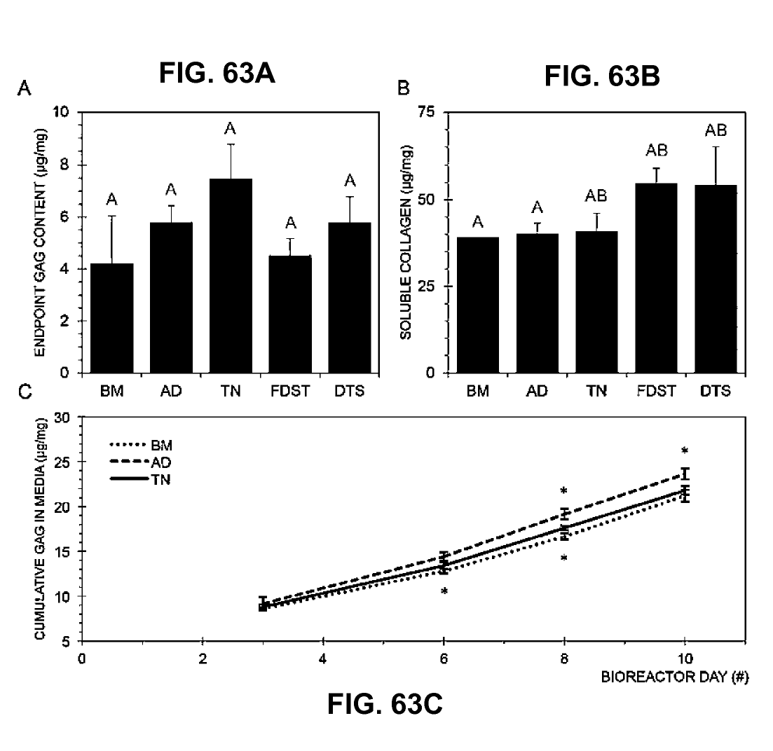

Scaffold composition was not heavily influenced by MSC cell source: Differences in ECM gene expression profiles did not contribute to detectable differences in construct GAG content, as high baseline levels of these components within DTS complicate identification of cellular contributions (FIG. 63A). Soluble collagen content was not statistically different between groups, but the BM and AD groups experienced small but significant (p=0.0311 and p=0.0246, respectively) losses in collagen content versus FDST (FIG. 63B). Small but significant differences were detected in GAG release into the cell culture media: GAG release was lower in the BM group on days 6 and 8 and higher in the AD group on days 8 and 10 (FIG. 63C). Differences in cell integration patterns may be the source of these transient changes.

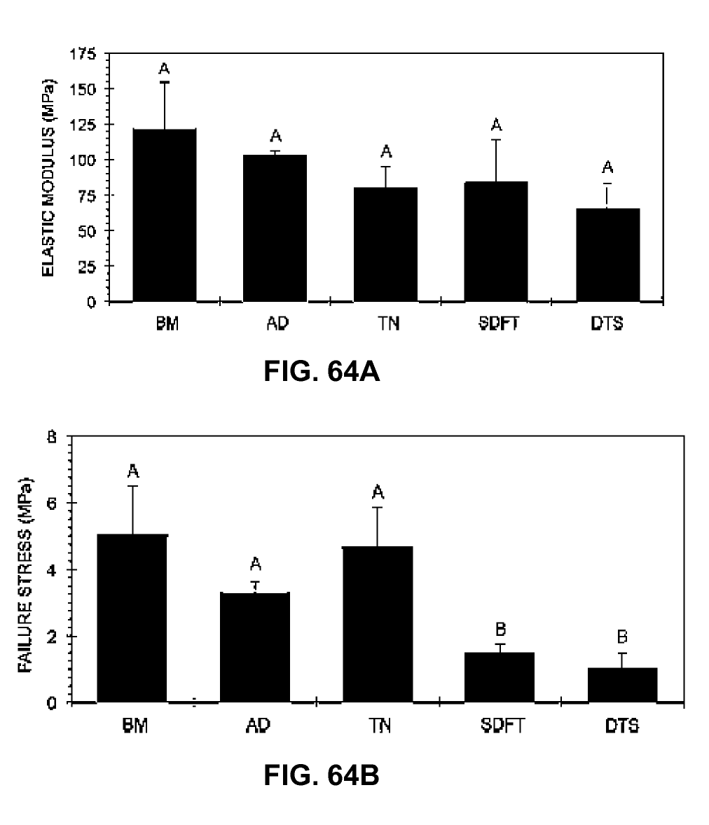

Construct mechanical properties was altered by resident cells: No differences in elastic modulus were observed during failure testing (FIG. 64A). Notably, cell-laden constructs significantly increased in tensile strength relative to controls (FIG. 64B). The BM, AD and TN groups all failed at higher stresses than FDST (p=0.0473, p=0.0177 and p=0.0440, respectively). Daily monitoring of stress/strain via bioreactor load cells did not show notable differences in construct stress over time (data not shown).

We claim:

1. A method of preparing a stromal vascular fraction (SVF) adipose stem cell population, the method comprising:
incubating an amount of adipose tissue in a digestion media containing collagenase until no adipose tissue fragments larger than about 3 mm³ remain in the digestion media to form an autologous adipose tissue digest;
centrifuging the adipose tissue digest to obtain a supernatant and a SVF pellet;
removing the supernatant;
resuspending the SVF pellet in a volume of adipose culture medium to form a SVF cell suspension; and
expanding cells in the SVF cell suspension through 6 to 8 cell divisions without passaging to form the SVF adipose stem cell population.

2. The method of claim 1, wherein the adipose tissue is harvested from a mammal.

3. The method of claim 1 or 2, wherein the adipose tissue is obtained from a human, a dog, or an equine.

4. The method of claim 1 or 2, wherein the SVF adipose stem cell population is autologous, allogeneic, syngeneic, or xenogeneic.

5. The method of claim 1 or 2, wherein the SVF adipose stem cell population is generated within 12-14 days of harvesting the adipose tissue.

6. The method of claim 1, further comprising the step of harvesting the SVF adipose stem cell population and resuspending the harvested SVF adipose stem cell population in a volume of platelet rich plasma (PRP).

7. The method of claim 6, wherein the PRP contains a ratio of platelets and leukocytes, wherein the ratio of platelets to leukocytes ranges from about 1000:0.2 to about 1000:10 (platelets:leukocytes×10³ platelets/cells per microliter).

8. The method of any one of claims 1, further comprising the step of administering at least some cells from the SVF adipose stem cell population to a subject in need thereof.

9. The method of claim 8, wherein the subject in need thereof has a tendon injury.

10. The method of any one of claim 8 or 9, wherein the subject in need thereof is a human, a dog, or a horse.

11. The method of any one of claim 8 or 9, wherein the least some cells from the SVF adipose stem cell population are administered to the subject in need thereof, intra-articularly, intravenously, or intralesionally.

12. The method of claim 1, further comprising the steps of harvesting at least some cells the SVF adipose stem cell population and seeding the harvested cells onto a natural scaffold or synthetic scaffold to form a seeded scaffold.

13. The method of claim 12, wherein the scaffold is a tendon graft.

14. The method of any one of claim 12 or 13, further comprising culturing the seeded scaffold in a soft tissue bioreactor.

15. The method of claim 14, further comprising the step of applying an unilateral axial force to the seed scaffold.

16. The method of claim 1, further comprising the step of harvesting the SVF adipose stem cell population and resuspending the harvested SVF adipose stem cell population in a volume of conditioned serum.

17. The method of claim 16, further comprising administering a composition as in any of claim 6, 7 or 16 to a subject in need thereof.

18. The method of claim 17, wherein the subject in need thereof has a tendon injury.

19. The method of any of claim 17 or 18, wherein the subject in need thereof is a human, a dog, or a horse.

20. A composition comprising:
an effective amount stem cells; and
an effective amount a plasma rich platelet composition comprising:
an effective ratio of platelets and leukocytes, wherein the effective ratio of platelets to leukocytes ranges from about 1000:0.2 to about 1000:10 (platelets:leukocytes×10³ platelets/cells per microliter).

21. The composition of claim 20, wherein the stem cells are selected from the group consisting of: bone marrow mesenchymal stem cells, cultured adipose stem cells, stromal vascular fraction adipose stem cells, and tendon precursor cells.

22. The composition of claim 20, wherein the stem cells are derived from a mammal.

23. The composition of claim 22, wherein the mammal is a human, a canine or an equine.

24. The composition of claim 20, further comprising a three dimensional scaffold, wherein the three dimensional scaffold is seeded with the stem cells.

25. The composition of claim 24, wherein the three dimensional scaffold comprises an autologous or allogeneic tendon graft.

26. The composition of claim 24, wherein the three dimensional scaffold comprises collagen.

27. The composition of claim 24, wherein the three dimensional scaffold is synthetic or natural.

28. The composition of claim 20, wherein the platelets are activated platelets.

29. The composition of claim 20, wherein the stem cells are autologous, allogeneic, xenogeneic, or syngeneic.

30. A composition comprising:
an effective amount of an stem cells; and
conditioned serum, wherein the conditioned serum comprises the serum resulting from clotting a plasma rich platelet composition comprising:
an effective ratio of platelets and leukocytes, wherein the effective ratio of platelets to leukocytes ranges from 1000:0.2 to 1000:10 (×10³ platelets/cells per microliter).

31. The composition of claim 30, wherein the stem cells are selected from the group consisting of: bone marrow mesenchymal stem cells, cultured adipose stem cells, stromal vascular fraction adipose stem cells, and tendon precursor cells.

32. The composition of claim 30, wherein the stem cells are derived from a mammal.

33. The composition of claim 30, wherein the stem cells are derived from a mammal.

34. The composition of claim 33, wherein the mammal is a canine or an equine.

35. The composition of claim 30, further comprising a three dimensional scaffold, wherein the three dimensional scaffold is seeded with the stem cells.

36. The composition of claim 35, wherein the three dimensional scaffold comprises an autologous or allogeneic tendon graft.

37. The composition of claim 35, wherein the three dimensional scaffold comprises collagen.

38. The composition of claim 15, wherein the three dimensional scaffold is a synthetic or a natural scaffold.

39. A method of treating a soft tissue injury comprising:
administering an amount of a composition as in any one of claims 20-38 to a subject in need thereof.

40. The method of claim 39, wherein the amount is an amount effective to reduce the cross sectional area of a tendon lesion.

41. The method of claim 39, wherein the amount is administered directly to the site of injury.

42. The method of claim 39, wherein administering is performed a selected number of times ranging from 1 to 10.

43. The method of claim 39, wherein the soft tendon injury is tendinopathy.

44. A method of preparing a platelet rich plasma composition, the method comprising:
centrifuging a volume of whole blood to obtain at least a plasma fraction that contains platelets and a buffy coat fraction that contains leukocytes;
removing the plasma fraction without removing any of the buffy coat fraction;
centrifuging the plasma fraction to form a platelet poor plasma (PPP) fraction and a platelet pellet;
removing a first volume of the PPP;
resuspending the platelet pellet in a second volume of PPP, where the second volume of PPP is smaller than the first volume of PPP to obtain a platelet rich plasma (PRP) composition.

45. The method of claim 44, further comprising:
adding a volume of buffy coat fraction to the platelet rich plasma composition such that the ratio of platelets to leukocytes ranges from about 1000:0.2 to about 1000:10 (platelets:leukocytes×10³ platelets/cells per microliter).

46. The method of any one of claims 44 to 45 further comprising:
mixing the PRP composition with a clotting stimulant;
incubating the PRP and the clotting stimulant from 30 minutes to about 14 hours to form a clot and a serum fraction; and
removing serum fraction to obtain a conditioned serum fraction.

47. A cell scaffold comprising:
a scaffold material seeded with stem cells, wherein the stem cells are selected from the group consisting of: bone marrow mesenchymal stem cells, cultured adipose stem cells, stromal vascular fraction adipose stem cells, and tendon precursor cells.

48. The cell scaffold of claim 47, wherein the stem cells are derived from a human, an equine, or a canine.

49. A soft tissue bioreactor comprising the cell scaffold as in any one of claims 47-48.
